(19) United States
(12) Patent Application Publication
(10) Pub. No.: US 2009/0093551 A1
(43) Pub. Date: Apr. 9, 2009

(54) REMOTELY TRIGGERED RELEASE FROM HEATABLE SURFACES

(76) Inventors: Sangeeta N. Bhatia, Lexington, MA (US); Austin M. Derfus, San Diego, CA (US); Geoffrey von Maltzahn, Boston, MA (US)

Correspondence Address: CHOATE, HALL & STEWART LLP, TWO INTERNATIONAL PLACE, BOSTON, MA, 02110, US

(21) Appl. No.: 11/952,993

(22) Filed: Dec. 7, 2007

Related U.S. Application Data
(60) Provisional application No. 60/969,389, filed on Aug. 31, 2007.
(60) Provisional application No. 60/873,897, filed on Dec. 8, 2006.

(51) Int. Cl. A61K 47/48

(52) U.S. Cl. 514/773; 514/777; 514/784

(57) ABSTRACT

The present invention provides particle conjugates for drug delivery. Such conjugates comprise one or more heatable surfaces, one or more thermally-responsive linkers, and one or more agents to be delivered. In some embodiments, conjugates and populations of conjugates can be used to treat and/or diagnose a disease, disorder, and/or condition. The present invention provides methods for producing and/or using thermally-responsive conjugates.

### RELATED APPLICATIONS

This application claims priority under 35 U.S.C. § 119(e) to U.S. Provisional Patent Applications 60/873,897, filed Dec. 8, 2006 (“the '897 application”), and 60/969,389, filed Aug. 31, 2007 (“the '389 application”). The entire contents of the '897 application and the '389 application are incorporated herein by reference in their entirety.

### GOVERNMENT SUPPORT

The United States Government has provided grant support utilized in the development of the present invention. In particular, National Institutes of Health (contract numbers N01-C0-37117, R01-CA-124427-01, U54 CA119349, U54 CA119335, and EB 006324) have supported development of this invention. The United States Government has certain rights in the invention.

### BACKGROUND OF THE INVENTION

Conventional modes of drug administration include oral, intravenous, pulmonary, and transdermal delivery. Typically, materials designed for such methods of drug delivery are engineered to have characteristic release or degradation properties given their in vivo environments. However, these agents are passive in their delivery and lack the ability to deliver payload in response to external commands (Sengupta et al., 2005, Nature, 436:568; incorporated herein by reference). Such externally-timed drug delivery is currently limited primarily to treatment via repeated administration.

The primary means of externally timed drug administration is the use of multiple injections, a treatment that remains standard practice for multi-step vaccinations, timed hormone dosing, and for treatment of diseases such as diabetes. Frequent re-administration in inconvenient for patients and caregivers and often leads to patient non-compliance.

Implantable microchips with addressable, drug-containing wells are currently being developed. These wells are individually opened for programmable or externally controlled delivery, allowing multiple drugs to be simultaneously expelled. However, this technology requires the additional burden of a permanent implant with a power supply, electronic wiring, and non-degradable scaffold.

Ultrasound can be used either to locally burst vesicle-coated bubbles that contain drug or to physically erode a hard, hydrophobic drug-containing polymer implant. Both methods have aroused questions about the safety of repeated ultrasound exposure and are limited in their ability to delivering protein or hydrophilic drugs. Additionally, the cavitation of vesicle-coated bubbles cannot be used to deliver drugs long after administration and is more suited to targeted delivery. Furthermore, because the bubble sizes become unstable below approximately 100 nm, such methods have limited ability to deliver drug beyond the endothelium.

Several other systems utilize temperature-sensitive, drug-doped hydrogels for controlled delivery. One such material can be designed such that at temperatures beyond a critical temperature, they collapse and release any soluble materials inside. Many different means of obtaining the required temperature increase have been investigated, ranging from the use of externally applied heating pads to various internal sources of heat. Often these methods lack either sufficient means of controlling the extent of release or sufficiently localizing heat so as to avoid heating body tissues. Additionally, these schemes have limited ability to delivery hydrophobic drugs.

Hydrogel approaches are limited by the fact that they have a single transition temperature. By restricting a system to one transition temperature, it is not possible to controllably deliver a variety of drug combinations by releasing different drugs at different temperatures. Additionally, approaches requiring hydrogels are not easily amenable for design as injectable, targeted delivery platforms. Furthermore, current means for thermally regulated delivery rely on conformation changes in surrounding hydrogels with micron size limitations. Additionally, because hydrogels do not physically immobilize their contents, the drugs continually diffuse out of the gel over time, preventing strict on/off modulation of release.

To date, implantable devices have been synthesized to facilitate scheduled release of multiple payloads via surface degradation (Wood et al., 2006, Proc. Natl. Acad. Sci., USA, 103:10207; incorporated herein by reference) or via programmable electronically controlled microchips. While these approaches provide local release of a bioactive payload, their dimensions preclude external activation of release to targeted regions.

Thus, there is a need in the art for methods which offer the ability to safely and precisely release a variety of drugs from a non-permanent carrier in response to external signals. There is a need in the art for improved methods for controlled drug release that decrease non-specific drug release. There is a need in the art for methods for drug delivery that enable one-time administration of multi-injection treatments to allow for convenient dosage modulation for continuous treatment of disease (e.g. diabetes).

### SUMMARY OF THE INVENTION

The present invention provides a novel means of remotely and/or controllably releasing an agent to be delivered (e.g. therapeutic, diagnostic, prophylactic, and/or nutraceutical agent). In general, heatable surfaces which heat in response to external stimuli (e.g. electromagnetic (EM) fields, light, etc.) are provided. Heatable surfaces are typically associated with one or more agents to be delivered via thermally-responsive linkers. When the resulting thermally-responsive conjugate is subjected to an external stimulus (e.g. EM field, light), heatable surfaces release a certain amount of heat. The amount of heat released may or may not be sufficient to disrupt the function of the thermally-responsive linker, resulting in release of the agent to be delivered.

In some embodiments, a heatable surface comprises any substance that can be heated. In some embodiments, a heatable surface comprises any material experiencing local or macroscopic temperature change. In some embodiments, a heatable surface comprises electromagnetically or optically responsive material. In some embodiments, a heatable surface comprises any substance that is heated in electromagnetic (EM) fields. In some embodiments, a heatable surface comprises any substance that is heated in response to light.

In some embodiments, heatable surfaces are particles (e.g. nanoparticles, microparticles, etc.). In some embodiments, a heatable surface comprises a metal nanoparticle (e.g. gold) which experiences inductive heating in an EM field. In some embodiments, the heatable surface is a magnetic nanoparticle. In general, a particle in accordance with the present invention is any entity having a greatest dimension (e.g. diameter) of less than 100 microns (μm). In some embodiments, particles have a greatest dimension of less than 10 μm, 1000 nanometers (nm), 900 nm, 800 nm, 700 nm, 600 nm, 500 nm, 400 nm, 300 nm, 200 nm, or 100 nm. Typically, particles have a greatest dimension (e.g., diameter) of 300 nm or less.

In some embodiments, heatable surfaces are nanorods, nanorings, and/or nanoshells. In some embodiments, heatable surfaces are macroscopic surfaces (e.g. sheets or blocks of metal) which can be heated in response to EM fields and/or other stimuli.

In some embodiments, heatable surfaces have detectable properties and/or are attached to detectable moieties. Such heatable surfaces allow for detection of thermally-responsive conjugates coincident with or subsequent to therapeutic administration of the conjugates. In some embodiments, detectable heatable surfaces are magnetically detectable. In some embodiments, detectable heatable surfaces are optically detectable.

The present invention provides thermally-responsive linkers which mediate the association between an agent to be delivered and a heatable surface in a temperature-sensitive manner. For example, when exposed to temperatures below a characteristic temperature or characteristic temperature range (referred to herein as the “trigger temperature”), a thermally-responsive linker can mediate the association between an agent to be delivered and a heatable surface. When the thermally-responsive linker and/or a conjugate comprising a thermally-responsive linker is exposed to the trigger temperature and/or temperatures higher than the trigger temperature, the thermally-responsive linker is no longer capable of mediating the association between the two or more entities (i.e. the thermally-responsive linker is “disrupted”), and the agent to be delivered is released from the heatable surface.

Any substance that is responsive to changes in temperature (e.g. displays different properties at different temperatures) may be a thermally-responsive linker in accordance with the present invention. In some embodiments, thermally-responsive linkers comprise at least two individual components which interact with one another in a temperature-sensitive manner. In some embodiments, thermally-responsive linkers mediate the association of a conjugate assembly in which disruption of the conjugate assembly results in release of the agent to be delivered. In some embodiments, thermally-responsive linkers comprise a single component which mediates the association of two or more moieties (e.g. heatable surfaces) in a temperature-sensitive manner. In some embodiments, thermally-responsive linkers comprise at least one individual component which has a temperature-sensitive three-dimensional conformation. In some embodiments, thermally-responsive linkers comprise nucleic acids; peptides and/or proteins; carbohydrates; and/or polymers. In certain embodiments, thermally-responsive linkers comprise complimentary Watson-Crick base pairing of nucleic acid strands (e.g. DNA, RNA, and/or PNA strands). In certain embodiments, thermally-responsive linkers comprise nucleic acids whose properties result from the three-dimensional structure of the nucleic acid (e.g. an aptamer). In certain embodiments, thermally-responsive linkers comprise interactions between complimentary peptides, lipids, polymers, and/or carbohydrates. In certain embodiments, thermally-responsive linkers comprise proteins which can undergo temperature dependent conformational changes.

In certain embodiments, a thermally-responsive linker comprises any material that swells and/or shrinks in response to temperature changes. In certain embodiments, a thermally-responsive linker comprises any material that swells and/or shrinks in response to temperature changes and also that does not break in response to temperature changes. For example, such a thermally-responsive linker may include a polymer such as pNIPAM.

Disruption of the linker typically occurs at sites where temperature triggers are present. For example, when a conjugate comprising a thermally-responsive linker is exposed to a trigger temperature, disruption of the linker leads to separation of the heatable surface and agent to be delivered. Whereas, without exposure to the trigger temperature, the agent to be delivered remains associated with the particle.

In some embodiments, disruption of the linker occurs at temperatures higher than ambient temperature. In some embodiments, disruption of the linker occurs at temperatures higher than body temperature.

The present invention encompasses the recognition that thermally-responsive linkers may be modulated such that the agent to be delivered is releases at different trigger temperatures. Such modulation enables production of thermally-responsive linkers having a specific and/or desired trigger temperature and enables multiplexing of several different drug release schemes.

In some embodiments, thermally-responsive linkers may include nucleic acid residues. In some embodiments, the trigger temperature can be modulated by varying the number of complimentary hybridizing bases on two or more nucleic acid strands. In some embodiments, the duplex region does not comprise any nucleotide mismatches. In some embodiments, the duplex region may be interrupted by 1, 2, 3, 4, 5, or more nucleotide mismatches. In some embodiments, the nucleotide mismatches may be contiguous (i.e. mismatches are adjacent to one another). In some embodiments, the nucleotide mismatches may be non-contiguous (i.e. mismatches are separated by one or more base pairs). In general, the presence of mismatches decreases the trigger temperature relative to the absence of mismatches.

In some embodiments, a thermally-responsive linker comprises a duplex region and at least one single-stranded nucleic acid overhang on either side or both sides of the duplex region. In some embodiments, the trigger temperature can be modulated by varying the nucleotide content of the nucleic acid strands. For example, increasing the amount of guanine and/or cytosine relative to the amount of adenine, thymine, and/or uracil tends to raise the trigger temperature of a thermally-responsive linker. Likewise, increasing the amount of adenine, thymine, and/or uracil relative to the amount of guanine and/or cytosine tends to lower the trigger temperature of a thermally-responsive linker. In some embodiments, the trigger temperature can be modulated by including one or more modified nucleotide residues.

In some embodiments, thermally-responsive linkers include amino acid residues. In some embodiments, protein and/or peptide linkers may comprise two or more moieties that interact with one another in a heat-sensitive manner. Protein-based interactions may be heat-sensitive if their association is at least partially-mediated by hydrogen bonding. In some embodiments, thermally-responsive linkers may include any protein-protein interaction domains that involve hydrogen bonding. In certain embodiments, thermally-responsive linkers may be based on coil geometries (e.g. α-helices, leucine zippers, collagen helices, etc.), β-sheet motifs (e.g. amphiphilic peptides), etc.

In some embodiments, protein and/or peptide linkers may comprise any heat-sensitive affinity interaction. In certain embodiments, protein and/or peptide linkers may comprise ligand-receptor interactions (e.g. TGFα-EGF receptor interactions). In some embodiments, protein and/or peptide linkers may comprise antibody-antigen interactions. In some embodiments, protein and/or peptide linkers may comprise other types of affinity interactions (e.g. any two proteins which specifically bind to one another).

In some embodiments, thermally-responsive linkers include carbohydrates.

In some embodiments, thermally-responsive linkers include polymers (e.g. synthetic polymers). In some embodiments, polymer-based embodiments encompass sol-gel hydrogels whose transition is based on temperature, including natural polymers, poly(ethylene oxide)/poly (propylene oxide) block copolymers, N-isopropylacrylamide copolymers, etc. In general, a sol-gel hydrogel refers to a class of polymer that can change from a solution to a gel under a particular set of conditions that are specific for the identity of the given polymer. In some embodiments, polymer-based thermally-responsive linkers may comprise multiphase hydrogels (see, e.g., Ehrick et al., 2005, Nat. Mater., 4:298; incorporated herein by reference).

In some embodiments, thermally-responsive linkers are hybrid linkers. In some embodiments, the term “hybrid linkers” refers to thermally-responsive linkers comprise at least two of the following: nucleic acids, proteins/peptides, carbohydrates, lipids, polymers, and small molecules.

In some embodiments, thermally-responsive linkers comprise at least two individual components which associate with one another below the trigger temperature, but do not associate with one another at and/or above the trigger temperature. Typically, one individual component is associated with the heatable surface, and another individual component is associated with the agent to be delivered. In some embodiments, the association is covalent. In some embodiments, the association is non-covalent (e.g. hydrogen bonding, charge interactions, affinity interactions, van der Waals forces, etc.).

In certain embodiments, thermally-responsive linkers comprise at least two complementary nucleic acid strands (e.g. DNA, RNA, PNA, and/or combinations thereof). In some embodiments, heat labile linkers may comprise interactions among proteins and/or peptides having coil geometries (e.g. α-helices, leucine zippers, collagen helices, etc.), β-sheet motifs (e.g. amphiphilic peptides), etc. In some embodiments, heat labile linkers may comprise a ligand-receptor interaction. In some embodiments, heat labile linkers may comprise an antibody-antigen interaction. In some embodiments, heat labile linkers may comprise an enzyme-substrate interaction. In some embodiments, heat labile linkers may comprise another type of affinity interaction (e.g. an interaction between any proteins which specifically bind to one another).

In some embodiments, thermally-responsive linkers mediate the association of a conjugate assembly for which disruption of the conjugate assembly results in release of the agent to be delivered. In some embodiments, conjugate assemblies may enable triggered enhancement of component transport or clearance. For example, a conjugate assembly may be too large for clearance from the body, but the individual conjugates within the assembly may be small enough for clearance from the body.

The present invention encompasses the recognition that thermally-responsive linkers may be modulated such that the agent to be delivered is releases at different trigger temperatures, enabling multiplexing of several different drug release schemes. For example, the nucleotide content of nucleic acid thermally-responsive linkers may be modified such that a set of linkers is generated, in which each member of the set is characterized by a different nucleotide content (e.g. nucleotide sequence) and, consequently, a different trigger temperature.

In some embodiments, thermally-responsive linkers comprise at least one individual component which has a temperature-sensitive three-dimensional conformation. In certain embodiments, thermally-responsive linkers comprise proteins and/or peptides which can undergo temperature-dependent conformational changes. In some embodiments, protein and/or peptide structures containing hydrogen bonds (e.g. α-helices, β-sheets, amphiphilic peptides, etc.) encapsulate hydrophobic agents in the interior of the structures and, upon disassociation (e.g. upon exposure to a trigger temperature), release the agents to be delivered. In some embodiments, release can occur because the protein and/or peptide structure is no longer able to contain the agent to be delivered (e.g. the agent to be delivered can “leak out” of the protein and/or peptide structure).

In some embodiments, protein and/or peptide structures may associate with agents to be delivered in a manner that is dependent on the three-dimensional structure of the protein (and/or peptide) and/or the agent to be delivered. In some embodiments, release can occur because the protein and/or peptide structure no longer associates with the agent to be delivered.

According to the present invention, thermally-responsive conjugates may be used for delivery of any agent, including, for example, therapeutic, diagnostic, prophylactic, and/or nutraceutical agents. One of ordinary skill in the art will appreciate that any agent can be delivered by the compositions and methods in accordance with the present invention. In some embodiments, agents to be delivered may include any molecule, material, substance, or construct that may be transported into a cell by conjugation to a nano- or micro-structure. Exemplary agents to be delivered in accordance with the present invention include, but are not limited to, small molecules, organometallic compounds, nucleic acids (e.g. DNA, RNA, peptide nucleic acids, etc.), proteins (including multimeric proteins, protein complexes, etc.), peptides, lipids, carbohydrates, hormones, metals, radioactive elements and compounds, hydrophobic drugs, hydrophilic drugs, vaccines, immunological agents, organic constructs, inorganic constructs, inhibitors, catalysts, nanoparticles, microparticles, etc., and/or combinations thereof. In some embodiments, the agent to be delivered may be a mixture of pharmaceutically active agents.

In some embodiments, thermally-responsive conjugates in accordance with the present invention comprise one or more targeting moieties. In general, a targeting moiety is any moiety that binds to a component associated with an organ, tissue, cell, subcellular locale, and/or extracellular matrix component. A targeting moiety may be a nucleic acid, polypeptide, glycoprotein, carbohydrate, lipid, etc. For example, a targeting moiety can be a nucleic acid targeting moiety (e.g. an aptamer) that binds to a cell type specific marker. In general, an aptamer is an oligonucleotide (e.g., DNA, RNA, or an analog or derivative thereof) that binds to a particular target, such as a polypeptide. In some embodiments, a targeting moiety may be a naturally occurring or synthetic ligand for a cell surface receptor, e.g., a growth factor, hormone, LDL, transferrin, etc. A targeting moiety can be an antibody, which term is intended to include antibody fragments, characteristic portions of antibodies, single chain antibodies, etc. Synthetic binding proteins such as affibodies, etc., can be used. Peptide targeting moieties can be identified, e.g., using procedures such as phage display. This widely used technique has been used to identify cell specific ligands for a variety of different cell types. Nanoparticle conjugates comprising targeting moieties are described in further detail in co-pending U.S. patent application entitled “DELIVERY OF NANOPARTICLES AND/OR AGENTS TO CELLS,” filed Dec. 6, 2007 (the entire contents of which are incorporated herein by reference and are attached hereto as Appendix A).

In some embodiments, targeting moieties bind to an organ, tissue, cell, extracellular matrix component, and/or intracellular compartment that is associated with a specific developmental stage or a specific disease state (i.e. a “target” or “marker”).

In some embodiments, populations of thermally-responsive conjugates are “single-component” systems. In other words, “single component” conjugates comprise heatable surfaces, thermally-responsive linkers, and/or agents to be delivered that are all identical to one another. In some embodiments, conjugate systems are “two-component” or “multi-component” conjugate systems. In other words, “two-component” or “multi-component” conjugate systems (e.g. conjugate populations, pluralities of conjugates, etc.) comprise heatable surfaces, thermally-responsive linkers, and/or agents to be delivered that are not all identical to one another.

In some embodiments, a single thermally-responsive conjugate may comprise a particle associated with multiple different thermally-responsive linkers and multiple different agents to be delivered. In some embodiments, the multiple different thermally-responsive linkers are sensitive to different temperatures. In some embodiments, such a conjugate may be used to deliver different therapeutic agents at different points in time (i.e. a dosage schedule).

The present invention provides methods of triggering disassembly of dendrimer-like conjugate assemblies connected via heat-liable linkers. Controlled disassociation of conjugate assemblies enables timed cargo release from large aggregates for the purpose of sensing, MRI, catalysis, delivery of localized high drug dosage, gene therapy, or facilitating body clearance of particles in vivo.

In some embodiments, individual conjugates within a population of conjugates interact and/or associate with one another to form assemblies of conjugates. In some embodiments, a population of conjugates comprises assemblies of individual conjugates. In some embodiments, conjugate assemblies may be characterized as having an ordered structure. In some embodiments, conjugate assemblies may be characterized as having an unordered structure.

In some embodiments, thermally-responsive conjugates may be manufactured using any available method. Methods of forming heatable surfaces (e.g. magnetic particles) are known in the art. In general, assembly of conjugates involves at least one chemical reaction. For example, attaching the agent to be delivered to the thermally-responsive linker may take place in one reaction, and attaching the heatable surface to a thermally-responsive linker may take place in a second reaction. From this point, the conjugates are formed by self-assembly, which can be performed in a controlled manner by dictating the concentrations of the individual components (e.g. heatable surfaces, thermally-responsive linkers, agents to be delivered, etc.).

In some embodiments, a heatable surface and a thermally-responsive linker are physically associated with one another. In some embodiments, a thermally-responsive linker and an agent to be delivered are physically associated with one another. In some embodiments, a heatable surface and an agent to be delivered are physically associated with one another. In some embodiments, a heatable surface and a targeting moiety are physically associated with one another. In some embodiments, a thermally-responsive linker and a targeting moiety are physically associated with one another. In some embodiments, an agent to be delivered and a targeting moiety are physically associated with one another. In certain specific embodiments, a heatable surface, thermally-responsive linker, and agent to be delivered are physically associated with one another. In certain specific embodiments, a heatable surface, thermally-responsive linker, agent to be delivered, and targeting moiety are physically associated with one another.

Physical association can be achieved in a variety of different ways. Physical association may be covalent or non-covalent. In some embodiments, non-covalent physical association may be characterized by association with the surface of, encapsulated within, surrounded by, and/or distributed throughout the polymeric matrix of a heatable surface. In some embodiments, a heatable surface, thermally-responsive linker, and/or agent to be delivered may be directly conjugated to one another or may be conjugated by means of one or more linkers.

In some embodiments, a composition in accordance with the invention is administered to a subject for therapeutic, diagnostic, and/or prophylactic purposes. In some embodiments, the amount of thermally-responsive conjugate and/or population of thermally-responsive conjugates is sufficient to treat, alleviate symptoms of, diagnose, prevent, and/or delay the onset of a disease, condition, and/or disorder. In some embodiments, the invention encompasses “therapeutic cocktails,” including, but not limited to, approaches in which multiple thermally-responsive conjugates are administered. The present invention provides thermally-responsive conjugates that enable delivery of an agent (e.g. therapeutic, diagnostic, and/or prophylactic agent) at a specific time. An agent to be delivered, as described herein, may be released from conjugates free in the bloodstream, from conjugates in tissues, from conjugates in cells, from conjugates within a hydrogel, from conjugates immobilized onto a surface, and/or from conjugates behind a membrane. Conjugates may be used in vitro as well as in vivo.

To give but a few examples, applications include intelligent drug delivery, controllable drug implants, simplified vaccinations, more potent cancer treatments, enhanced sensing capabilities, MRI, gene therapy, monitoring enzyme catalysis of endogenous and/or delivered substrates, delivery of high drug or cargo dosages to single points, reduction of non-specific drug release, localized release of growth factors to cells, intracellular cargo delivery, and/or controlled vehicle disassembly for easing clearance of particles in vivo.

Thermally responsive conjugates in accordance with the present invention and pharmaceutical compositions thereof may be administered using any amount and any route of administration effective for treatment. In some embodiments, pharmaceutical compositions in accordance with the present invention are administered by a variety of routes, including oral, intravenous, intramuscular, intra-arterial, intramedullary, intrathecal, subcutaneous, intraventricular, transdermal, interdermal, rectal, intravaginal, intraperitoneal, topical (e.g. by powders, ointments, creams, gels, lotions, and/or drops), mucosal, nasal, bucal, enteral, sublingual; by intratracheal instillation, bronchial instillation, and/or inhalation; and/or as an oral spray, nasal spray, and/or aerosol.

In some embodiments, the present invention provides for pharmaceutical compositions comprising thermally-responsive conjugates as described herein and one or more pharmaceutically acceptable excipients. Such pharmaceutical compositions may optionally comprise one or more additional therapeutically-active substances. In accordance with some embodiments, a method of administering a pharmaceutical composition comprising thermally-responsive conjugates to a subject in need thereof is provided.

The invention provides a variety of kits for conveniently and/or effectively carrying out methods in accordance with the present invention. Kits in accordance with the invention typically comprise one or more thermally-responsive conjugates. In some embodiments, kits comprise a collection of different thermally-responsive conjugates to be used for different purposes (e.g. diagnostics, treatment, and/or prophylaxis). Kits may include additional components or reagents. For example, kits may comprise one or more tools and/or pieces of equipment for exposing thermally-responsive conjugates to an EM field. In some embodiments, such a kit is used in the treatment, diagnosis, and/or prophylaxis of a subject suffering from and/or susceptible to a disease, condition, and/or disorder (e.g. cancer). In some embodiments, such a kit comprises (i) a thermally-responsive conjugate that is useful in the treatment of cancer; (ii) a syringe, needle, applicator, etc. for administration of the to a subject; and (iii) instructions for use.

### BRIEF DESCRIPTION OF THE DRAWING

FIG. 1: Schematic diagram of a thermally-responsive linker which comprises two complementary nucleic acid strands. One nucleic acid strand is associated with the heatable surface, and a second nucleic acid strand is associated with the agent to be delivered. A portion of each nucleic acid strand is complementary to the other strand. When the temperature is below a characteristic trigger temperature, the complementary portions anneal to form a duplex region. When the conjugate is subjected to a radio frequency (RF) magnetic field, the conjugate is heated to the trigger temperature or to temperatures higher than the trigger temperature. The two strands of the thermally-responsive linker denature and dissociate from one another, and the agent to be delivered is released from the heatable surface.

FIG. 2: Schematic diagram of a thermally-responsive linker which mediates the association of a conjugate assembly for which disruption of the conjugate assembly results in release of the agent to be delivered. The agent to be delivered is attached to a single-stranded nucleic acid acting as a linker between one single-stranded nucleic acid bound to one particle and a second single-stranded nucleic acid bound to a second particle. When the conjugate is placed in an EM field capable of heating the particles to and/or above the trigger temperature, the nucleic acid duplexes are disrupted, releasing the linker nucleic acid and the agent to be delivered while disassociating the particles from each other.

FIG. 3: EM field-triggered release of nanoparticle-tethered dye in pulsatile and multistage profiles. Superparamagnetic nanoparticles transduce external electromagnetic energy to heat, thereby melting oligonucleotide duplexes that act as thermally-responsive tethers to model drugs. (A) Thermally responsive conjugates comprising particles, linkers, and fluorophores were assembled by first covalently linking a 30 bp “parent” strand, and then allowing a fluorescent complement (of 12 bp, 18 bp, or 24 bp) to hybridize. (B) In vitro, nanoparticles hybridized to fluorescein-conjugated 18 bp were embedded in hydrogel plugs. Repeated EM field pulses of 5 minutes resulted in corresponding release of fluorescein. Alteration of oligonucleotide duplex length shifts response of thermally-responsive tether enabling complex release profiles. Low power EM field exposure results in release of FAM-conjugated 12 bp whereas higher power results in simultaneous melting of both 12 bp and 24 bp tethers (C).

FIG. 4: EM field-induced temperature rise varies with particle concentration and sample diameter. Experimental data (open circles) were collected by applying maximum EM field (3 kW power) to solutions of various diameters (D) containing various concentration of magnetic particles (ρ). These data were fit to a conductive heat transfer equation (inset), where k is thermal conductivity (for water: 0.64 W/m·° C.), and q is the heating rate (mW/mg). With a threshold of 5° C. temperature rise to trigger release, these results estimate a minimum of 1.2 mg particles must be delivered to a 1 cm diameter tumor.

FIG. 5: Triggered release from thermally-responsive conjugates in vivo. Conjugates were mixed with matrigel and injected subcutaneously near the posterior mammary fat pad of mice, forming tumor phantoms (A). Application of EM field to implanted phantoms with 18 bp tethers resulted in release of model drugs and penetration into surrounding tissue (B) when compared to unexposed controls (C, scale bar=100 microns). These mice were imaged with a 7T MRI scanner, and a transverse section is shown in (D) (arrow indicates tumor phantom).

### DEFINITIONS

Agent to be delivered: As used herein, the phrase “agent to be delivered” refers to any substance that can be delivered to an organ, tissue, cell, subcellular locale, and/or extracellular matrix locale. In some embodiments, the agent to be delivered is a biologically active agent, i.e., it has activity in a biological system and/or organism. For instance, a substance that, when administered to an organism, has a biological effect on that organism, is considered to be biologically active. In particular embodiments, where an agent to be delivered is a biologically active agent, a portion of that agent that shares at least one biological activity of the agent as a whole is typically referred to as a “biologically active” portion. In some embodiments, an agent to be delivered is a therapeutic agent. As used herein, the term “therapeutic agent” refers to any agent that, when administered to a subject, has a beneficial effect. The term “therapeutic agent” refers to any agent that, when administered to a subject, has a therapeutic, diagnostic, and/or prophylactic effect and/or elicits a desired biological and/or pharmacological effect. As used herein, the term “therapeutic agent” may be a therapeutic, diagnostic, prophylactic, and/or nutraceutical agent.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. In some embodiments, the moieties are attached to one another by one or more covalent bonds. In some embodiments, the moieties are attached to one another by a mechanism that involves specific (but non-covalent) binding (e.g. streptavidin/avidin interactions, antibody/antigen interactions, etc.). In some embodiments, a sufficient number of weaker interactions can provide sufficient stability for moieties to remain physically associated.

Biocompatible: As used herein, the term “biocompatible” refers to substances that are not toxic to cells. In some embodiments, a substance is considered to be “biocompatible” if its addition to cells in vivo does not induce inflammation and/or other adverse effects in vivo. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death.

Biodegradable: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

Heatable surface: As used herein, the term “heatable surface” refers to any substance capable of heating upon exposure to an external stimulus. In general, a heatable surface is a component of a thermally-responsive conjugate. One of ordinary skill in the art will appreciate that any heatable surface can be used in thermally-responsive conjugates. To give but a few examples, a heatable surface may be a magnetic, metallic, semiconductor, and/or hybrid particle (e.g. nanoparticle, microparticle, etc.). In certain embodiments, a heatable surface is a nanoparticle. In certain embodiments, a heatable surface is a microparticle. Such particles may have spherical, cubic, rod-like, ellipsoidal, plate-like, or other geometries tuned to enhance electromagnetic (EM) properties and/or to facilitate targeting and/or delivery. In certain embodiments, a heatable surface is capable of heating in response to EM fields. In some embodiments, a heatable surface is capable of heating in response to particular frequencies. In certain embodiments, a heatable surface is capable of heating in response to light. In some embodiments, a heatable surface is capable of releasing a particular amount of heat in response to EM fields and/or light.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g. between nucleic acid molecules (e.g. DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95% or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleotide sequences can, alternatively, be determined using the GAP program in the GCG software package using an NWSgapdna.CMP matrix.

In vitro: As used herein, the term “in vitro” refers to events that occur in an artificial environment, e.g., in a test tube or reaction vessel, in cell culture, etc., rather than within an organism (e.g. animal, plant, and/or microbe).

In vivo: As used herein, the term “in vivo” refers to events that occur within an organism (e.g. animal, plant, and/or microbe).

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, 4, 5, 6, 7, 8, 9, 10, or more residues. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g. polynucleotides and residues, including nucleotides and/or nucleosides) that have not been chemically modified in order to facilitate or achieve delivery.

Particle: As used herein, the term “particle” refers to any entity having a diameter of less than 100 microns (μm). Typically, particles have a longest dimension (e.g. diameter) of 1000 nm or less (e.g. a “nanoparticle”). In general, particles have dimensions small enough to allow their uptake by eukaryotic cells. In some embodiments, particles have a diameter of 300 nm or less. In some embodiments, particles have a diameter of 200 nm or less. In some embodiments, particles have a diameter of 100 nm or less. In general, particles are greater in size than the renal excretion limit, but are small enough to avoid accumulation in the liver. In some embodiments, particles are spheres, spheroids, flat, plate-shaped, cubes, cuboids, ovals, ellipses, cylinders, cones, or pyramids. In some embodiments, particles can comprise one or more heatable surfaces. In some embodiments, magnetic particles are among the particles that are used in various embodiments. “Magnetic particles” refers to magnetically responsive particles that contain one or more metals or oxides or hydroxides thereof. Metals of use in the nanoparticles include, but are not limited to, gold, silver, iron, cobalt, zinc, cadmium, nickel, gadolinium, chromium, copper, manganese, palladium, tin, and alloys and/or oxides thereof.

Protein: As used herein, the term “protein” refers to a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, etc. In some embodiments, polypeptides may comprise natural amino acids, non-natural amino acids, synthetic amino acids, and combinations thereof. The term “peptide” is used to refer to a polypeptide having a length of less than about 100 amino acids.

Self-assembly: As used herein, the term “self-assembly” refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties.

Similarity: As used herein, the term “similarity” refers to the overall relatedness between polymeric molecules, e.g. between nucleic acid molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Small molecule: In general, a “small molecule” is understood in the art to be an organic molecule that is less than about 5 kilodaltons (Kd) in size. In some embodiments, the small molecule is less than about 3 Kd, 2 Kd, or 1 Kd. In some embodiments, the small molecule is less than about 800 daltons (D), less than about 600 D, less than about 500 D, less than about 400 D, less than about 300 D, less than about 200 D, or less than about 100 D. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, and/or condition; (5) a family history of the disease, disorder, and/or condition; (6) infection by a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” means an amount of an agent to be delivered (e.g., drug, therapeutic agent, diagnostic agent, prophylactic agent, etc.) that is sufficient, when administered to a subject suffering from or susceptible to a disease, disorder, and/or condition, to treat, diagnose, prevent, and/or delay the onset of the disease, disorder, and/or condition.

Thermally-responsive conjugate: As used herein, the term “thermally-responsive conjugate” refers to a composition comprising one or more heatable surfaces, one or more thermally-responsive linkers, and one or more agents to be delivered. In general, a thermally-responsive conjugate can be used for delivering an agent (e.g. therapeutic, diagnostic, prophylactic, and/or nutraceutical agent) to an organ, tissue, cell, subcellular locale, and/or extracellular matrix locale. Each thermally-responsive conjugate has a characteristic “trigger temperature.” The thermally-responsive conjugate releases the agent to be delivered upon exposure to temperatures at or higher than the trigger temperature.

Thermally-responsive linker: As used herein, the term “thermally-responsive linker” refers to a moiety which is capable of mediating the association between two or more entities in a temperature-sensitive manner. In some embodiments, a thermally-responsive linker mediates the association between an agent to be delivered and a heatable surface in a temperature-sensitive manner. For example, when exposed to temperatures below a characteristic temperature and/or characteristic range of temperatures (referred to herein as the “trigger temperature”), a thermally-responsive linker is capable of mediating the association between an agent to be delivered and a heatable surface. When the thermally-responsive linker and/or a conjugate comprising a thermally-responsive linker is exposed to the trigger temperature and/or temperatures higher than the trigger temperature, the thermally-responsive linker is no longer capable of mediating the association between the two or more entities, and the agent to be delivered is released from the heatable surface.

Treating: As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of a therapeutically effective amount of thermally-responsive conjugates to a subject.

### DETAILED DESCRIPTION OF CERTAIN EMBODIMENTS

The present invention provides systems and methods for controlled release of pharmaceutical cargo for the purpose of remotely actuating drug delivery. One or more agents to be delivered (e.g. drugs, therapeutic agents, prophylactic agents, diagnostic agents, etc.) are associated with heatable surfaces (e.g. particles) via thermally-responsive linkers, yielding thermally-responsive conjugates. When the thermally-responsive linker is exposed to a characteristic temperature and/or characteristic temperature range (i.e. a “trigger temperature”), the linker is disrupted and the agent is released. Thermally-responsive linkers can be designed to be disrupted at different temperatures, enabling delivery of complex drug profiles, in specific orders, over variable periods of time. The method may be used for delivery of nucleic acids (e.g. DNA, RNA, peptide nucleic acids, etc.), peptides and proteins, small molecules, drugs, inhibitors, catalysts, and nano- and micro-particles using a multitude of difference heat sources. The present invention provides systems which incorporate electromagnetically excitable particles or surfaces to allow remotely actuated drug release.

### Heat-Triggered Release

The present invention provides a novel means of controllably releasing an agent to be delivered (e.g. therapeutic, diagnostic, prophylactic, and/or nutraceutical agent). In general, heatable surfaces which heat in response to external stimuli (e.g. electromagnetic (EM) fields, light, etc.) are provided. Heatable surfaces are typically associated with one or more agents to be delivered via thermally-responsive linkers. When the resulting thermally-responsive conjugate is subjected to an external stimulus (e.g. EM field, light), heatable surfaces release a certain amount of heat. The amount of heat released may or may not be sufficient to disrupt the function of the thermally-responsive linker, resulting in release of the agent to be delivered.

In some embodiments, a heatable surface comprises a porous surface layer. For example, a thermally-responsive conjugate may comprise (i) a heatable surface comprising a porous surface layer, (ii) an agent to be delivered, and (iii) a thermally-responsive linker, wherein the agent and the linker are located underneath the porous surface layer. When the agent is released from the heatable surface upon heating to trigger temperature, the agent can diffuse through the porous surface layer.

In some embodiments, a heatable surface comprises any substance that can be heated. In some embodiments, a heatable surface comprises any material experiencing local or macroscopic temperature change. In some embodiments, a heatable surface comprises electromagnetically or optically responsive material. In some embodiments, a heatable surface comprises any substance that is heated in electromagnetic (EM) fields. In some embodiments, a heatable surface comprises any substance that is heated in response to light.

In some embodiments, a heatable surface is or comprises a particle (e.g. nanoparticle, microparticle, etc.). Nanoparticles are attractive heat sources because they can obtain tens of degrees of localized, nanoscale temperature increase without affecting the macroscopic solution temperature. Particles such as superparamagnetic iron oxide show significant heating in magnetic resonance (MR) frequency fields, making them fully compatible with the existing clinical practice of MRI.

In some embodiments, heatable surfaces span radio frequencies (e.g. magnetic materials, conductive materials, etc.). In some embodiments, heatable surfaces span optical and/or infrared frequencies (e.g. plasmonic materials, such as gold, silver, copper, and materials incorporating these elements alongside other semiconductor, inorganic, or organic materials). In some embodiments, heatable surfaces comprise nanoscale and macroscale conductive materials, semiconductor materials, and/or organic materials that absorb radio frequencies or optical energy. These materials may be tuned to absorb specific frequencies of interest by altering material composition or their shape. For example, gold nanoparticles absorb at approximately 520 nm when spherical, but rod-shapes or core-shell architectures can be tuned to absorb in the near infrared region of light (about 700 nm-about 1000 nm). Higher frequencies typically correspond to higher rate of energy deposition. In some embodiments, antennas on the scale of microns or macro scale can focus EM fields or heat inductively according to Faraday's law.

In some embodiments, heatable surfaces include materials which heat via magnetic hysteresis, Neel relaxation, and/or Brownian relaxation in radio frequency ranges (i.e. 3 Hz to 3 GHz), including but not limited to iron oxides, cobalt, hybrid doped magnetic materials, etc. In some embodiments, heatable surfaces include organic materials. In certain embodiments, heatable surfaces include organic molecules such as chromophores, fluorophores, and/or nanoparticle carriers of high densities of such molecules. In certain embodiments, heatable surfaces include optical polymers. In some embodiments, heatable surfaces include carbon nanotubes. In some embodiments, heatable surfaces include semiconductor materials (e.g. quantum dots, photonic crystals, etc.). In some embodiments, heatable surfaces include metallic materials (e.g., gold, silver, copper, and/or other plasmonic materials). In some embodiments, heatable surfaces include combination materials comprising plasmonic components and conductive materials for inductive, Joule heating. These span excitations from GHz through Infrared frequencies. In some embodiments, higher frequency can correlate with a higher temperature released from the heatable surface. In some embodiments, heatable surfaces can utilize optical excitation (e.g. 200 nm-1200 nm) or radio frequency (3 Hz to 3 GHz).

In some embodiments, heatable surfaces may be tuned to absorb specific frequencies of interest by altering composition and/or shape of the heatable surface. For example, gold nanoparticles absorb at approximately 520 nm when spherical, but rod-shapes or core-shell architectures can be tuned to absorb in the near infrared region of light (approximately 700 nm-approximately 1000 nm). Higher frequencies indeed typically correspond to higher rate of energy deposition.

In some embodiments, a heatable surface comprises a nanorod for which heat release is triggered with light. Conductive nanoparticles (e.g. gold, silver, etc.) display plasmon resonances (discussed in further detail below) that are tunable by manipulating geometry (e.g. nanorods, cubes, etc.) or particle composition (e.g. nanoshells). In some embodiments, geometry may directionally relay and/or focus EM energy into a releasable bond, enabling remote-controlled release. Due to the relative deficit of near-infrared light absorbing chromophores and scattering agents, shifting this resonance into the near-infrared enables particle actuation within biological specimens. In some embodiments, tunable linkers may be interfaced with tunable nanoparticles enabling frequency-specific, and temperature-specific release of therapeutic agents. In some embodiments, plasmonic or other nanoparticles that absorb light strongly may be utilized to efficiently capture light for conversion into heat.

EM fields can be applied using any method known in the art. For example, in optical frequencies, EM fields can be applied using a light source. In some embodiments, EM fields in optical frequencies can be applied using an endoscope, a laser, a bulb, a fiber optic, and/or combinations thereof. In some embodiments, EM fields at frequencies lower than optical frequencies can be applied using a coil, handheld device, portable source, and/or combinations thereof.

In some embodiments, heatable surfaces have detectable properties and/or are attached to detectable moieties. Such heatable surfaces allow for detection of thermally-responsive conjugates coincident with or subsequent to therapeutic administration of the conjugates. In some embodiments, detectable heatable surfaces are magnetically detectable. In some embodiments, detectable heatable surfaces are optically detectable.

In some embodiments, a heatable surface comprises a metal nanoparticle (e.g. gold) which experiences inductive heating in an EM field. In some embodiments, the heatable surface is a magnetic nanoparticle. “Magnetic particles” refers to magnetically responsive particles that contain one or more metals, oxides, and/or hydroxides thereof. Such particles typically react to magnetic force resulting from a magnetic field. A magnetic field can attract or repel particles towards or away from the source of the magnetic field, respectively, optionally causing acceleration or movement in a desired direction in space. Magnetic particles may experience heating due to Brownian relaxation and reorientation of their magnetic poles.

Magnetic particles may comprise one or more ferrimagnetic, ferromagnetic, paramagnetic, and/or superparamagnetic materials. Useful particles may be made entirely or in part of one or more materials selected from the group consisting of: iron, cobalt, nickel, niobium, magnetic iron oxides, hydroxides such as maghemite (γ-Fe₂O₃), magnetite (Fe₃O₄), feroxyhyte (FeO[OH]), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the afore-mentioned oxides or hydroxides, and mixtures of any of the foregoing. See, e.g., U.S. Pat. No. 5,916,539 for suitable synthesis methods for certain of these particles. Additional materials that may be used in magnetic particles include yttrium, europium, and vanadium.

A magnetic particle may contain a magnetic material and one or more nonmagnetic materials, which may be a metal or a nonmetal (e.g. quantum dots, ceramics, polymers comprising inorganic materials, bone-derived materials, bone substitutes, viral particles, etc.). In certain embodiments, a magnetic particle is a composite particle comprising an inner core or layer containing a first material and an outer layer or shell containing a second material, wherein at least one of the materials is magnetic. Optionally both of the materials are metals. In some embodiments, the heatable surface is a nanoshell (i.e. nanoparticle coated with metal shell) which typically absorbs specific wavelengths of incident electromagnetic energy by varying particle diameter and shell thickness. In certain embodiments, a heatable surface is an iron oxide particle, e.g., the particle has a core of iron oxide. Optionally the iron oxide is monocrystalline. In certain embodiments, the particle is a superparamagnetic iron oxide particle, e.g., the particle has a core of superparamagnetic iron oxide. In certain embodiments, the heatable surface is a gold nanoshell.

In some embodiments, a heatable surface may be a magnetically detectable particle. A magnetically detectable particle is a magnetic particle that can be detected as a consequence of its magnetic properties. In some embodiments, a magnetically detectable particle can be detected within a living cell as a consequence of its magnetic properties. Use of magnetically detectable particles allows for in vivo monitoring of particle delivery, movement, migration, uptake by the liver, clearance by the kidney, and/or degradation. The present invention provides methods for imaging and/or monitoring a patient undergoing therapeutic treatment in real time. The present invention provides methods in which a clinician is able to monitor therapeutic pharmacokinetics in real time and make decisions as to the timing of drug dosing.

An optically detectable particle is one that can be detected within a living cell using optical means compatible with cell viability. Optical detection is accomplished by detecting the scattering, emission, and/or absorption of light that falls within the optical region of the spectrum, i.e., that portion of the spectrum extending from approximately 180 nm to several microns. Optionally a sample containing cells is exposed to a source of electromagnetic energy. In some embodiments, absorption of electromagnetic energy (e.g., light of a given wavelength) by the nanoparticle or a component thereof is followed by the emission of light at longer wavelengths, and the emitted light is detected. In some embodiments, scattering of light by the nanoparticles is detected. In certain embodiments, light falling within the visible portion of the electromagnetic spectrum, i.e., the portion of the spectrum that is detectable by the human eye (approximately 400 nm to approximately 700 nm) is detected. In some embodiments, light that falls within the infrared or ultraviolet region of the spectrum is detected.

The optical property can be a feature of an absorption, emission, or scattering spectrum or a change in a feature of an absorption, emission, or scattering spectrum. The optical property can be a visually detectable feature such as, for example, color, apparent size, or visibility (i.e. simply whether or not the particle is visible under particular conditions). Features of a spectrum include, for example, peak wavelength or frequency (wavelength or frequency at which maximum emission, scattering intensity, extinction, absorption, etc. occurs), peak magnitude (e.g., peak emission value, peak scattering intensity, peak absorbance value, etc.), peak width at half height, or metrics derived from any of the foregoing such as ratio of peak magnitude to peak width. Certain spectra may contain multiple peaks, of which one is typically the major peak and has significantly greater intensity than the others. Each spectral peak has associated features. Typically, for any particular spectrum, spectral features such as peak wavelength or frequency, peak magnitude, peak width at half height, etc., are determined with reference to the major peak. The features of each peak, number of peaks, separation between peaks, etc., can be considered to be features of the spectrum as a whole. The foregoing features can be measured as a function of the direction of polarization of light illuminating the particles; thus polarization dependence can be measured. Features associated with hyper-Rayleigh scattering can be measured. Fluorescence detection can include detection of fluorescence modes.

Intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments. Such particles can have a variety of different shapes including spheres, oblate spheroids, cylinders, shells, cubes, pyramids, rods (e.g., cylinders or elongated structures having a square or rectangular cross-section), tetrapods (particles having four leg-like appendages), triangles, prisms, etc. In general, the nanoparticles should have dimensions small enough to allow their uptake by eukaryotic cells. Typically the nanoparticles have a longest straight dimension (e.g., diameter) of 200 nm or less. In some embodiments, the nanoparticles have a diameter of 100 nm or less. Smaller nanoparticles, e.g., having diameters of 50 nm or less, e.g., 5-30 nm, are used in some embodiments. In some embodiments, the term “nanoparticle” encompasses atomic clusters, which have a typical diameter of 1 nm or less and generally contain from several (e.g., 3-4) up to several hundred atoms.

Fluorescence or luminescence can be detected using any approach known in the art including, but not limited to, spectrometry, fluorescence microscopy, flow cytometry, etc. Spectrofluorometers and microplate readers are typically used to measure average properties of a sample while fluorescence microscopes resolve fluorescence as a function of spatial coordinates in two or three dimensions for microscopic objects (e.g., less than about 0.1 mm diameter). Microscope-based systems are thus suitable for detecting and optionally quantitating nanoparticles inside individual cells.

Flow cytometry measures properties such as light scattering and/or fluorescence on individual cells in a flowing stream, allowing subpopulations within a sample to be identified, analyzed, and optionally quantitated (see, e.g., Mattheakis et al., 2004, Analytical Biochemistry, 327:200; incorporated herein by reference). Multiparameter flow cytometers are available. In certain embodiments, laser scanning cytometery is used (Kamentsky, 2001, Meth. Cell Biol., 63:51; incorporated herein by reference). Laser scanning cytometry can provide equivalent data to a flow cytometer but is typically applied to cells on a solid support such as a slide. It allows light scatter and fluorescence measurements and records the position of each measurement. Cells of interest may be re-located, visualized, stained, analyzed, and/or photographed. Laser scanning cytometers are available, e.g., from CompuCyte (Cambridge, Mass.).

In certain embodiments, an imaging system comprising an epifluorescence microscope equipped with a laser (e.g., a 488 nm argon laser) for excitation and appropriate emission filter(s) is used. The filters should allow discrimination between different populations of nanoparticles used in the particular assay. For example, in one embodiment, the microscope is equipped with fifteen 10 nm bandpass filters spaced to cover portion of the spectrum between 520 and 660 nm, which would allow the detection of a wide variety of different fluorescent particles. Fluorescence spectra can be obtained from populations of nanoparticles using a standard UV/visible spectrometer.

In certain embodiments, optically detectable particles are metal particles. Metals of use in the particles include, but are not limited to, gold, silver, iron, cobalt, zinc, cadmium, nickel, gadolinium, chromium, copper, manganese, palladium, tin, and alloys thereof. Oxides of any of these metals can be used.

Certain lanthanide ion-doped particles exhibit strong fluorescence and are of use in certain embodiments. A variety of different dopant molecules can be used. For example, fluorescent europium-doped yttrium vanadate (YVO₄) particles have been produced (Beaureparie et al., 2004, Nano Letters, 4:2079; incorporated herein by reference). Such particles may be synthesized in water and are readily functionalized with biomolecules.

Noble metals (e.g., gold, silver, copper, platinum, palladium, etc.) are typically used for plasmon resonant particles, which are discussed in further detail below. For example, gold, silver, or an alloy comprising gold, silver, and optionally one or more other metals can be used. Core/shell particles (e.g., having a silver core with an outer shell of gold, or vice versa) can be used. Particles containing a metal core and a nonmetallic inorganic or organic outer shell, or vice versa, can be used. In certain embodiments, the nonmetallic core or shell comprises or consists of a dielectric material such as silica. Composite particles in which a plurality of metal particles are embedded or trapped in a nonmetal (e.g., a polymer or a silica shell) may be used. Hollow metal particles (e.g., hollow nanoshells) having an interior space or cavity are used in some embodiments. In some embodiments, a nanoshell comprising two or more concentric hollow spheres is used. Such a particle optionally comprises a core, e.g., made of a dielectric material.

In certain embodiments, at least 1%, or typically at least 5% of the mass or volume of the particle or number of atoms in the particle is contributed by metal atoms. In certain embodiments, the amount of metal in the particle, or in a core or coating layer comprising a metal, ranges from approximately 5% to 100% by mass, volume, or number of atoms, or can assume any value or range between 5% and 100%.

Certain metal particles, referred to as plasmon resonant particles, exhibit the well known phenomenon of plasmon resonance. When a metal particle (usually made of a noble metal such as gold, silver, copper, platinum, etc.) is subjected to an external electric field, its conduction electrons are displaced from their equilibrium positions with respect to the nuclei, which in turn exert an attractive, restoring force. If the electric field is oscillating (as in the case of electromagnetic radiation such as light), the result is a collective oscillation of the conduction electrons in the particle, known as plasmon resonance (Kelly et al., 2003, J. Phys. Chem. B., 107:668; Schultz et al., 2000, Proc. Natl. Acad. Sci., USA, 97:996; and Schultz, 2003, Curr. Op. Biotechnol., 14:13; all of which are incorporated herein by reference). The plasmon resonance phenomenon results in extremely efficient wavelength-dependent scattering and absorption of light by the particles over particular bands of frequencies, often in the visible range. Scattering and absorption give rise to a number of distinctive optical properties that can be detected using various approaches including visually (i.e., by the naked eye or using appropriate microscopic techniques) and/or by obtaining a spectrum, such as a scattering spectrum, extinction (scattering+absorption) spectrum, or absorption spectrum from the particle(s).

Features of the spectrum of a plasmon resonant particle (e.g., peak wavelength) depend on a number of factors, including the particle's material composition, the particle's shape and size, the surrounding medium's refractive index or dielectric properties, and the presence of other particles in the vicinity. Selection of particular particle shapes, sizes, and compositions makes it possible to produce particles with a wide range of distinguishable optically detectable properties.

Single plasmon resonant particles of sufficient size can be individually detected using a variety of approaches. For example, particles larger than about 30 nm in diameter are readily detectable under an optical microscope operating in dark-field. A spectrum from these particles can be obtained, e.g., using a CCD detector or other optical detection device. Despite their small dimensions relative to the wavelength of light, metal particles can be detected optically because they scatter light very efficiently at their plasmon resonance frequency. An 80 nm particle, for example, would be millions of times brighter than a fluorescein molecule under the same illumination conditions (Schultz et al., 2000, Proc. Natl. Acad. Sci., USA, 97:996; incorporated herein by reference). Individual plasmon resonant particles can be optically detected using a variety of approaches including near-field scanning optical microscopy, differential interference microscopy with video enhancement, total internal reflection microscopy, photo-thermal interference contrast, etc. For measurements on a population of cells, a standard spectrometer, e.g., equipped for detection of UV, visible, and/or infrared light, can be used. In certain embodiments, particles are optically detected with the use of surface-enhanced Raman scattering (SERS) (Jackson et al, 2004, Proc. Natl. Acad. Sci., USA, 101:17930; incorporated herein by reference). Optical properties of metal particles and methods for synthesis of metal particles have been reviewed (Link et al., 2003, Annu. Rev. Phys. Chem., 54:331; and Masala et al., 2004, Annu. Rev. Mater. Res., 34:41; both of which are incorporated herein by reference).

In certain embodiments, particles may comprise a bulk material that is not intrinsically fluorescent, luminescent, plasmon resonant, or magnetic, but may comprise one or more fluorescent, luminescent, or magnetic moieties. For example, a particle may comprise quantum dots, fluorescent or luminescent organic molecules, or smaller particles of a magnetic material. In some embodiments, an optically detectable moiety such as a fluorescent or luminescent dye, etc., is entrapped, embedded, or encapsulated by a particle core and/or coating layer. In some embodiments, an optically detectable moiety such as a fluorescent or luminescent dye, etc., is conjugated to a particle.

Physical Properties of Heatable Surfaces

In some embodiments, heatable surfaces comprise particles that are biodegradable and biocompatible. In general, a biocompatible substance is not toxic to cells. In some embodiments, a substance is considered to be biocompatible if its addition to cells results in less than a certain threshhold of cell death. In some embodiments, a substance is considered to be biocompatible if its addition to cells does not induce adverse effects. In general, a biodegradable substance is one that undergoes breakdown under physiological conditions over the course of a therapeutically relevant time period (e.g., weeks, months, or years). In some embodiments, a biodegradable substance is a substance that can be broken down by cellular machinery. In some embodiments, a biodegradable substance is a substance that can be broken down by chemical processes.

In some embodiments, a particle which is biocompatible and/or biodegradable may be associated with an agent to be delivered that is not biocompatible, is not biodegradable, or is neither biocompatible nor biodegradable. In some embodiments, a particle which is biocompatible and/or biodegradable may be associated with a therapeutic or diagnostic agent that is also biocompatible and/or biodegradable.

In general, a particle in accordance with the present invention is any entity having a greatest dimension (e.g. diameter) of less than 100 microns (μm). In some embodiments, particles have a greatest dimension of less than 10 μm. In some embodiments, particles have a greatest dimension of less than 1000 nanometers (nm). In some embodiments, particles have a greatest dimension of less than 900 nm, 800 nm, 700 nm, 600 nm, 500 nm, 400 nm, 300 nm, 200 nm, or 100 nm. Typically, particles have a greatest dimension (e.g., diameter) of 300 nm or less. In some embodiments, particles have a greatest dimension (e.g., diameter) of 250 nm or less. In some embodiments, particles have a greatest dimension (e.g., diameter) of 200 nm or less. In some embodiments, particles have a greatest dimension (e.g., diameter) of 150 nm or less. In some embodiments, particles have a greatest dimension (e.g., diameter) of 100 nm or less. Smaller particles, e.g., having a greatest dimension of 50 nm or less are used in some embodiments. In some embodiments, particles have a greatest dimension ranging between 5 nm and 1 μm. In some embodiments, particles have a greatest dimension ranging between 25 nm and 200 nm.

In some embodiments, particles have a diameter of approximately 1000 nm. In some embodiments, particles have a diameter of approximately 750 nm. In some embodiments, particles have a diameter of approximately 500 nm. In some embodiments, particles have a diameter of approximately 450 nm. In some embodiments, particles have a diameter of approximately 400 nm. In some embodiments, particles have a diameter of approximately 350 nm. In some embodiments, particles have a diameter of approximately 300 nm. In some embodiments, particles have a diameter of approximately 275 nm. In some embodiments, particles have a diameter of approximately 250 nm. In some embodiments, particles have a diameter of approximately 225 nm. In some embodiments, particles have a diameter of approximately 200 nm. In some embodiments, particles have a diameter of approximately 175 nm. In some embodiments, particles have a diameter of approximately 150 nm. In some embodiments, particles have a diameter of approximately 125 nm. In some embodiments, particles have a diameter of approximately 100 nm. In some embodiments, particles have a diameter of approximately 75 nm. In some embodiments, particles have a diameter of approximately 50 nm. In some embodiments, particles have a diameter of approximately 25 nm.

In certain embodiments, particles are greater in size than the renal excretion limit (e.g. particles having diameters of greater than 6 nm). In specific embodiments, particles have diameters greater than 5 nm, greater than 10 nm, greater than 15 nm, greater than 20 nm, greater than 50 nm, greater than 100 nm, greater than 250 nm, greater than 500 nm, greater than 1000 nm, or larger. In certain embodiments, particles are small enough to avoid clearance of particles from the bloodstream by the liver (e.g. particles having diameters of less than 1000 nm). In specific embodiments, particles have diameters less than 1500 nm, less than 1000 nm, less than 750 nm, less than 500 nm, less than 250 nm, less than 100 nm, or smaller. In general, physiochemical features of particles, including particle size, can be selected to allow a particle to circulate longer in plasma by decreasing renal excretion and/or liver clearance. In some embodiments, particles have diameters ranging from 5 nm to 1500 nm, from 5 nm to 1000 nm, from 5 nm to 750 nm, from 5 nm to 500 nm, from 5 nm to 250 nm, or from 5 nm to 100 nm. In some embodiments, particles have diameters ranging from 10 nm to 1500 nm, from 15 nm to 1500 nm, from 20 nm to 1500 nm, from 50 nm to 1500 nm, from 100 nm to 1500 nm, from 250 nm to 1500 nm, from 500 nm to 1500 nm, or from 1000 nm to 1500 nm. In some embodiments, particles under 100 nm may be easily endocytosed in the reticuloendothelial system (RES). In some embodiments, particles under 400 nm may be characterized by enhanced accumulation in tumors. While not wishing to be bound by any theory, enhanced accumulation in tumors may be caused by the increased permeability of angiogenic tumor vasculature relative to normal vasculature. Particles can diffuse through such “leaky” vasculature, resulting in accumulation of particles in tumors.

It is often desirable to use a population of particles that is relatively uniform in terms of size, shape, and/or composition so that each particle has similar properties. For example, at least 80%, at least 90%, or at least 95% of the particles may have a diameter or greatest dimension that falls within 5%, 10%, or 20% of the average diameter or greatest dimension. In some embodiments, a population of particles may be heterogeneous with respect to size, shape, and/or composition.

Zeta potential is a measurement of surface potential of a particle. In some embodiments, particles have a zeta potential ranging between −50 mV and +50 mV. In some embodiments, particles have a zeta potential ranging between −25 mV and +25 mV. In some embodiments, particles have a zeta potential ranging between −10 mV and +10 mV. In some embodiments, particles have a zeta potential ranging between −5 mV and +5 mV. In some embodiments, particles have a zeta potential ranging between 0 mV and +50 mV. In some embodiments, particles have a zeta potential ranging between 0 mV and +25 mV. In some embodiments, particles have a zeta potential ranging between 0 mV and +10 mV. In some embodiments, particles have a zeta potential ranging between 0 mV and +5 mV. In some embodiments, particles have a zeta potential ranging between −50 mV and 0 mV. In some embodiments, particles have a zeta potential ranging between −25 mV and 0 mV. In some embodiments, particles have a zeta potential ranging between −10 mV and 0 mV. In some embodiments, particles have a zeta potential ranging between −5 mV and 0 mV. In some embodiments, particles have a substantially neutral zeta potential (i.e. approximately 0 mV).

Particles can have a variety of different shapes including spheres, oblate spheroids, cylinders, ovals, ellipses, shells, cubes, cuboids, cones, pyramids, rods (e.g., cylinders or elongated structures having a square or rectangular cross-section), dumbbells, tetrapods (particles having four leg-like appendages), triangles, prisms, etc. In some embodiments, particles can be complex aggregates of particles characterized by any of these shapes.

In some embodiments, particles are microparticles (e.g. microspheres). In general, a “microparticle” refers to any particle having a diameter of less than 1000 μm. In some embodiments, particles are nanoparticles (e.g. nanospheres). In general, a “nanoparticle” refers to any particle having a diameter of less than 1000 nm. In some embodiments, particles are picoparticles (e.g. picospheres). In general, a “picoparticle” refers to any particle having a diameter of less than 1 nm. In some embodiments, particles are liposomes. In some embodiments, particles are micelles.

Particles can be solid or hollow and can comprise one or more layers (e.g., nanoshells, nanorings, etc.). Particles may have a core/shell structure, wherein the core(s) and shell(s) can be made of different materials. Particles may comprise gradient or homogeneous alloys. Particles may be composite particles made of two or more materials, of which one, more than one, or all of the materials possesses magnetic properties, electrically detectable properties, and/or optically detectable properties.

In certain embodiments, a particle is porous, by which is meant that the particle contains holes or channels, which are typically small compared with the size of a particle. For example a particle may be a porous silica particle, e.g., a mesoporous silica nanoparticle or may have a coating of mesoporous silica (Lin et al., 2005, J. Am. Chem. Soc., 17:4570; incorporated herein by reference). Particles may have pores ranging from about 1 nm to about 50 nm in diameter, e.g., between about 1 and 20 nm in diameter. Between about 10% and 95% of the volume of a particle may consist of voids within the pores or channels.

Particles may have a coating layer. Use of a biocompatible coating layer can be advantageous, e.g., if the particles contain materials that are toxic to cells. Suitable coating materials include, but are not limited to, natural proteins such as bovine serum albumin (BSA), biocompatible hydrophilic polymers such as polyethylene glycol (PEG) or a PEG derivative, phospholipid-(PEG), silica, lipids, polymers, carbohydrates such as dextran, other nanoparticles that can be associated with nanoparticles etc. Coatings may be applied or assembled in a variety of ways such as by dipping, using a layer-by-layer technique, by self-assembly, conjugation, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties.

In some embodiments, particles may optionally comprise one or more dispersion media, surfactants, release-retarding ingredients, or other pharmaceutically acceptable excipient. In some embodiments, particles may optionally comprise one or more plasticizers or additives.

A variety of different particles are of use in accordance with the invention. In some embodiments, particles may be intrinsically magnetic particles. In some embodiments, fluorescent or luminescent nanoparticles, particles that comprise fluorescent or luminescent moieties, and plasmon resonant particles are among the particles that are used in various embodiments. In some embodiments, polymeric particles may be used in accordance with the present invention if they heat in response to external stimuli (e.g. if particles absorb radio frequency and/or optical energy).

### Thermally-Responsive Linkers

The present invention provides thermally-responsive conjugates comprising one or more heatable surfaces, thermally-responsive linkers, and agents to be delivered. In general, a thermally-responsive linker mediates the association between an agent to be delivered and a heatable surface in a temperature-sensitive manner. For example, when exposed to temperatures below a characteristic temperature and/or range of temperatures (referred to herein as the “trigger temperature”), a thermally-responsive linker can mediate the association between an agent to be delivered and a heatable surface. When the thermally-responsive linker and/or a conjugate comprising a thermally-responsive linker is exposed to the trigger temperature and/or temperatures higher than the trigger temperature, the thermally-responsive linker is no longer capable of mediating the association between the two or more entities (i.e. the thermally-responsive linker is “disrupted”), and the agent to be delivered is released from the heatable surface.

Any substance that is responsive to changes in temperature (e.g. displays different properties at different temperatures) may be a thermally-responsive linker in accordance with the present invention. In some embodiments, thermally-responsive linkers comprise at least two individual components which interact with one another in a temperature-sensitive manner. In some embodiments, thermally-responsive linkers mediate the association of a conjugate assembly in which disruption of the conjugate assembly results in release of the agent to be delivered. In some embodiments, thermally-responsive linkers comprise a single component which mediates the association of two or more moieties (e.g. heatable surfaces) in a temperature-sensitive manner. In some embodiments, thermally-responsive linkers comprise at least one individual component which has a temperature-sensitive three-dimensional conformation. In some embodiments, thermally-responsive linkers comprise nucleic acids; peptides and/or proteins; carbohydrates; and/or polymers. In certain embodiments, thermally-responsive linkers comprise complimentary Watson-Crick base pairing of nucleic acid strands (e.g. DNA, RNA, and/or PNA strands). In certain embodiments, thermally-responsive linkers comprise nucleic acids whose properties result from the three-dimensional structure of the nucleic acid (e.g. an aptamer). In certain embodiments, thermally-responsive linkers comprise interactions between complimentary peptides, lipids, polymers, and/or carbohydrates. In certain embodiments, thermally-responsive linkers comprise proteins which can undergo temperature dependent conformational changes.

In certain embodiments, a thermally-responsive linker may include a disulfide bridge (Oishi et al., 2005, J. Am. Chem. Soc., 127:1624; incorporated herein by reference). In some embodiments, a thermally-responsive linker may include a transition metal complex that falls apart when the metal is reduced. In specific embodiments, a thermally-responsive linker may include an acid-labile thioester. In some embodiments, a thermally-responsive linker includes an aminocaproic acid (also termed aminohexanoic acid) linkage.

In certain embodiments, a thermally-responsive linker comprises any material that swells and/or shrinks in response to temperature changes. In certain embodiments, a thermally-responsive linker comprises any material that swells and/or shrinks in response to temperature changes and also that does not break in response to temperature changes. For example, such a thermally-responsive linker may include a polymer such as pNIPAM.

A thermally-responsive linker typically comprises between approximately 2 to approximately 1000 atoms, between approximately 2 to approximately 750 atoms, between approximately 2 to approximately 500 atoms, between approximately 2 to approximately 250 atoms, between approximately 2 to approximately 100 atoms, or between about 6 to about 30 atoms. In some embodiments, a thermally-responsive linker suitable for the practice of the invention may be a flexible linker. In some embodiments, a thermally-responsive linker suitable for the practice of the invention may not be a flexible linker.

Disruption of the linker typically occurs at sites where temperature triggers are present. For example, when a conjugate comprising a thermally-responsive linker is exposed to a trigger temperature, disruption of the linker leads to separation of the heatable surface and agent to be delivered. Whereas, without exposure to the trigger temperature, the agent to be delivered remains associated with the particle.

In some embodiments, disruption of the linker occurs at temperatures higher than ambient temperature. In some embodiments, disruption of the linker occurs at temperatures higher than body temperature. In some embodiments, disruption of the linker occurs at a precise temperature. In some embodiments, disruption of the linker occurs at approximately 15° C., approximately 20° C., approximately 25° C., approximately 30° C., approximately 35° C., approximately 40° C., approximately 45° C., approximately 50° C., approximately 55° C., or approximately 60° C. In some embodiments, disruption of the linker occurs at approximately 23° C., approximately 24° C., approximately 25° C., approximately 26° C., approximately 27° C., approximately 28° C., approximately 29° C., approximately 30° C., approximately 31° C., approximately 32° C., approximately 33° C., approximately 34° C., approximately 35° C., approximately 36° C., approximately 37° C., approximately 38° C., approximately 39° C., approximately 40° C., approximately 41° C., approximately 42° C., approximately 43° C., approximately 44° C., approximately 45° C., or higher. In some embodiments, disruption of the linker occurs over a range of temperatures. In some embodiments, disruption of the linker occurs at temperatures ranging between 15° C. to 20° C., between 20° C. to 25° C., between 25° C. to 30° C., between 30° C. to 35° C., between 35° C. to 40° C., or between 40° C. to 45° C.

Nucleic Acid Linkers

In some embodiments, thermally-responsive linkers include nucleic acid residues and may comprise between approximately 1 to approximately 100, between approximately 1 to approximately 50, between approximately 1 to approximately 30, between approximately 2 to approximately 20, or between approximately 2 to approximately 10 nucleic acid residues joined by phosphodiester linkages. In some embodiments, thermally-responsive linkers comprise approximately 4, approximately 6, approximately 8, approximately 10, approximately 12, approximately 14, approximately 16, approximately 18, approximately 20, approximately 22, approximately 24, approximately 26, approximately 28, approximately 30, or more nucleic acid residues joined by phosphodiester linkages.

The present invention encompasses the recognition that thermally-responsive linkers may be modulated such that the agent to be delivered is releases at different trigger temperatures. Such modulation enables production of thermally-responsive linkers having a specific and/or desired trigger temperature and enables multiplexing of several different drug release schemes (described in further detail below). In some embodiments, the trigger temperature can be modulated by varying the number of complimentary hybridizing bases on the nucleic acid strands. In this manner, an external stimulus (e.g. an EM field, light, etc.) can be introduced such that, to give but one example, a thermally-responsive linker having a 12 bp duplex region is disrupted, while a thermally-responsive linker having a longer duplex region (e.g. 14, 16, 18, 20, 22, 24, or more bp duplex region) is not disrupted.

In some embodiments, the duplex region does not comprise any nucleotide mismatches. In some embodiments, the duplex region may be interrupted by 1, 2, 3, 4, 5, or more nucleotide mismatches. In some embodiments, the nucleotide mismatches may be contiguous (i.e. mismatches are adjacent to one another). In some embodiments, the nucleotide mismatches may be non-contiguous (i.e. mismatches are separated by one or more base pairs). In general, the presence of mismatches decreases the trigger temperature relative to the absence of mismatches.

In some embodiments, a thermally-responsive linker comprises a duplex region and at least one single-stranded nucleic acid overhang on either side or both sides of the duplex region. In some embodiments, the duplex region comprises approximately 4, approximately 6, approximately 8, approximately 10, approximately 12, approximately 14, approximately 16, approximately 18, approximately 20, approximately 22, approximately 24, approximately 26, approximately 28, approximately 30, or more base pairs. In some embodiments, the single-stranded overhang comprises approximately 1, approximately 2, approximately 3, approximately 4, approximately 5, approximately 6, approximately 7, approximately 8, approximately 9, approximately 10, approximately 15, approximately 20, approximately 25, approximately 30, approximately 35, approximately 40, approximately 45, approximately 50, or more nucleotides.

In some embodiments, the trigger temperature can be modulated by varying the nucleotide content of the nucleic acid strands. For example, increasing the amount of guanine and/or cytosine relative to the amount of adenine, thymine, and/or uracil tends to raise the trigger temperature of a thermally-responsive linker. Likewise, increasing the amount of adenine, thymine, and/or uracil relative to the amount of guanine and/or cytosine tends to lower the trigger temperature of a thermally-responsive linker.

In some embodiments, the trigger temperature can be modulated by including one or more modified nucleotide residues, which are described in further detail below.

For example, locked nucleic acid (LNA), a bicyclic high-affinity RNA mimic in which the sugar ring is locked in the 3′-endo conformation by the introduction of a methylene bridge group connecting the 2′-O atom with the 4′-C atom. It has been regularly demonstrated that the incorporation of LNA into an oligonucleotide probe greatly increases the affinity of that probe for its complementary target. In some embodiments, this is expressed as an increase in melting temperature (Tₘ) and/or affinity of the oligonucleotide probe against its target. For example, whereas a given full-length DNA oligonucleotide probe for an miRNA target may have a Tₘ of 60° C., an LNA-enhanced oligonucleotide probe for the same target would have a Tₘ for target of 74° C. For LNA-enhanced oligonucleotides, the Tₘ difference between a perfectly matched target and a mismatched target is substantially higher than that observed when a DNA-based oligonucleotide is used. See, for example, Roberts et al., September 2006, Nat. Meth., vol. 3 (incorporated herein by reference). Therefore, the present invention encompasses the recognition that LNA-enhanced oligonucleotides may be used for finely controlling the trigger temperature of a given nucleic acid thermally-responsive linker.

Nucleic acids in accordance with the present invention (including nucleic acid targeting moieties and/or functional RNAs to be delivered, e.g., RNAi agents, ribozymes, tRNAs, etc., described in further detail above) may be prepared according to any available technique including, but not limited to chemical synthesis, enzymatic synthesis, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in molecular biology, v. 288 (Clifton, N.J.) Totowa, N.J.: Humana Press, 2005).

Nucleic acids in accordance with the present invention may comprise naturally occurring nucleosides, modified nucleosides, naturally occurring nucleosides with hydrocarbon linkers (e.g., an alkylene) or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides of a nucleic acid targeting moiety can be replaced with a hydrocarbon linker or a polyether linker provided that the binding affinity and selectivity of the nucleic acid targeting moiety is not substantially reduced by the substitution (e.g., the dissociation constant of the nucleic acid targeting moiety for the target should not be greater than about 1×10⁻³ M).

It will be appreciated by those of ordinary skill in the art that nucleic acids in accordance with the present invention may comprise nucleotides entirely of the types found in naturally occurring nucleic acids, or may instead include one or more nucleotide analogs or have a structure that otherwise differs from that of a naturally occurring nucleic acid. U.S. Pat. Nos. 6,403,779; 6,399,754; 6,225,460; 6,127,533; 6,031,086; 6,005,087; 5,977,089; and references therein disclose a wide variety of specific nucleotide analogs and modifications that may be used. See Crooke, S. (ed.) Antisense Drug Technology: Principles, Strategies, and Applications (1^{st} ed), Marcel Dekker; ISBN: 0824705661; 1st edition (2001) and references therein. For example, 2′-modifications include halo, alkoxy and allyloxy groups. In some embodiments, the 2′-OH group is replaced by a group selected from H, OR, R, halo, SH, SR₁, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids comprising a variety of different nucleotide analogs, modified backbones, or non-naturally occurring internucleoside linkages can be utilized in accordance with the present invention. Nucleic acids in accordance with the present invention may include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display improved properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially affected. To give but one example, modifications may be located at any position of an aptamer such that the ability of the aptamer to specifically bind to the aptamer target is not substantially affected. The modified region may be at the 5′-end and/or the 3′-end of one or both strands. For example, modified aptamers in which approximately 1-5 residues at the 5′ and/or 3′ end of either of both strands are nucleotide analogs and/or have a backbone modification have been employed. The modification may be a 5′ or 3′ terminal modification. One or both nucleic acid strands may comprise at least 50% unmodified nucleotides, at least 80% unmodified nucleotides, at least 90% unmodified nucleotides, or 100% unmodified nucleotides.

Nucleic acids in accordance with the present invention may, for example, comprise a modification to a sugar, nucleoside, or internucleoside linkage such as those described in U.S. Patent Publications 2003/0175950, 2004/0192626, 2004/0092470, 2005/0020525, and 2005/0032733 (all of which are incorporated herein by reference). The present invention encompasses the use of any nucleic acid having any one or more of the modification described therein. For example, a number of terminal conjugates, e.g., lipids such as cholesterol, lithocholic acid, aluric acid, or long alkyl branched chains have been reported to improve cellular uptake. Analogs and modifications may be tested using, e.g., using any appropriate assay known in the art, for example, to select those that result in improved efficacy of a therapeutic agent, improved specific binding of an aptamer to an aptamer target, etc. In some embodiments, nucleic acids in accordance with the present invention may comprise one or more non-natural nucleoside linkages. In some embodiments, one or more internal nucleotides at the 3′-end, 5′-end, or both 3′- and 5′-ends of the aptamer are inverted to yield a linkage such as a 3′-3′ linkage or a 5′-5′ linkage.

In some embodiments, nucleic acids in accordance with the present invention are not synthetic, but are naturally-occurring entities that have been isolated from their natural environments.

Protein Linkers

In some embodiments, thermally-responsive linkers include amino acid residues and may range from about 5 to about 5000, 5 to about 1000, about 5 to about 750, about 5 to about 500, about 5 to about 250, about 5 to about 100, about 5 to about 75, about 5 to about 50, about 5 to about 40, about 5 to about 30, about 5 to about 25, about 5 to about 20, about 5 to about 15, or about 5 to about 10 amino acids in size. As used herein, the term “peptide” refers to a polypeptide having a length of less than about 100 amino acids. Peptides from panels of peptides comprising random sequences and/or sequences which have been varied consistently to provide a maximally diverse panel of peptides may be used.

Polypeptides may contain L-amino acids, D-amino acids, or both and may contain any of a variety of amino acid modifications or analogs known in the art. Useful modifications include, e.g., terminal acetylation, amidation, etc. In some embodiments, polypeptides may comprise natural amino acids, unnatural amino acids, synthetic amino acids, and combinations thereof.

In some embodiments, protein and/or peptide linkers may comprise two or more moieties that interact with one another in a heat-sensitive manner. Protein-based interactions may be heat-sensitive if their association is at least partially-mediated by hydrogen bonding. In some embodiments, thermally-responsive linkers may include any protein-protein interaction domains that involve hydrogen bonding. In certain embodiments, thermally-responsive linkers may be based on coil geometries (e.g. α-helices, leucine zippers, collagen helices, etc.), β-sheet motifs (e.g. amphiphilic peptides), etc.

In some embodiments, protein and/or peptide linkers may comprise any heat-sensitive affinity interaction. In certain embodiments, protein and/or peptide linkers may comprise ligand-receptor interactions (e.g. TGFα-EGF receptor interactions). In some embodiments, protein and/or peptide linkers may comprise antibody-antigen interactions. In some embodiments, protein and/or peptide linkers may comprise other types of affinity interactions (e.g. any two proteins which specifically bind to one another).

Carbohydrate Linkers

In some embodiments, thermally-responsive linkers include carbohydrates. Carbohydrates may be monosaccharides, disaccharides, and/or polysaccharides. In some embodiments, carbohydrate linkers may comprise between approximately 1 to approximately 100, between approximately 1 to approximately 50, between approximately 1 to approximately 30, between approximately 2 to approximately 20, or between approximately 2 to approximately 10 monosaccharides joined by glycosidic linkages.

A carbohydrate may be natural or synthetic. A carbohydrate may also be a derivatized natural carbohydrate. In certain embodiments, a carbohydrate may be a simple or complex sugar. In certain embodiments, a carbohydrate is a monosaccharide, including but not limited to glucose, fructose, galactose, and ribose. In certain embodiments, a carbohydrate is a disaccharide, including but not limited to lactose, sucrose, maltose, trehalose, and cellobiose. In certain embodiments, a carbohydrate is a polysaccharide, including but not limited to cellulose, microcrystalline cellulose, hydroxypropyl methylcellulose (HPMC), methylcellulose (MC), dextrose, dextran, glycogen, xanthan gum, gellan gum, starch, and pullulan. In certain embodiments, a carbohydrate is a sugar alcohol, including but not limited to mannitol, sorbitol, xylitol, erythritol, malitol, and lactitol.

Polymer Linkers

In some embodiments, thermally-responsive linkers include polymers (e.g. synthetic polymers). In some embodiments, polymer-based embodiments encompass sol-gel hydrogels whose transition is based on temperature, including natural polymers, poly(ethylene oxide)/poly (propylene oxide) block copolymers, N-isopropylacrylamide copolymers, etc. In some embodiments, polymer-based thermally-responsive linkers may comprise multiphase hydrogels (see, e.g., Ehrick et al., 2005, Nat. Mater., 4:298; incorporated herein by reference).

Hybrid Linkers

In some embodiments, thermally-responsive linkers are hybrid linkers. In some embodiments, the term “hybrid linkers” refers to thermally-responsive linkers comprise at least two of the following: nucleic acids, proteins/peptides, carbohydrates, lipids, polymers, and small molecules. To give but one example, in certain embodiments, thermally-responsive linkers may comprise affinity interactions based on small molecules, carbohydrates, lipids, polymers, and/or nucleic acids interacting with peptides, proteins, glycoproteins, and/or proteoglycans. To give another example, in certain embodiments, thermally-responsive linkers may comprise affinity interactions based on small molecules, carbohydrates, lipids, polymers, peptides, proteins, glycoproteins, and/or proteoglycans interacting with nucleic acids.

Mechanisms of Action of Thermally-Responsive Linkers

Multi-Component Thermally-Responsive Linkers

In some embodiments, thermally-responsive linkers comprise at least two individual components which associate with one another below the trigger temperature, but do not associate with one another at and/or above the trigger temperature. Typically, one individual component is associated with the heatable surface, and another individual component is associated with the agent to be delivered. In some embodiments, the association is covalent. In some embodiments, the association is non-covalent (e.g. hydrogen bonding, charge interactions, affinity interactions, van der Waals forces, etc.).

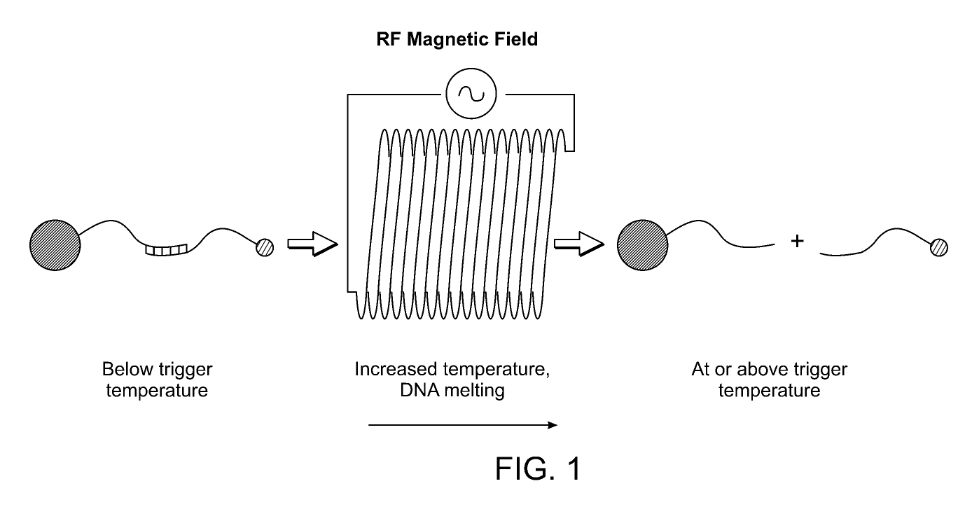

In certain embodiments, thermally-responsive linkers comprise at least two complementary nucleic acid strands (e.g. DNA, RNA, PNA, and/or combinations thereof). To give but one example, one nucleic acid strand may be associated with the heatable surface (e.g. covalently), and a second nucleic acid strand is associated with the agent to be delivered (e.g. covalently; see, for example, FIG. 1). At least a portion of each nucleic acid strand is complementary to the other strand, and the complementary portions anneal (i.e. via hydrogen bonding, to form a “duplex region”) when the temperature is below a characteristic trigger temperature. However, when exposed to the trigger temperature or to temperatures higher than the trigger temperature (e.g. when an EM field sufficiently heats the conjugate such that the trigger temperature is reached), the two strands denature and dissociate from one another (i.e. the duplex is disrupted), and the agent to be delivered is released from the heatable surface.

In some embodiments, heat labile linkers may comprise interactions among proteins and/or peptides having coil geometries (e.g. α-helices, leucine zippers, collagen helices, etc.), β-sheet motifs (e.g. amphiphilic peptides), etc. For example, one α-helix of a leucine zipper motif may be associated with the heatable surface, and the second α-helix of the leucine zipper motif may be associated with the agent to be delivered. The two α-helices associate with one another when the temperature is below a characteristic trigger temperature, forming the leucine zipper motif. However, when exposed to the trigger temperature, the two α-helices dissociate from one another, and the agent to be delivered is released from the heatable surface.

In some embodiments, heat labile linkers may comprise a ligand-receptor interaction. For example, a ligand (e.g. TGFα) may be associated with the heatable surface, and a receptor to which the ligand binds (e.g. EGF receptor) may be associated with the agent to be delivered. Alternatively, the ligand may be associated with the agent to be delivered, and the receptor may be associated with the heatable surface. The ligand and receptor associate with one another when the temperature is below a characteristic trigger temperature. However, when exposed to the trigger temperature, the ligand and receptor dissociate from one another, and the agent to be delivered is released from the heatable surface.

In some embodiments, heat labile linkers may comprise an antibody-antigen interaction. For example, an antibody may be associated with the heatable surface, and an antigen to which the antibody binds may be associated with the agent to be delivered. Alternatively, the antibody may be associated with the agent to be delivered, and the antigen may be associated with the heatable surface. The antibody and antigen associate with one another when the temperature is below a characteristic trigger temperature. However, when exposed to the trigger temperature, the antibody and antigen dissociate from one another, and the agent to be delivered is released from the heatable surface.

In some embodiments, heat labile linkers may comprise an enzyme-substrate interaction. For example, glutathione S-transferase (GST) may be associated with the heatable surface, and glutathione may be associated with the agent to be delivered. Alternatively, GST may be associated with the agent to be delivered, and glutathione may be associated with the heatable surface. GST and glutathione associate with one another when the temperature is below a characteristic trigger temperature. However, when exposed to the trigger temperature, GST and glutathione dissociate from one another, and the agent to be delivered is released from the heatable surface.

In some embodiments, heat labile linkers may comprise another type of affinity interaction (e.g. an interaction between any entities which specifically bind to one another). For example, streptavidin may be associated with the heatable surface, and biotin may be associated with the agent to be delivered. Alternatively, biotin may be associated with the agent to be delivered, and streptavidin may be associated with the heatable surface. Streptavidin and biotin associate with one another when the temperature is below a characteristic trigger temperature. However, when exposed to the trigger temperature, biotin and streptavidin dissociate from one another, and the agent to be delivered is released from the heatable surface. In some embodiments, thermally-responsive linkers may be based upon Ni-NTA interactions; peptide-metal interactions or peptide-semiconductor interactions (see, e.g., Whaley et al., 2000, Nature, 405:665; incorporated herein by reference); small molecule-target interactions; and/or adsorbed small molecule interactions.

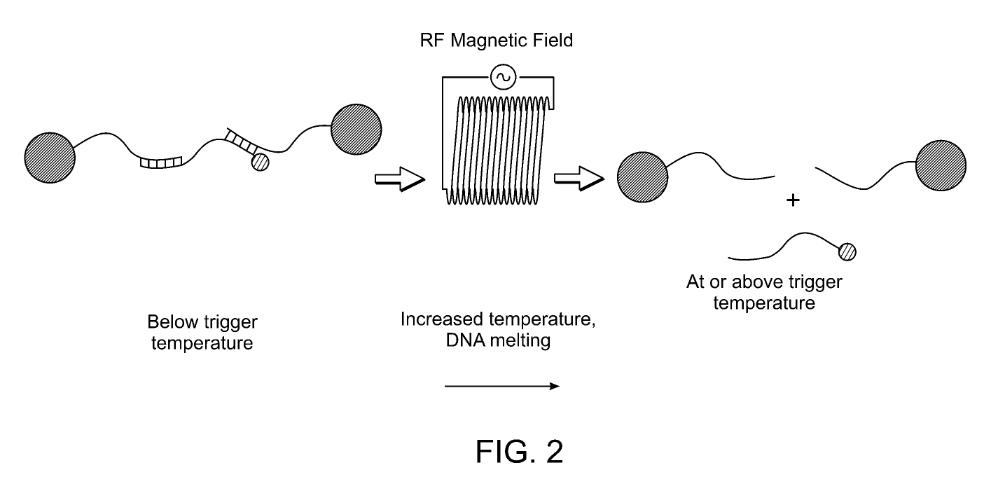

In some embodiments, thermally-responsive linkers mediate the association of a conjugate assembly for which disruption of the conjugate assembly results in release of the agent to be delivered. To give but one example, the agent to be delivered may be associated with a thermally-responsive linker which is a single-stranded nucleic acid. A heatable surface may be associated with a single-stranded nucleic acid adapter that is at least partially complementary to the thermally-responsive linker. The thermally-responsive linker, thus, is able to associate with the adapter via Watson-Crick base pairing, thereby forming a duplex region. In some embodiments, the thermally-responsive linker is able to associate with two or more adapters simultaneously, thereby joining together two or more heatable surfaces. When the conjugate is subjected to an external stimulus (e.g. placed in an EM field) which heats the particles to and/or above the trigger temperature, nucleic acid duplexes are disrupted, releasing the linker nucleic acid and the agent to be delivered while disassociating the particles from each other. FIG. 2 shows one example of such a conjugate assembly containing two particles, but one of ordinary skill in the art will recognize that the conjugate assembly may comprise many more particle linkages than one.

To give another example of a conjugate assembly, the agent to be delivered may be associated with an antigen that has multiple binding sites for an antibody (e.g. several epitopes in tandem). A heatable surface may be associated with an antibody that specifically binds to the antigen. The antigen is able to associate with several antibodies at once; thus the agent to be delivered is able to associate with two or more heatable surfaces simultaneously, thereby joining together two or more heatable surfaces. When the conjugate is subjected to an external stimulus (e.g. placed in an EM field) which heats the particles to and/or above the trigger temperature, the antibody-antigen associations are disrupted, releasing the antigen and the agent to be delivered while disassociating the particles from each other. This example described the use of antibody-antigen interactions to build such a conjugate assembly conjugate assembly, but one of ordinary skill in the art will recognize that any protein-protein interaction (e.g. affinity interaction, enzyme-substrate interaction, ligand-receptor interaction, interactions among proteins and/or peptides having coil geometries, and so forth) may be utilized to build such a conjugate assembly.

In some embodiments, conjugate assemblies may enable triggered enhancement of component transport or clearance. For example, a conjugate assembly may be too large for clearance from the body, but the individual conjugates within the assembly may be small enough for clearance from the body.

Although the specific examples provided herein relate to thermally-responsive linkers comprising nucleic acids, peptides, and/or proteins, one of ordinary skill in the art will readily recognize that conjugates in accordance with the present invention may comprise thermally-responsive linkers comprising any moieties (e.g. nucleic acids, peptides and/or proteins, carbohydrates, lipids, polymers, etc.) which associate with one another in a temperature-sensitive manner.

The present invention encompasses the recognition that thermally-responsive linkers may be modulated such that the agent to be delivered is releases at different trigger temperatures, enabling multiplexing of several different drug release schemes. For example, the nucleotide content of nucleic acid thermally-responsive linkers may be modified such that a set of linkers is generated, in which each member of the set is characterized by a different nucleotide content (e.g. nucleotide sequence) and, consequently, a different trigger temperature. Modulation of nucleic acid thermally-responsive linkers is described in further detail above, in the section entitled “Nucleic Acid Linkers.”

To give another example, the amino acid sequence of a protein thermally-responsive linker may be modified such that a set of linkers is generated in which each member of the set is characterized by a different trigger temperature. For example, for a linker that is based upon the interaction between an antibody and an antigen, the amino acid sequence of the antigen may be modified in several different ways in order to generate a set of mutated antigens. Each member of the set of antigens may have a different binding affinity for the antibody, and consequently, a different trigger temperature.

Single-Component Thermally-Responsive Linkers

In some embodiments, thermally-responsive linkers comprise at least one individual component which has a temperature-sensitive three-dimensional conformation. Such thermally-responsive linkers may include nucleic acids, peptides, proteins, carbohydrates, hybrid biopolymers (e.g. as described in the section entitled “Hybrid Linkers”), α-helical motifs, β-sheet assemblies, sol-gel polymers, etc.

In certain embodiments, thermally-responsive linkers comprise proteins and/or peptides which can undergo temperature-dependent conformational changes. In some embodiments, protein and/or peptide structures containing hydrogen bonds (e.g. α-helices, 3-sheets, amphiphilic peptides, etc.) encapsulate hydrophobic agents in the interior of the structures and, upon disassociation (e.g. upon exposure to a trigger temperature), release the agents to be delivered. In some embodiments, release can occur because the protein and/or peptide structure is no longer able to contain the agent to be delivered (e.g. the agent to be delivered can “leak out” of the protein and/or peptide structure).

In some embodiments, protein and/or peptide structures may associate with agents to be delivered in a manner that is dependent on the three-dimensional structure of the protein (and/or peptide) and/or the agent to be delivered. In some embodiments, release can occur because the protein and/or peptide structure no longer associates with the agent to be delivered.

In some embodiments, the protein and/or peptide structure is wholly denatured upon exposure to the trigger temperature. In some embodiments, the protein and/or peptide structure is only partially denatured upon exposure to the trigger temperature. In some embodiments, part of the protein and/or peptide structure is wholly denatured and part of the protein and/or peptide structure is not denatured upon exposure to the trigger temperature. In some embodiments, part of the protein and/or peptide structure is wholly denatured and part of the protein and/or peptide structure is only partially denatured upon exposure to the trigger temperature. In some embodiments, part of the protein and/or peptide structure is partially denatured and part of the protein and/or peptide structure is not denatured upon exposure to the trigger temperature.

In some embodiments, the rate of release of the agent to be delivered correlates with the extent to which the protein and/or peptide structure is denatured. In other words, more complete denaturation may result in more rapid, more effective, and/or more complete release of the agent to be delivered.

In certain embodiments, thermally-responsive linkers comprise nucleic acids whose properties result from the three-dimensional structure of the nucleic acid (e.g. an aptamer). An aptamer refers to a polynucleotide that binds to a specific target structure that is associated with a particular organ, tissue, cell, subcellular locale, and/or extracellular matrix locale. In some embodiments, agents to be delivered (e.g. small molecule drugs) can non-covalently associate with aptamers in a temperature-sensitive manner (Bagalkot et al., 200, Angew Chem. Int. Ed. Engl., 45:8149; incorporated herein by reference). In some embodiments, the agent is released from the aptamer at and/or above the trigger temperature. In some embodiments, release can occur because the aptamer no longer associates with the agent to be delivered.

In some embodiments, binding of the agent to the aptamer depends at least partly on the three-dimensional conformation of the aptamer. In some embodiments, binding of an aptamer to an agent is mediated by the interaction between the two- and/or three-dimensional structures of both the aptamer and the drug. In some embodiments, binding of an aptamer to an agent is not solely based on the primary sequence of the aptamer, but depends on the three-dimensional structure(s) of the aptamer and/or agent.

In some embodiments, an aptamer is wholly denatured upon exposure to the trigger temperature. In some embodiments, the aptamer is only partially denatured upon exposure to the trigger temperature. In some embodiments, part of the aptamer is wholly denatured and part of the aptamer is not denatured upon exposure to the trigger temperature. In some embodiments, part of the aptamer is wholly denatured and part of the aptamer is only partially denatured upon exposure to the trigger temperature. In some embodiments, part of the aptamer is partially denatured and part of the aptamer is not denatured upon exposure to the trigger temperature.

In some embodiments, the rate of release of the agent to be delivered correlates with the extent to which the aptamer is denatured. In other words, more complete denaturation may result in more rapid, more effective, and/or more complete release of the agent to be delivered.

To give but one example, some agents (e.g. doxorubicin) are known to be capable of intercalating between the bases of nucleic acid molecules. In some embodiments, an agent to be delivered may intercalate between the bases of a nucleic acid thermally-responsive linker in a temperature-sensitive manner.

Although the embodiments described above relate to proteins, peptides, and/or nucleic acid single-component thermally-responsive linkers, one of ordinary skill in the art will readily recognize that the same principles apply for single-component thermally-responsive linkers comprising carbohydrates, lipids, polymers, and/or any substance having a temperature-sensitive three-dimensional conformation.

### Agent to be Delivered

According to the present invention, thermally-responsive conjugates may be used for delivery of any agent, including, for example, therapeutic, diagnostic, prophylactic, and/or nutraceutical agents. One of ordinary skill in the art will appreciate that any agent can be delivered by the compositions and methods in accordance with the present invention. In some embodiments, agents to be delivered may include any molecule, material, substance, or construct that may be transported into a cell by conjugation to a nano- or micro-structure. Exemplary agents to be delivered in accordance with the present invention include, but are not limited to, small molecules, organometallic compounds, nucleic acids (e.g. DNA, RNA, peptide nucleic acids, etc.), proteins (including multimeric proteins, protein complexes, etc.), peptides, lipids, carbohydrates, hormones, metals, radioactive elements and compounds, hydrophobic drugs, hydrophilic drugs, vaccines, immunological agents, organic constructs, inorganic constructs, inhibitors, catalysts, nanoparticles, microparticles, etc., and/or combinations thereof.

One of ordinary skill in the art will appreciate that an agent to be delivered should retain at least part of its therapeutic effectiveness (e.g. biological and/or physiological activity) at or above the trigger temperature of the conjugate with which the agent is associated.

In some embodiments, each particle of a thermally-responsive conjugate comprises one or more agents to be delivered. In some embodiments, each particle of a thermally-responsive conjugate comprises exactly one agent to be delivered. In some embodiments, some of the particles of a population of thermally-responsive conjugates comprise one or more agents to be delivered. In some embodiments, some of the particles of a population of thermally-responsive conjugates do not comprise any agents to be delivered.

In some embodiments, conjugates comprise less than 50% by weight, less than 40% by weight, less than 30% by weight, less than 20% by weight, less than 15% by weight, less than 10% by weight, less than 5% by weight, less than 1% by weight, less than 0.5% by weight, less than 0.1% by weight, or less than 0.05% by weight of the agent to be delivered.

In some embodiments, the agent to be delivered may be a mixture of pharmaceutically active agents. For example, a local anesthetic may be delivered in combination with an anti-inflammatory agent such as a steroid. To give but another example, an antibiotic may be combined with an inhibitor of the enzyme commonly produced by bacteria to inactivate the antibiotic (e.g., penicillin and clavulanic acid).

In some embodiments, the agent to be delivered may be useful for treating growth deficiencies. For example, the agent to be delivered may be a growth hormone (e.g. human growth hormone). In some embodiments, the agent to be delivered may be useful for treating diabetes. In some embodiments, the agent to be delivered may be insulin.

In certain embodiments, the drug is an anti-atherosclerotic agent (e.g., beta-blockers, cholesterol lowering agents, etc.). In some embodiments, the drug is a cholesterol lowering agent (e.g., lovastatin, pravastatin, simvastatin, fluvastatin, atorvastatin, niacin, etc.). In some embodiments, the drug is an anti-inflammatory agent (e.g., prednisone; dexamethasone, fluorometholone; prednisolone; methylprednisolone; clobetasol; halobetasol; hydrocortisone; triamcinolone; betamethasone; fluocinolone; fluocinonide; loteprednol; medrysone; rimexolone; celecoxib; folic acid; diclofenac; diflunisal; fenoprofen; flurbiprofen; indomethacin; ketoprofen; meclofenamate; meclofamate; piroxicam; sulindac; salsalate; nabumetone; oxaprozin; tolmetin; hydroxychloroquine sulfate; rofecoxib; etanercept; infliximab; leflunomide; naproxen; oxaprozin; piroxicam; salicylates; valdecoxib; sulfasalazine; methylprednisolone; ibuprofen; budesonide, meloxicam; methylprednisolone acetate; gold sodium thiomalate; aspirin; azathioprine; triamcinolone acetonide; propoxyphene napsylate/apap; folate; nabumetone; diclofenac; ketorolac; piroxicam; etodolac; diclofenac sodium; diclofenac potassium; oxaprozin; methotrexate; minocycline; tacrolimus (FK-506); sirolimus (rapamycin) and rapamycin analogs; phenylbutazone; diclofenac sodium/misoprostol; acetaminophen; indomethacin; glucosamine sulfate/chondroitin; cyclosporin, etc.). In some embodiments, the drug is an anti-platelet agent (e.g., aspirin, clopidogrel, ticlopidine, dipyridamole, glycoprotein IIb/IIIa receptor blocker [e.g., abciximab, eptifibatide, tirofiban], cilostazol, etc.). In some embodiments, the drug is an anti-coagulant (e.g., warfarin, acenocoumarol, phenprocoumon, phenindione, heparin, low molecular weight heparin, fondaparinux, etc.). In some embodiments, the drug is an anti-proliferative agent (e.g., alkylating agents, antimetabolites, plant alkaloids, vinca alkaloids, taxanes, podophyllotoxin, topoisomerase inhibitors, hormonal therapy, antitumor antibiotics, etc.). In some embodiments, the drug is a cytotoxic agent. In certain embodiments, the drug is an immunosuppressant (e.g., glucocorticoids, cytostatics [e.g., alkylating agents, methotrexate, azathioprine, mercaptopurine], antibodies, cyclosporin, tacrolimus, sirolimus, interferons, opiods, TNF binding proteins, mycophenolate, etc.). In certain embodiments, the agent is a drug approved by the United States Food and Drug Administration (U.S.F.D.A.) for human or veterinary use.

In some embodiments, the agent to be delivered may be a mixture of anti-cancer agents. In some embodiments, thermally-responsive conjugates are administered in combination with one or more of the anti-cancer agents described herein. Combination therapy is described in further detail below, in the section entitled, “Administration.” To give but one example, in some embodiments, conjugates comprising an agent to be delivered may be administered in combination with an alkylating agent. To provide another example, compositions comprising an anti-cancer agent to be delivered are administered in combination with hormonal therapy. The growth of some types of tumors can be inhibited by providing or blocking certain hormones. For example, steroids (e.g. dexamethasone) can inhibit tumor growth or associated edema and may cause regression of lymph node malignancies. In some cases, prostate cancer is often sensitive to finasteride, an agent that blocks the peripheral conversion of testosterone to dihydrotestosterone. Breast cancer cells often highly express the estrogen and/or progesterone receptor. Inhibiting the production (e.g. with aromatase inhibitors) or function (e.g. with tamoxifen) of these hormones can often be used in breast cancer treatments. In some embodiments, gonadotropin-releasing hormone agonists (GnRH), such as goserelin possess a paradoxic negative feedback effect followed by inhibition of the release of follicle stimulating hormone (FSH) and leuteinizing hormone (LH), when given continuously.

Small Molecule Agents

In some embodiments, the agent to be delivered is a small molecule and/or organic compound with pharmaceutical activity. In some embodiments, the agent is a clinically-used drug. In some embodiments, the drug is an anti-cancer agent, antibiotic, anti-viral agent, anti-HIV agent, anti-parasite agent, anti-protozoal agent, anesthetic, anticoagulant, enzyme inhibitor, enzyme activator, steroidal agent, steroidal or non-steroidal anti-inflammatory agent, antihistamine, immunosuppressant agent, anti-neoplastic agent, antigen, vaccine, antibody, decongestant, sedative, opioid, analgesic, anti-pyretic, birth control agent, hormone, prostaglandin, progestational agent, anti-glaucoma agent, ophthalmic agent, anti-cholinergic, anti-depressant, anti-psychotic, neurotoxin, hypnotic, tranquilizer, anti-convulsant, muscle relaxant, anti-Parkinson agent, anti-spasmodic, muscle contractant, channel blocker, miotic agent, anti-secretory agent, anti-thrombotic agent, anticoagulant, anti-cholinergic, β-adrenergic blocking agent, diuretic, cardiovascular active agent, vasoactive agent, vasodilating agent, anti-hypertensive agent, angiogenic agent, modulators of cell-extracellular matrix interactions (e.g. cell growth inhibitors and anti-adhesion molecules), inhibitors of DNA, RNA, or protein synthesis, etc.

In certain embodiments, the therapeutic agent to be delivered is an anti-cancer agent (i.e. cytotoxic agents). Most anti-cancer agents can be divided in to the following categories: alkylating agents, antimetabolites, natural products, and hormones and antagonists.

Anti-cancer agents typically affect cell division and/or DNA synthesis. However, some chemotherapeutic agents do not directly interfere with DNA. To give but one example, tyrosine kinase inhibitors (imatinib mesylate/Gleevec®) directly target a molecular abnormality in certain types of cancer (chronic myelogenous leukemia, gastrointestinal stromal tumors, etc.).

Alkylating agents are so named because of their ability to add alkyl groups to many electronegative groups under conditions present in cells. Alkylating agents typically function by chemically modifying cellular DNA. Exemplary alkylating agents include nitrogen mustards (e.g. mechlorethamine, cyclophosphamide, ifosfamide, melphalan (l-sarcolysin), chlorambucil), ethylenimines and methylmelamines (e.g. altretamine (hexamethylmelamine; HMM), thiotepa (triethylene thiophosphoramide), triethylenemelamine (TEM)), alkyl sulfonates (e.g. busulfan), nitrosureas (e.g. carmustine (BCNU), lomustine (CCMU), semustine (methyl-CCNU), streptozocin (streptozotocin)), and triazenes (e.g. dacarbazine (DTIC; dimethyltriazenoimidazolecarboxamide)).

Antimetabolites act by mimicking small molecule metabolites (e.g. folic acid, pyrimidines, and purines) in order to be incorporated into newly synthesized cellular DNA. Such agents also affect RNA synthesis. An exemplary folic acid analog is methotrexate (amethopterin). Exemplary pyrimidine analogs include fluorouracil (5-fluorouracil; 5-FU), floxuridine (fluorodeoxyuridine; FUdR), and cytarabine (cytosine arabinoside). Exemplary purine analogs include mercaptopurine (6-mercaptopurine; 6-MP), azathioprine, thioguanine (6-thioguanine; TG), fludarabine phosphate, pentostatin (2′-deoxycoformycin), cladribine (2-chlorodeoxyadenosine; 2-CdA), and erythrohydroxynonyladenine (EHNA).

Natural small molecule products which can be used as anti-cancer agents include plant alkaloids and antibiotics. Plant alkaloids and terpenoids (e.g. vinca alkaloids, podophyllotoxin, taxanes, etc.) typically block cell division by preventing microtubule function. Vinca alkaloids (e.g. vincristine, vinblastine (VLB), vinorelbine, vindesine, etc.) bind to tubulin and inhibit assembly of tubulin into microtubules. Vinca alkaloids are derived from the Madagascar periwinkle, Catharanthus roseus (formerly known as Vinca rosea). Podophyllotoxin is a plant-derived compound used to produce two other cytostatic therapeutic agents, etoposide and teniposide, which prevent cells from entering the GI and S phases of the cell cycle. Podophyllotoxin is primarily obtained from the American Mayapple (Podophyllum peltatum) and a Himalayan Mayapple (Podophyllum hexandrum). Taxanes (e.g. paclitaxel, docetaxel, etc.) are derived from the Yew Tree. Taxanes enhance stability of microtubules, preventing the separation of chromosomes during anaphase.

Antibiotics which can be used as anti-cancer agents include dactinomycin (actinomycin D), daunorubicin (daunomycin; rubidomycin), doxorubicin, idarubicin, bleomycin, plicamycin (mithramycin), and mitomycin (mytomycin C).

Other small molecules which can be used as anti-cancer agents include platinum coordination complexes (e.g. cisplatin (cis-DDP), carboplatin), anthracenedione (e.g. mitoxantrone), substituted urea (e.g. hydroxyurea), methylhydrazine derivatives (e.g. procarbazine (N-methylhydrazine, M1H), and adrenocortical suppressants (e.g. mitotane (o,p′-DDD), aminoglutethimide).

Hormones which can be used as anti-cancer agents include adrenocorticosteroids (e.g. prednisone), aminoglutethimide, progestins (e.g. hydroxyprogesterone caproate, medroxyprogesterone acetate, megestrol acetate), estrogens (e.g. diethylstilbestrol, ethinyl estradiol), antiestrogen (e.g. tamoxifen), androgens (e.g. testosterone propionate, fluoxymesterone), antiandrogens (e.g. flutamide), and gonadotropin-releasing hormone analog (e.g. leuprolide).

Topoisomerase inhibitors act by inhibiting the function of topoisomerases, which are enzymes that maintain the topology of DNA. Inhibition of type I or type II topoisomerases interferes with both transcription and replication of DNA by upsetting proper DNA supercoiling. Some exemplary type I topoisomerase inhibitors include camptothecins (e.g. irinotecan, topotecan, etc.). Some exemplary type II topoisomerase inhibitors include amsacrine, etoposide, etoposide phosphate, teniposide, etc., which are semisynthetic derivatives of epipodophyllotoxins, discussed herein.

In certain embodiments, a small molecule agent can be any drug. In some embodiments, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

A more complete listing of classes and specific drugs suitable for use in the present invention may be found in Pharmaceutical Drugs: Syntheses, Patents, Applications by Axel Kleemann and Jurgen Engel, Thieme Medical Publishing, 1999 and the Merck Index: An Encyclopedia of Chemicals, Drugs and Biologicals, Ed. by Budavari et al., CRC Press, 1996, both of which are incorporated herein by reference.

Nucleic Acid Agents

In certain embodiments, thermally-responsive conjugates are used to deliver one or more nucleic acids (e.g. RNA, DNA, functional RNAs, functional DNAs, peptide nucleic acids, etc.) to a specific location such as an organ, tissue, cell, subcellular locale, and/or extracellular matrix locale.

Functional RNA

In general, a “functional RNA” is an RNA that does not code for a protein but instead belongs to a class of RNA molecules whose members characteristically possess one or more different functions or activities within a cell. It will be appreciated that the relative activities of functional RNA molecules having different sequences may differ and may depend at least in part on the particular cell type in which the RNA is present. Thus the term “functional RNA” is used herein to refer to a class of RNA molecule and is not intended to imply that all members of the class will in fact display the activity characteristic of that class under any particular set of conditions. In some embodiments, functional RNAs include RNAi-inducing entities (e.g. short interfering RNAs (siRNAs), short hairpin RNAs (shRNAs), and microRNAs (miRNAs), antagomirs, etc.), ribozymes, tRNAs, rRNAs, RNAs useful for triple helix formation, etc.

RNAi is an evolutionarily conserved process in which presence of an at least partly double-stranded RNA molecule in a eukaryotic cell leads to sequence-specific inhibition of gene expression. RNAi was originally described as a phenomenon in which the introduction of long dsRNA (typically hundreds of nucleotides) into a cell results in degradation of mRNA containing a region complementary to one strand of the dsRNA (U.S. Pat. No. 6,506,559; and Fire et al., 1998, Nature, 391:806; both of which are incorporated herein by reference). Subsequent studies in Drosophila showed that long dsRNAs are processed by an intracellular RNase III-like enzyme called Dicer into smaller dsRNAs primarily comprised of two approximately 21 nucleotide (nt) strands that form a 19 base pair duplex with 2 nt 3′ overhangs at each end and 5′-phosphate and 3′-hydroxyl groups (see, e.g., PCT Publication WO 01/75164; U.S. Patent Publications 2002/0086356 and 2003/0108923; Zamore et al., 2000, Cell, 101:25; and Elbashir et al., 2001, Genes Dev., 15:188; all of which are incorporated herein by reference).

Short dsRNAs having structures such as this, referred to as siRNAs, silence expression of genes that include a region that is substantially complementary to one of the two strands. This strand is referred to as the “antisense” or “guide” strand, with the other strand often being referred to as the “sense” strand. The siRNA is incorporated into a ribonucleoprotein complex termed the RNA-induced silencing complex (RISC) that contains member(s) of the Argonaute protein family. Following association of the siRNA with RISC, a helicase activity unwinds the duplex, allowing an alternative duplex to form the guide strand and a target mRNA containing a portion substantially complementary to the guide strand. An endonuclease activity associated with the Argonaute protein(s) present in RISC is responsible for “slicing” the target mRNA, which is then further degraded by cellular machinery.

Considerable progress towards the practical application of RNAi was achieved with the discovery that exogenous introduction of siRNAs into mammalian cells can effectively reduce the expression of target genes in a sequence-specific manner via the mechanism described above. A typical siRNA structure includes a 19 nucleotide double-stranded portion, comprising a guide strand and an antisense strand. Each strand has a 2 nt 3′ overhang. Typically the guide strand of the siRNA is perfectly complementary to its target gene and mRNA transcript over at least 17-19 contiguous nucleotides, and typically the two strands of the siRNA are perfectly complementary to each other over the duplex portion. However, as will be appreciated by one of ordinary skill in the art, perfect complementarity is not required. Instead, one or more mismatches in the duplex formed by the guide strand and the target mRNA is often tolerated, particularly at certain positions, without reducing the silencing activity below useful levels. For example, there may be 1, 2, 3, or even more mismatches between the target mRNA and the guide strand (disregarding the overhangs). Thus, as used herein, two nucleic acid portions such as a guide strand (disregarding overhangs) and a portion of a target mRNA that are “substantially complementary” may be perfectly complementary (i.e., they hybridize to one another to form a duplex in which each nucleotide is a member of a complementary base pair) or they may have a lesser degree of complementarity sufficient for hybridization to occur. One of ordinary skill in the art will appreciate that the two strands of the siRNA duplex need not be perfectly complementary. Typically at least 80%, preferably at least 90%, or more of the nucleotides in the guide strand of an effective siRNA are complementary to the target mRNA over at least about 19 contiguous nucleotides. The effect of mismatches on silencing efficacy and the locations at which mismatches may most readily be tolerated are areas of active study (see, e.g., Reynolds et al., 2004, Nat. Biotechnol., 22:326; incorporated herein by reference).

It will be appreciated that molecules having the appropriate structure and degree of complementarity to a target gene will exhibit a range of different silencing efficiencies. A variety of additional design criteria have been developed to assist in the selection of effective siRNA sequences. Numerous software programs that can be used to choose siRNA sequences that are predicted to be particularly effective to silence a target gene of choice are available (see, e.g., Yuan et al., 2004, Nucl. Acids. Res., 32:W130; and Santoyo et al., 2005, Bioinformatics, 21:1376; both of which are incorporated herein by reference).

As will be appreciated by one of ordinary skill in the art, RNAi may be effectively mediated by RNA molecules having a variety of structures that differ in one or more respects from that described above. For example, the length of the duplex can be varied (e.g., from about 17-29 nucleotides); the overhangs need not be present and, if present, their length and the identity of the nucleotides in the overhangs can vary (though most commonly symmetric dTdT overhangs are employed in synthetic siRNAs).

Additional structures, referred to as short hairpin RNAs (shRNAs), can mediate RNA interference. An shRNA is a single RNA strand that contains two complementary regions that hybridize to one another to form a double-stranded “stem,” with the two complementary regions being connected by a single-stranded loop. shRNAs are processed intracellularly by Dicer to form an siRNA structure containing a guide strand and an antisense strand. While shRNAs can be delivered exogenously to cells, more typically intracellular synthesis of shRNA is achieved by introducing a plasmid or vector containing a promoter operably linked to a template for transcription of the shRNA into the cell, e.g., to create a stable cell line or transgenic organism.

While sequence-specific cleavage of target mRNA is currently the most widely used means of achieving gene silencing by exogenous delivery of short RNAi entities to cells, additional mechanisms of sequence-specific silencing mediated by short RNA entities are known. For example, post-transcriptional gene silencing mediated by small RNA entities can occur by mechanisms involving translational repression. Certain endogenously expressed RNA molecules form hairpin structures containing an imperfect duplex portion in which the duplex is interrupted by one or more mismatches and/or bulges. These hairpin structures are processed intracellularly to yield single-stranded RNA species referred to as known as microRNAs (miRNAs), which mediate translational repression of a target transcript to which they hybridize with less than perfect complementarity. siRNA-like molecules designed to mimic the structure of miRNA precursors have been shown to result in translational repression of target genes when administered to mammalian cells.

Thus the exact mechanism by which a short RNAi entity inhibits gene expression appears to depend, at least in part, on the structure of the duplex portion of the RNAi entity and/or the structure of the hybrid formed by one strand of the RNAi entity and a target transcript. RNAi mechanisms and the structure of various RNA molecules known to mediate RNAi, e.g., siRNA, shRNA, miRNA and their precursors, have been extensively reviewed (see, e.g., Dykxhhorn et al., 2003, Nat. Rev. Mol. Cell. Biol., 4:457; Hannon et al., 2004, Nature, 431:3761; and Meister et al., 2004, Nature, 431:343; all of which are incorporated herein by reference). It is to be expected that future developments will reveal additional mechanisms by which RNAi may be achieved and will reveal additional effective short RNAi entities. Any currently known or subsequently discovered short RNAi entities are within the scope of the present invention.

A short RNAi entity that is delivered according to the methods in accordance with the invention and/or is present in a composition in accordance with the invention may be designed to silence any eukaryotic gene. The gene can be a mammalian gene, e.g., a human gene. The gene can be a wild type gene, a mutant gene, an allele of a polymorphic gene, etc. The gene can be disease-associated, e.g., a gene whose over-expression, under-expression, or mutation is associated with or contributes to development or progression of a disease. For example, the gene can be oncogene. The gene can encode a receptor or putative receptor for an infectious agent such as a virus (see, e.g., Dykxhhorn et al., 2003, Nat. Rev. Mol. Cell. Biol., 4:457 for specific examples; incorporated herein by reference).

In some embodiments, shRNAs may be used as molecular sensors. For example, shRNAs may serve as molecular beacons. In some embodiments, molecular beacons comprise nucleic acids that comprise fluorophore-quencher pairs (e.g. so that fluorescence is quenched prior to binding of a target mRNA). In certain embodiments, fluorescence is quenched when the shRNA is on the particle and bent, but dequenched when it is released and bound to its target. In certain embodiments, fluorescence is dequenched when the shRNA is on the particle and bent, but quenched when it is released and bound to its target. In such embodiments, by externally monitoring fluorescence, both the release and the intracellular binding to a target RNA of an shRNA agent may be separately monitored.

In some embodiments, tRNAs are functional RNA molecules whose delivery to eukaryotic cells can be monitored using the compositions and methods in accordance with the invention. The structure and role of tRNAs in protein synthesis is well known (Soll and Rajbhandary, (eds.) tRNA: Structure, Biosynthesis, and Function, ASM Press, 1995). The cloverleaf shape of tRNAs includes several double-stranded “stems” that arise as a result of formation of intramolecular base pairs between complementary regions of the single tRNA strand. There is considerable interest in the synthesis of polypeptides that incorporate unnatural amino acids such as amino acid analogs or labeled amino acids at particular positions within the polypeptide chain (see, e.g., Kohrer and RajBhandary, “Proteins carrying one or more unnatural amino acids,” Chapter 33, In Ibba et al., (eds.), Aminoacyl-tRNA Synthetases, Landes Bioscience, 2004). One approach to synthesizing such polypeptides is to deliver a suppressor tRNA that is aminoacylated with an unnatural amino acid to a cell that expresses an mRNA that encodes the desired polypeptide but includes a nonsense codon at one or more positions. The nonsense codon is recognized by the suppressor tRNA, resulting in incorporation of the unnatural amino acid into a polypeptide encoded by the mRNA (Kohrer et al., 2001, Proc. Natl. Acad. Sci., USA, 98:14310; and Kohrer et al., 2004, Nucleic Acids Res., 32:6200; both of which are incorporated herein by reference). However, as in the case of siRNA delivery, existing methods of delivering tRNAs to cells result in variable levels of delivery, complicating efforts to analyze such proteins and their effects on cells.

The invention contemplates the delivery of tRNAs, e.g., suppressor tRNAs, and thermally-responsive conjugates to eukaryotic cells in order to achieve the synthesis of proteins that incorporate an unnatural amino acid with which the tRNA is aminoacylated. The analysis of proteins that incorporate one or more unnatural amino acids has a wide variety of applications. For example, incorporation of amino acids modified with detectable (e.g., fluorescent) moieties can allow the study of protein trafficking, secretion, etc., with minimal disturbance to the native protein structure. Alternatively or additionally, incorporation of reactive moieties (e.g., photoactivatable and/or cross-linkable groups) can be used to identify protein interaction partners and/or to define three-dimensional structural motifs. Incorporation of phosphorylated amino acids such as phosphotyrosine, phosphothreonine, or phosphoserine, or analogs thereof, into proteins can be used to study cell signaling pathways and requirements.

In some embodiments, the functional RNA is a ribozyme. A ribozyme is designed to catalytically cleave target mRNA transcripts may be used to prevent translation of a target mRNA and/or expression of a target (see, e.g., PCT publication WO 90/11364; and Sarver et al., 1990, Science 247:1222; both of which are incorporated herein by reference).

In some embodiments, endogenous target gene expression may be reduced by targeting deoxyribonucleotide sequences complementary to the regulatory region of the target gene (i.e., the target gene's promoter and/or enhancers) to form triple helical structures that prevent transcription of the target gene (see generally, Helene, 1991, Anticancer Drug Des. 6:569; Helene et al., 1992, Ann, N.Y. Acad. Sci. 660:27; and Maher, 1992, Bioassays 14:807; all of which are incorporated herein by reference).

RNAs such as RNAi-inducing entities, tRNAs, ribozymes, etc., for delivery to eukaryotic cells may be prepared according to any available technique including, but not limited to chemical synthesis, enzymatic synthesis, enzymatic or chemical cleavage of a longer precursor, etc. Methods of synthesizing RNA molecules are known in the art (see, e.g., Gait, M. J. (ed.) Oligonucleotide synthesis: a practical approach, Oxford (Oxfordshire), Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005). Short RNAi entities such as siRNAs are commercially available from a number of different suppliers. Pre-tested siRNAs targeted to a wide variety of different genes are available, e.g., from Ambion (Austin, Tex.), Dharmacon (Lafayette, Colo.), Sigma-Aldrich (St. Louis, Mo.).

When siRNAs are synthesized in vitro the two strands are typically allowed to hybridize before contacting them with cells. It will be appreciated that the resulting siRNA composition need not consist entirely of double-stranded (hybridized) molecules. For example, an RNAi entity commonly includes a small proportion of single-stranded RNA. Generally, at least approximately 50%, at least approximately 90%, at least approximately 95%, or even at least approximately 99%-100% of the RNAs in an siRNA composition are double-stranded when contacted with cells. However, a composition containing a lower proportion of dsRNA may be used, provided that it contains sufficient dsRNA to be effective.

Vectors

In some embodiments, a nucleic acid to be delivered is a vector. As used herein, the term “vector” refers to a nucleic acid molecule (typically, but not necessarily, a DNA molecule) which can transport another nucleic acid to which it has been linked. A vector can achieve extra-chromosomal replication and/or expression of nucleic acids to which they are linked in a host cell. In some embodiments, a vector can achieve integration into the genome of the host cell.

In some embodiments, vectors are used to direct protein and/or RNA expression. In some embodiments, the protein and/or RNA to be expressed is not normally expressed by the cell. In some embodiments, the protein and/or RNA to be expressed is normally expressed by the cell, but at lower levels than it is expressed when the vector has not been delivered to the cell.

In some embodiments, a vector directs expression of any of the proteins described herein. In some embodiments, a vector directs expression of a protein with anti-cancer activity. In some embodiments, a vector directs expression of any of the functional RNAs described herein, such as RNAi-inducing entities, ribozymes, etc. In some embodiments, a vector directs expression of a functional RNA with anti-cancer activity.

Protein Agents

In some embodiments, the agent to be delivered may be a protein or peptide, as defined herein. In certain embodiments, peptides range from about 5 to about 5000, 5 to about 1000, about 5 to about 750, about 5 to about 500, about 5 to about 250, about 5 to about 100, about 5 to about 75, about 5 to about 50, about 5 to about 40, about 5 to about 30, about 5 to about 25, about 5 to about 20, about 5 to about 15, or about 5 to about 10 amino acids in size. Peptides from panels of peptides comprising random sequences and/or sequences which have been varied consistently to provide a maximally diverse panel of peptides may be used.

Polypeptides may contain L-amino acids, D-amino acids, or both and may contain any of a variety of amino acid modifications or analogs known in the art. Useful modifications include, e.g., terminal acetylation, amidation, etc. In some embodiments, polypeptides may comprise natural amino acids, unnatural amino acids, synthetic amino acids, and combinations thereof, as described herein.

In some embodiments, the agent to be delivered may be a peptide, hormone, erythropoietin, insulin, cytokine, antigen for vaccination, etc. In some embodiments, the agent to be delivered may be an antibody and/or characteristic portion thereof. In some embodiments, antibodies may include, but are not limited to, polyclonal, monoclonal, chimeric (i.e. “humanized”), single chain (recombinant) antibodies. In some embodiments, antibodies may have reduced effector functions and/or bispecific molecules. In some embodiments, antibodies may include Fab fragments and/or fragments produced by a Fab expression library, as described in further detail above.

In some embodiments, the agent to be delivered may be an anti-cancer agent.

Exemplary protein anti-cancer agents are enzymes (e.g. L-asparaginase) and biological response modifiers, such as interferons (e.g. interferon-α), interleukins (e.g. interleukin 2; IL-2), granulocyte colony-stimulating factor (G-CSF), and granulocyte/macrophage colony-stimulating factor (GM-CSF). In some embodiments, a protein anti-cancer agent is an antibody or characteristic portion thereof which is cytotoxic to tumor cells.

Carbohydrate Agents

In some embodiments, the agent to be delivered is a carbohydrate, such as a carbohydrate that is associated with a protein (e.g. glycoprotein, proteogycan, etc.). A carbohydrate may be natural or synthetic. A carbohydrate may also be a derivatized natural carbohydrate. In certain embodiments, a carbohydrate may be a simple or complex sugar. In certain embodiments, a carbohydrate is a monosaccharide, including but not limited to glucose, fructose, galactose, and ribose. In certain embodiments, a carbohydrate is a disaccharide, including but not limited to lactose, sucrose, maltose, trehalose, and cellobiose. In certain embodiments, a carbohydrate is a polysaccharide, including but not limited to cellulose, microcrystalline cellulose, hydroxypropyl methylcellulose (HPMC), methylcellulose (MC), dextrose, dextran, glycogen, xanthan gum, gellan gum, starch, and pullulan. In certain embodiments, a carbohydrate is a sugar alcohol, including but not limited to mannitol, sorbitol, xylitol, erythritol, malitol, and lactitol.

Lipid Agents

In some embodiments, the agent to be delivered is a lipid, such as a lipid that is associated with a protein (e.g. lipoprotein). Exemplary lipids that may be used in accordance with the present invention include, but are not limited to, oils, fatty acids, saturated fatty acid, unsaturated fatty acids, essential fatty acids, cis fatty acids, trans fatty acids, glycerides, monoglycerides, diglycerides, triglycerides, hormones, steroids (e.g., cholesterol, bile acids), vitamins (e.g. vitamin E), phospholipids, sphingolipids, and lipoproteins.

In some embodiments, the lipid may comprise one or more fatty acid groups or salts thereof. In some embodiments, the fatty acid group may comprise digestible, long chain (e.g., C₈-C₅₀), substituted or unsubstituted hydrocarbons. In some embodiments, the fatty acid group may be a C₁₀-C₂₀ fatty acid or salt thereof. In some embodiments, the fatty acid group may be a C₁₅-C₂₀ fatty acid or salt thereof. In some embodiments, the fatty acid group may be a C₁₅-C₂₅ fatty acid or salt thereof. In some embodiments, the fatty acid group may be unsaturated. In some embodiments, the fatty acid group may be monounsaturated. In some embodiments, the fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, the fatty acid group may be one or more of butyric, caproic, caprylic, capric, lauric, myristic, palmitic, stearic, arachidic, behenic, or lignoceric acid. In some embodiments, the fatty acid group may be one or more of palmitoleic, oleic, vaccenic, linoleic, alpha-linolenic, gamma-linoleic, arachidonic, gadoleic, arachidonic, eicosapentaenoic, docosahexaenoic, or erucic acid.

Diagnostic Agents

In some embodiments, the agent to be delivered is a diagnostic agent. In some embodiments, diagnostic agents include gases; commercially available imaging agents used in positron emissions tomography (PET), computer assisted tomography (CAT), single photon emission computerized tomography, x-ray, fluoroscopy, and magnetic resonance imaging (MRI); anti-emetics; and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

In some embodiments, thermally-responsive conjugates may comprise a diagnostic agent used in magnetic resonance imaging (MRI), such as iron oxide particles or gadolinium complexes. Gadolinium complexes that have been approved for clinical use include gadolinium chelates with DTPA, DTPA-BMA, DOTA and HP-DO3A (reviewed in Aime et al., 1998, Chemical Society Reviews, 27:19; incorporated herein by reference).

In some embodiments, thermally-responsive conjugates may comprise radionuclides as therapeutic and/or diagnostic agents. Among the radionuclides used, gamma-emitters, positron-emitters, and X-ray emitters are suitable for diagnostic and/or therapy, while beta emitters and alpha-emitters may also be used for therapy. Suitable radionuclides for forming thermally-responsive conjugates in accordance with the invention include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷ Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁷Cu, ⁷⁵Br, ⁷⁷Br, ⁹⁹ mTc, ¹⁴C, ¹³N, ¹⁵O, ³²P, ³³P, and ¹⁸F.

In some embodiments, a diagnostic agent may be a fluorescent, luminescent, or magnetic moiety. In some embodiments, a detectable moiety such as a fluorescent or luminescent dye, etc., is entrapped, embedded, or encapsulated by a particle core and/or coating layer.

Fluorescent and luminescent moieties include a variety of different organic or inorganic small molecules commonly referred to as “dyes,” “labels,” or “indicators.” Examples include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g., U.S. Patent Application Publication 2004/0067503; Valeur, B., “Molecular Fluorescence: Principles and Applications,” John Wiley and Sons, 2002; Handbook of Fluorescent Probes and Research Products, Molecular Probes, gth edition, 2002; and The Handbook—A Guide to Fluorescent Probes and Labeling Technologies, Invitrogen, 10^{th} edition, available at the Invitrogen web site; both of which are incorporated herein by reference).

Prophylactic Agents

In some embodiments, the agent to be delivered is a prophylactic agent. In some embodiments, prophylactic agents include vaccines. Vaccines may comprise isolated proteins or peptides, inactivated organisms and viruses, dead organisms and virus, genetically altered organisms or viruses, and cell extracts. Prophylactic agents may be combined with interleukins, interferon, cytokines, and adjuvants such as cholera toxin, alum, Freund's adjuvant, etc. Prophylactic agents may include antigens of such bacterial organisms as Streptococccus pnuemoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

Nutraceutical Agents

In some embodiments, the therapeutic agent to be delivered is a nutraceutical agent. In some embodiments, the nutraceutical agent provides basic nutritional value, provides health or medical benefits, and/or is a dietary supplement. In some embodiments, the nutraceutical agent is a vitamin (e.g. vitamins A, B, C, D, E, K, etc.), mineral (e.g. iron, magnesium, potassium, calcium, etc.), or essential amino acid (e.g. lysine, glutamine, leucine, etc.).

In some embodiments, nutraceutical agents may include plant or animal extracts, such as fatty acids and/or omega-3 fatty acids (e.g. DHA or ARA), fruit and vegetable extracts, lutein, phosphatidylserine, lipoid acid, melatonin, glucosamine, chondroitin, aloe vera, guggul, green tea, lycopene, whole foods, food additives, herbs, phytonutrients, antioxidants, flavonoid constituents of fruits, evening primrose oil, flaxseeds, fish and marine animal oils (e.g. cod liver oil), and probiotics.

Exemplary nutraceutical agents and dietary supplements are disclosed, for example, in Roberts et al., (Nutriceuticals: The Complete Encyclopedia of Supplements, Herbs, Vitamins, and Healing Foods, American Nutriceutical Association, 2001). Nutraceutical agents and dietary supplements are also disclosed in Physicians' Desk Reference for Nutritional Supplements, 1st Ed. (2001) and The Physicians' Desk Reference for Herbal Medicines, 1st Ed. (2001).

Those skilled in the art will recognize that this is an exemplary, not comprehensive, list of agents that can be delivered using the thermally-responsive conjugates in accordance with the present invention. Any agent may be associated with particles for remotely controlled delivery in accordance with the present invention.

### Targeting Moieties

In some embodiments, thermally-responsive conjugates in accordance with the present invention comprise one or more targeting moieties. In general, a targeting moiety is any moiety that binds to a component associated with an organ, tissue, cell, subcellular locale, and/or extracellular matrix component. In some embodiments, such a component is referred to as a “target” or a “marker,” and these are discussed in further detail below.

A targeting moiety may be a nucleic acid, polypeptide, glycoprotein, carbohydrate, lipid, etc. For example, a targeting moiety can be a nucleic acid targeting moiety (e.g. an aptamer) that binds to a cell type specific marker. In general, an aptamer is an oligonucleotide (e.g., DNA, RNA, or an analog or derivative thereof) that binds to a particular target, such as a polypeptide. In some embodiments, a targeting moiety may be a naturally occurring or synthetic ligand for a cell surface receptor, e.g., a growth factor, hormone, LDL, transferrin, etc. A targeting moiety can be an antibody, which term is intended to include antibody fragments, characteristic portions of antibodies, single chain antibodies, etc. Synthetic binding proteins such as affibodies, etc., can be used. Peptide targeting moieties can be identified, e.g., using procedures such as phage display. This widely used technique has been used to identify cell specific ligands for a variety of different cell types.

In some embodiments, targeting moieties bind to an organ, tissue, cell, extracellular matrix component, and/or intracellular compartment that is associated with a specific developmental stage or a specific disease state (i.e. a “target” or “marker”). In some embodiments, a target is an antigen on the surface of a cell, such as a cell surface receptor, an integrin, a transmembrane protein, an ion channel, and/or a membrane transport protein. In some embodiments, a target is an intracellular protein. In some embodiments, a target is a soluble protein, such as immunoglobulin. In some embodiments, a target is more prevalent, accessible, and/or abundant in a diseased locale (e.g. organ, tissue, cell, subcellular locale, and/or extracellular matrix component) than in a healthy locale. To give but one example, in some embodiments, a target is preferentially expressed in tumor tissues versus normal tissues. In some embodiments, a target is more prevalent, accessible, and/or abundant in locales (e.g. organs, tissues, cells, subcellular locales, and/or extracellular matrix components) associated with a particular developmental state than in locales associated with a different developmental state. In some embodiments, targeting moieties facilitate the passive entry into target sites by extending circulation time of conjugates, reducing non-specific clearance of conjugates, and/or geometrically enhancing the accumulation of conjugates in target sites.

In some embodiments, a targeting moiety in accordance with the present invention may be a nucleic acid. As used herein, a “nucleic acid targeting moiety” refers to a nucleic acid that binds selectively to a target. In some embodiments, a nucleic acid targeting moiety is a nucleic acid aptamer. An aptamer is typically a polynucleotide that binds to a specific target structure that is associated with a particular organ, tissue, cell, subcellular locale, and/or extracellular matrix component. In general, the targeting function of the aptamer is based on the three-dimensional structure of the aptamer and/or target.

In some embodiments, a targeting moiety in accordance with the present invention may be a small molecule. In certain embodiments, small molecules are less than about 2000 g/mol in size. In some embodiments, small molecules are less than about 1500 g/mol or less than about 1000 g/mol. In some embodiments, small molecules are less than about 800 g/mol or less than about 500 g/mol. One of ordinary skill in the art will appreciate that any small molecule that specifically binds to a desired target can be used in accordance with the present invention.

In some embodiments, a targeting moiety in accordance with the present invention may be a protein or peptide. In certain embodiments, peptides range from about 5 to 100, 10 to 75, 15 to 50, or 20 to 25 amino acids in size. In some embodiments, a peptide sequence can be based on the sequence of a protein. In some embodiments, a peptide sequence can be a random arrangement of amino acids.

The terms “polypeptide” and “peptide” are used interchangeably herein, with “peptide” typically referring to a polypeptide having a length of less than about 100 amino acids. Polypeptides may contain L-amino acids, D-amino acids, or both and may contain any of a variety of amino acid modifications or analogs known in the art. Useful modifications include, e.g., terminal acetylation, amidation, lipidation, phosphorylation, glycosylation, acylation, farnesylation, sulfation, etc.

Exemplary proteins that may be used as targeting moieties in accordance with the present invention include, but are not limited to, antibodies, receptors, cytokines, peptide hormones, proteins derived from combinatorial libraries (e.g. avimers, affibodies, etc.), and characteristic portions thereof.

In some embodiments, a targeting moiety may be an antibody and/or characteristic portion thereof. The term “antibody” refers to any immunoglobulin, whether natural or wholly or partially synthetically produced and to derivatives thereof and characteristic portions thereof. An antibody may be monoclonal or polyclonal. An antibody may be a member of any immunoglobulin class, including any of the human classes: IgG, IgM, IgA, IgD, and IgE.

As used herein, an antibody fragment (i.e. characteristic portion of an antibody) refers to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments.

An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids.

In some embodiments, antibodies may include chimeric (e.g. “humanized”) and single chain (recombinant) antibodies. In some embodiments, antibodies may have reduced effector functions and/or bispecific molecules. In some embodiments, antibodies may include fragments produced by a Fab expression library.

Single-chain Fvs (scFvs) are recombinant antibody fragments consisting of only the variable light chain (VL) and variable heavy chain (VH) covalently connected to one another by a polypeptide linker. Either VL or VH may comprise the NH2-terminal domain. The polypeptide linker may be of variable length and composition so long as the two variable domains are bridged without significant steric interference. Typically, linkers primarily comprise stretches of glycine and serine residues with some glutamic acid or lysine residues interspersed for solubility.

Diabodies are dimeric scFvs. Diabodies typically have shorter peptide linkers than most scFvs, and they often show a preference for associating as dimers.

An Fv fragment is an antibody fragment which consists of one VH and one VL domain held together by noncovalent interactions. The term “dsFv” as used herein refers to an Fv with an engineered intermolecular disulfide bond to stabilize the VH-VL pair.

A F(ab′)2 fragment is an antibody fragment essentially equivalent to that obtained from immunoglobulins by digestion with an enzyme pepsin at pH 4.0-4.5. The fragment may be recombinantly produced.

A Fab′ fragment is an antibody fragment essentially equivalent to that obtained by reduction of the disulfide bridge or bridges joining the two heavy chain pieces in the F(ab′)2 fragment. The Fab′ fragment may be recombinantly produced.

A Fab fragment is an antibody fragment essentially equivalent to that obtained by digestion of immunoglobulins with an enzyme (e.g. papain). The Fab fragment may be recombinantly produced. The heavy chain segment of the Fab fragment is the Fd piece.

In some embodiments, a targeting moiety in accordance with the present invention may comprise a carbohydrate (e.g. glycoproteins, proteoglycans, etc.). In some embodiments, a carbohydrate may be a polysaccharide comprising simple sugars (or their derivatives) connected by glycosidic bonds, as known in the art. Such sugars may include, but are not limited to, glucose, fructose, galactose, ribose, lactose, sucrose, maltose, trehalose, cellobiose, mannose, xylose, arabinose, glucoronic acid, galactoronic acid, mannuronic acid, glucosamine, galatosamine, and neuramic acid. In some embodiments, a carbohydrate may be one or more of pullulan, cellulose, microcrystalline cellulose, hydroxypropyl methylcellulose, hydroxycellulose, methylcellulose, dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In some embodiments, the carbohydrate may be aminated, carboxylated, acetylated and/or sulfated. In some embodiments, hydrophilic polysaccharides can be modified to become hydrophobic by introducing a large number of side-chain hydrophobic groups.

In some embodiments, a targeting moiety in accordance with the present invention may comprise one or more fatty acid groups or salts thereof (e.g. lipoproteins). In some embodiments, a fatty acid group may comprise digestible, long chain (e.g., C₈-C₅₀), substituted or unsubstituted hydrocarbons. In some embodiments, a fatty acid group may be a C₁₀-C₂₀ fatty acid or salt thereof. In some embodiments, a fatty acid group may be a C₁₅-C₂₀ fatty acid or salt thereof. In some embodiments, a fatty acid group may be a C₁₅-C₂₅ fatty acid or salt thereof. In some embodiments, a fatty acid group may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, thermally-responsive conjugates are not targeted to particular locales (e.g. organs, tissues, cells, subcellular locales, and/or extracellular matrix components) by any of the targeting moieties described above. In some embodiments, targeting may instead be facilitated by a property intrinsic to a thermally-responsive conjugate (e.g. geometry of the conjugate and/or conjugate assembly).

In some embodiments, an agent to be delivered may function as a targeting moiety as described herein. To give but one example, an antibody that is useful for targeting conjugates to specific tissues may also serve as a therapeutic agent. In some embodiments, the agent to be delivered may be distinct from a targeting moiety.

Nanoparticle conjugates comprising targeting moieties are described in further detail in co-pending U.S. patent application entitled “DELIVERY OF NANOPARTICLES AND/OR AGENTS TO CELLS,” filed Dec. 6, 2007 (the entire contents of which are incorporated herein by reference and are attached hereto as Appendix A).

### Single- and Multi-Conjugate Systems

The present invention provides heatable surfaces (e.g. particles) which heat in response to external stimuli (e.g. EM fields, light, etc.) and are associated with one or more agents to be delivered via thermally-responsive linkers that mediate release of the agent above a trigger temperature. In this manner, exposing the heatable surfaces to external stimuli causes them to be heated sufficiently to trigger release of the agent. In some embodiments, such systems are designed for a single release by having a uniform population of heatable surfaces and linkers. In some embodiments, by using heatable surfaces that heat at specific frequencies and linkers with varying temperatures of release, populations of particles and linkers can be triggered to emit an unlimited spectrum of complex dosages in response to imposed EM fields.

An advantage of this design is its ability to simultaneously use a wide variety of thermally-responsive linkers to release different agents at different temperatures. This is accomplished by using a multitude of thermally-responsive linkers designed to release at specific local temperatures, enabling delivery of simple or complex drug mixtures, in specific orders, over long or short periods of time. By using several different types of particles, each with preferential heating at specific frequencies, the population of thermally-responsive conjugates can be designed such that it enables release of complex drug dosages in response to imposed external stimuli (e.g. EM fields).

In some embodiments, populations of thermally-responsive conjugates are “single-component” systems. In other words, “single component” conjugates comprise heatable surfaces, thermally-responsive linkers, and/or agents to be delivered that are all identical to one another. To give but a few examples, heatable surfaces that are suitable for use in single-component conjugates may include magnetic particles (e.g. gold, silver, iron, cobalt, zinc, cadmium, nickel, gadolinium, chromium, copper, manganese, palladium, tin, etc., alloys thereof, and/or oxides thereof).

In some embodiments, conjugate systems are “two-component” or “multi-component” conjugate systems. In other words, “two-component” or “multi-component” conjugate systems (e.g. conjugate populations, pluralities of conjugates, etc.) comprise heatable surfaces, thermally-responsive linkers, and/or agents to be delivered that are not all identical to one another. In some embodiments, a two-component conjugate system comprises two populations of conjugates, wherein each population comprises different heatable surfaces. In some embodiments, a multi-component system comprises more than two populations of conjugates, wherein at least two populations comprise different heatable surfaces. In some embodiments, a two-component conjugate system comprises two populations of conjugates, wherein each population comprises different thermally-responsive linkers. In some embodiments, a multi-component system comprises more than two populations of conjugates, wherein at least two populations comprise different thermally-responsive linkers. In some embodiments, a two-component conjugate system comprises two populations of conjugates, wherein each population comprises different agents to be delivered. In some embodiments, a multi-component conjugate system comprises more than two populations of conjugates, wherein at least two populations comprise different agents to be delivered.

In some embodiments, a multi-component conjugate system comprises more than two populations of conjugates, wherein at least two populations comprise different heatable surfaces and different agents to be delivered. In some embodiments, a multi-component conjugate system comprises more than two populations of conjugates, wherein at least two populations comprise different heatable surfaces and different thermally-responsive linkers. In some embodiments, a multi-component conjugate system comprises more than two populations of conjugates, wherein at least two populations comprise different thermally-responsive linkers and different agents to be delivered. In some embodiments, a multi-component conjugate system comprises more than two populations of conjugates, wherein at least two populations comprise different heatable surfaces, different thermally-responsive linkers, and different agents to be delivered.

In some embodiments, a single thermally-responsive conjugate may comprise a particle associated with multiple different thermally-responsive linkers and multiple different agents to be delivered. In some embodiments, the multiple different thermally-responsive linkers are sensitive to different temperatures. In some embodiments, such a conjugate may be used to deliver different therapeutic agents at different points in time (i.e. a dosage schedule). To give but one example, a conjugate may comprise (i) a particle which heats upon exposure to EM fields, (ii) a first thermally-responsive linker that is disrupted at temperatures of 42° C. or greater, (iii) a first therapeutic agent associated with the first thermally-responsive linker, (iv) a second thermally-responsive linker that is disrupted at temperatures of 50° C. or greater, and (v) a second therapeutic agent associated with the second thermally-responsive linker. The particle can be subjected first to an EM field having a frequency sufficient to cause heating of the particle to a temperature of equal to or greater than 42° C., but less than 50° C., thereby causing selective release of the first therapeutic agent associated with the first thermally-responsive linker. The particle can then be subjected to an EM field having a frequency sufficient to cause heating of the particle to 50° C. or greater, thereby causing release of the second therapeutic agent associated with the second thermally-responsive linker.

In some embodiments, therapeutic effect may be enhanced by delivering a first agent, waiting for a specified period of time, and then delivering a second agent. In the previously-described example, therapeutic effect may be enhanced by delivering the chemotherapeutic agent, waiting for a specified period of time, and then delivering the siRNA. To give a specific example, a thermally-responsive conjugate similar to what is described in the previous paragraph can be used for timing the co-delivery of a chemotherapeutic agent (e.g. cisplatin) and an siRNA that is known to sensitize cells to that particular chemotherapeutic agent or to chemotherapeutic agents in general (e.g. siRNAs targeting MAPKAP kinase 2 (Reinhardt et al., 2007, Cancer Cell, 11:175; incorporated herein by reference)).

The present invention provides methods of triggering disassembly of dendrimer-like conjugate assemblies connected via heat-liable linkers. Controlled disassociation of conjugate assemblies enables timed cargo release from large aggregates for the purpose of sensing, MRI, catalysis, delivery of localized high drug dosage, gene therapy, or facilitating body clearance of particles in vivo.

In some embodiments, a population of conjugates comprises multiple individual conjugates. In some embodiments, individual conjugates within a population of conjugates are physically separated from one another. In some embodiments, individual conjugates within a population of conjugates do not interact and/or associate with one another. In some embodiments, individual conjugates within a population of conjugates are not physically separated from one another. In some embodiments, individual conjugates within a population of conjugates interact and/or associate with one another.

In some embodiments, individual conjugates within a population of conjugates interact and/or associate with one another to form assemblies of conjugates. In some embodiments, a population of conjugates comprises assemblies of individual conjugates. In some embodiments, conjugate assemblies may be characterized as having an ordered structure. In some embodiments, conjugate assemblies may be characterized as having an unordered structure.

In some embodiments, conjugate assemblies may range from about 10 nm to about 100 μm in size (e.g. as measured by diameter and/or greatest dimension). In some embodiments, conjugate assemblies may range from about 10 nm to about 50 μm, about 10 nm to about 10 μm, about 10 nm to about 5 μm, about 10 nm to about 1 μm, about 10 nm to about 500 nm, or about 10 nm to about 100 nm in size. In some embodiments, conjugate assemblies may range from about 100 nm to about 100 μm, about 100 nm to about 50 μm, about 100 nm to about 10 μm, about 100 nm to about 5 μm, about 100 nm to about 1 μm, or about 100 nm to about 500 nm in size.

In some embodiments, conjugate assemblies may be approximately 10 nm, approximately 50 nm, approximately 100 nm, approximately 250 nm, approximately 500 nm, approximately 1 μm, approximately 2 μm, approximately 3 μm, approximately 4 μm, approximately 5 μm, approximately 10 μm, approximately 25 μm, approximately 50 μm, approximately 75 μm, approximately 100 μm in size, or larger.

In some embodiments, conjugate assemblies comprise two or more individual conjugates. In some embodiments, conjugate assemblies contain approximately 2, approximately 3, approximately 4, approximately 5, approximately 10, approximately 25, approximately 50, approximately 75, approximately 100, approximately 250, approximately 500, approximately 750, approximately 1000, approximately 2500, approximately 5000, approximately 7500, approximately 10,000, or more individual conjugates.

In some embodiments, conjugate assemblies consist of or consist essentially of approximately 2, approximately 3, approximately 4, approximately 5, approximately 10, approximately 25, approximately 50, approximately 75, approximately 100, approximately 250, approximately 500, approximately 750, approximately 1000, approximately 2500, approximately 5000, approximately 7500, approximately 10,000, or more individual conjugates.

Conjugate assemblies may be formed by any method available in the art. In some embodiments, conjugate assemblies may be formed by a layer-by-layer coating process. In some embodiments, conjugate assemblies may be formed by a single-step equilibrium assembly. In some embodiments, conjugate assemblies may be formed by any aqueous and/or organic solvent process. In some embodiments, conjugate assemblies are formed by serial dilution and introduction of new conjugates to self assemble around the existing formations. In this way, assembly can be conducted in a controlled, non-aggregate manner.

Therapeutic, diagnostic, and/or prophylactic applications of single- and multi-component thermally-responsive conjugate systems and of populations of conjugates are described in further detail in the section entitled “Therapeutic Applications” (below).

### Methods of Manufacturing Thermally-Responsive Conjugates

Thermally-responsive conjugates may be manufactured using any available method. Methods of forming heatable surfaces (e.g. magnetic particles) are known in the art. For example, aqueous and organic solvent syntheses for monodisperse semiconductor, conductive, magnetic, organic, and other particles have been developed elsewhere (Pellegrino et al., 2005, Small, 1:48; Murray et al., 2000, Ann. Rev. Mat. Sci., 30:545; and Trindade et al., 2001, Chem. Mat., 13:3843; all of which are incorporated herein by reference). Alternatively or additionally, particulate formulations can be formed by methods as milling, microfabrication, nanofabrication, sacrificial layers, etc., which are known in the art (Haynes et al., 2001, J. Phys. Chem., 105:5599; incorporated herein by reference).

In general, assembly of conjugates involves at least one chemical reaction. For example, attaching the agent to be delivered to the thermally-responsive linker may take place in one reaction, and attaching the heatable surface to a thermally-responsive linker may take place in a second reaction. From this point, the conjugates are formed by self-assembly, which can be performed in a controlled manner by dictating the concentrations of the individual components (e.g. heatable surfaces, thermally-responsive linkers, agents to be delivered, etc.). For example, if particle A and particle B (each associated with a thermally-responsive linker) associate with each other and are mixed together in equal amounts, they may bind one another into large aggregates. However, if the ratio of A to B is 1:100, the particles formed may contain a single A completely surrounded by B particles. This method can be repeated for stepwise synthesis of ordered conjugate assemblies.

In some embodiments, such ratiometric assembly may be utilized to synthesize and/or form diverse structures with varying geometries and components. Conjugate assemblies may be templated to form spherical assemblies around a central sphere, worm-like structures around a central worm, and/or branched morphologies. Individual conjugates within conjugate assemblies may serve unique, complimentary, and/or contradictory purposes. In some embodiments, individual conjugates within conjugate assemblies may have properties when assembled that differ from properties of unassembled, individual conjugates (e.g. emergent pharmacokinetics, transport rates, binding affinities, electromagnetic properties, etc.).

In some embodiments, a heatable surface and a thermally-responsive linker are physically associated with one another. In some embodiments, a thermally-responsive linker and an agent to be delivered are physically associated with one another. In some embodiments, a heatable surface and an agent to be delivered are physically associated with one another. In some embodiments, a heatable surface and a targeting moiety are physically associated with one another. In some embodiments, a thermally-responsive linker and a targeting moiety are physically associated with one another. In some embodiments, an agent to be delivered and a targeting moiety are physically associated with one another. In certain specific embodiments, a heatable surface, thermally-responsive linker, and agent to be delivered are physically associated with one another. In certain specific embodiments, a heatable surface, thermally-responsive linker, agent to be delivered, and targeting moiety are physically associated with one another.

Physical association can be achieved in a variety of different ways. Physical association may be covalent or non-covalent. In some embodiments, non-covalent physical association may be characterized by association with the surface of, encapsulated within, surrounded by, and/or distributed throughout the polymeric matrix of a heatable surface.

In some embodiments, a heatable surface, thermally-responsive linker, and/or agent to be delivered may be directly conjugated to one another, e.g., by one or more covalent bonds, or may be conjugated by means of one or more linkers. In some embodiments, the linker forms one or more covalent or non-covalent bonds with the heatable surface and one or more covalent or non-covalent bonds with the thermally-responsive linker, thereby attaching them to one another. In some embodiments, a first linker forms a covalent or non-covalent bond with the heatable surface and a second linker forms a covalent or non-covalent bond with the thermally-responsive linker. The two linkers form one or more covalent or non-covalent bond(s) with each other.

In some embodiments, the linker forms one or more covalent or non-covalent bonds with the heatable surface and one or more covalent or non-covalent bonds with the agent to be delivered, thereby attaching them to one another. In some embodiments, a first linker forms a covalent or non-covalent bond with the heatable surface and a second linker forms a covalent or non-covalent bond with the agent to be delivered. The two linkers form one or more covalent or non-covalent bond(s) with each other.

In some embodiments, the linker is a cleavable linker. To give but a few examples, thermally-responsive linkers include protease thermally-responsive peptide linkers, nuclease sensitive nucleic acid linkers, lipase sensitive lipid linkers, glycosidase sensitive carbohydrate linkers, pH sensitive linkers, hypoxia sensitive linkers, photo-thermally-responsive linkers, thermally-responsive linkers, enzyme thermally-responsive linkers, ultrasound-sensitive linkers, x-ray thermally-responsive linkers, etc. In some embodiments, the linker is not a cleavable linker.

Any of a variety of methods can be used to conjugate a linker (e.g. a biomolecule such as a polypeptide, carbohydrate, or nucleic acid) to a particle (e.g. magnetic particle). General strategies include passive adsorption (e.g., via electrostatic interactions), multivalent chelation, high affinity non-covalent binding between members of a specific binding pair, covalent bond formation, etc. (Gao et al., Curr. Op. Biotechnol., 16:63).

A bifunctional cross-linking reagent can be employed. Such reagents contain two reactive groups, thereby providing a means of covalently conjugating two target groups. The reactive groups in a chemical cross-linking reagent typically belong to various classes of functional groups such as succinimidyl esters, maleimides, and pyridyldisulfides. Exemplary cross-linking agents include, e.g., carbodiimides, N-hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), N-Succinimidyl 3-[2-pyridyldithio]-propionamido (SPDP), succimidyl α-methylbutanoate, biotinamidohexanoyl-6-amino-hexanoic acid N-hydroxy-succinimide ester (SMCC), succinimidyl-[(N-maleimidopropionamido)-dodecaethyleneglycol]ester (NHS-PEO12), etc. For example, carbodiimide-mediated amide formation and active ester maleimide-mediated amine and sulfhydryl coupling are widely used approaches.

Common schemes for forming a conjugate involve the coupling of an amine group on one molecule to a thiol group on a second molecule, sometimes by a two- or three-step reaction sequence. A thiol-containing molecule may be reacted with an amine-containing molecule using a heterobifunctional cross-linking reagent, e.g., a reagent containing both a succinimidyl ester and either a maleimide, a pyridyldisulfide, or an iodoacetamide. Amine-carboxylic acid and thiol-carboxylic acid cross-linking, maleimide-sulfhydryl coupling chemistries (e.g., the maleimidobenzoyl-N-hydroxysuccinimide ester (MBS) method), etc., may be used. Polypeptides can conveniently be attached to particles via amine or thiol groups in lysine or cysteine side chains respectively, or by an N-terminal amino group. Nucleic acids such as RNAs can be synthesized with a terminal amino group. A variety of coupling reagents (e.g., succinimidyl 3-(2-pyridyldithio)propionate (SPDP) and sulfosuccinimidyl-4-(N-maleimidomethyl)cyclohexane-1-carboxylate (sulfo-SMCC) may be used to conjugate the various components of thermally-responsive conjugates. Heatable surfaces can be prepared with functional groups, e.g., amine or carboxyl groups, available at the surface to facilitate conjugation to a biomolecule.

Non-covalent specific binding interactions can be employed. For example, either a particle or a biomolecule can be functionalized with biotin with the other being functionalized with streptavidin. These two moieties specifically bind to each other non-covalently and with a high affinity, thereby conjugating the particle and the biomolecule. Other specific binding pairs could be similarly used (e.g. antibody-antigen pairs). Alternately, histidine-tagged biomolecules can be conjugated to particles conjugated with nickel-nitrolotriaceteic acid (Ni-NTA).

Any biomolecule to be attached to a heatable surface, thermally-responsive linker, and/or agent to be delivered may include a spacer. The spacer can be, for example, a short peptide chain, e.g., between 1 and 10 amino acids in length, e.g., 1, 2, 3, 4, 5, or more amino acids in length, a nucleic acid, an alkyl chain, etc.

For additional general information on conjugation methods and cross-linkers, see the journal Bioconjugate Chemistry, published by the American Chemical Society, Columbus Ohio, PO Box 3337, Columbus, Ohio, 43210; “Cross-Linking,” Pierce Chemical Technical Library, available at the Pierce web site and originally published in the 1994-1995 Pierce Catalog, and references cited therein; Wong S S, Chemistry of Protein Conjugation and Cross-linking, CRC Press Publishers, Boca Raton, 1991; and Hermanson, G. T., Bioconjugate Techniques, Academic Press, Inc., San Diego, 1996.

It is to be understood that the compositions in accordance with the invention can be made in any suitable manner, and the invention is in no way limited to compositions that can be produced using the methods described herein. Selection of an appropriate method may require attention to the properties of the particular moieties being conjugated.

If desired, various methods may be used to separate conjugates with an attached thermally-responsive linker and/or agent to be delivered from conjugates with which the thermally-responsive linker and/or agent to be delivered has not become associated, or to separate conjugates having different numbers of thermally-responsive linkers, and/or agents to be delivered attached thereto. For example, size exclusion chromatography, agarose gel electrophoresis, or filtration can be used to separate populations of conjugates having different numbers of moieties attached thereto and/or to separate conjugates from other entities. Some methods include size-exclusion or anion-exchange chromatography.

Any method may be used to determine whether aggregates of thermally-responsive conjugates have formed, including measuring extinction coefficients, atomic force microscopy (AFM), etc. An extinction coefficient, generally speaking, is a measure of a substance's turbidity and/or opacity. If EM radiation can pass through a substance very easily, the substance has a low extinction coefficient. Conversely, if EM radiation hardly penetrates a substance, but rather quickly becomes “extinct” within it, the extinction coefficient is high. For example, to determine whether aggregates of thermally-responsive conjugates have formed, EM radiation is directed toward and allowed to pass through a sample. If the sample contains primarily conjugate aggregates, EM radiation will deflect and scatter in a pattern that is different from the pattern produced by a sample containing primarily individual conjugates.

In general, AFM utilizes a high-resolution type of scanning probe microscope and attains resolution of fractions of an Angstrom. The microscope has a microscale cantilever with a sharp tip (probe) at its end that is used to scan a specimen surface. The cantilever is frequently silicon or silicon nitride with a tip radius of curvature on the order of nanometers. When the tip is brought into proximity of a sample surface, forces between the tip and the sample lead to a deflection of the cantilever according to Hooke's law. Typically, a feedback mechanism is employed to adjust the tip-to-sample distance to maintain a constant force between the tip and the sample. Samples are usually spread in a thin layer across a surface (e.g. mica), which is mounted on a piezoelectric tube that can move the sample in the z direction for maintaining a constant force, and the x and y directions for scanning the sample.

In general, forces that are measured in AFM may include mechanical contact force, Van der Waals forces, capillary forces, chemical bonding, electrostatic forces, magnetic forces, Casimir forces, solvation forces, etc. Typically, deflection is measured using a laser spot reflected from the top of the cantilever into an array of photodiodes. Alternatively or additionally, deflection can be measured using optical interferometry, capacitive sensing, or piezoresistive AFM probes.

### Applications

In some embodiments, a composition in accordance with the invention is administered to a subject for therapeutic, diagnostic, and/or prophylactic purposes. In some embodiments, the amount of thermally-responsive conjugate and/or population of thermally-responsive conjugates is sufficient to treat, alleviate symptoms of, diagnose, prevent, and/or delay the onset of a disease, condition, and/or disorder. In some embodiments, the invention encompasses “therapeutic cocktails,” including, but not limited to, approaches in which multiple thermally-responsive conjugates are administered.

The present invention provides thermally-responsive conjugates that enable delivery of an agent (e.g. therapeutic, diagnostic, and/or prophylactic agent) at a specific time. An agent to be delivered, as described herein, may be released from conjugates free in the bloodstream, from conjugates in tissues, from conjugates in cells, from conjugates within a hydrogel, from conjugates immobilized onto a surface, and/or from conjugates behind a membrane. Thermally-responsive conjugates may be used in vitro as well as in vivo.

To give but a few examples, applications include intelligent drug delivery, controllable drug implants, simplified vaccinations, more potent cancer treatments, enhanced sensing capabilities, MRI, gene therapy, monitoring enzyme catalysis of endogenous and/or delivered substrates, delivery of high drug or cargo dosages to single points, reduction of non-specific drug release, localized release of growth factors to cells, intracellular cargo delivery, and/or controlled vehicle disassembly for easing clearance of particles in vivo.

Therapeutic Applications

In some embodiments, thermally-responsive conjugates are used for delivery of a therapeutic and/or nutraceutical agent to an organ, tissue, cell, subcellular locale, and/or extracellular matrix locale. Any therapeutic and/or nutraceutical agent may be delivered using the thermally-responsive conjugates described herein, and examples of therapeutic and/or nutraceutical agents that can be delivered using thermally-responsive conjugates are described in the section entitled “Agents to be Delivered” (above). Such agents include, but are not limited to, chemotherapeutic agents, radiation-sensitizers (e.g., for radiation therapy), peptides and/or proteins that affect the cell cycle, protein toxins, vitamins, and/or any other therapeutic and/or nutraceutical agent.

The present invention provides methods for the treatment of a disease, disorder, and/or condition. In some embodiments, the treatment of a disease, disorder, and/or condition comprises administering a therapeutically effective amount of thermally-responsive conjugates to a subject in need thereof, in such amounts and for such time as is necessary to achieve the desired result. In certain embodiments, a “therapeutically effective amount” of an conjugate is that amount effective for treating, alleviating, ameliorating, relieving, delaying onset of, inhibiting progression of, reducing severity of, and/or reducing incidence of one or more symptoms or features of a disease, disorder, and/or condition.

Any disease, disorder, and/or condition may be treated using conjugates. Exemplary diseases, disorders, and/or conditions that may be treated include, but are not limited to, autoimmune disorders (e.g. diabetes, lupus, multiple sclerosis, psoriasis, rheumatoid arthritis); inflammatory disorders (e.g. arthritis, pelvic inflammatory disease); infectious diseases (e.g. viral, bacterial, and fungal infections; sepsis); neurological disorders (e.g. Alzheimer's disease, autism); cardiovascular disorders (e.g. atherosclerosis, thrombosis, clotting disorders, angiogenic disorders such as macular degeneration); proliferative disorders (e.g. cancer); respiratory disorders (e.g. chronic obstructive pulmonary disease); digestive disorders (e.g. inflammatory bowel disease, ulcers); musculoskeletal disorders (e.g. fibromyalgia, arthritis); endocrine, metabolic, and nutritional disorders (e.g. diabetes, osteoporosis); urological disorders (e.g. renal disease); liver disorders (e.g. hepatocellular carcinoma; fibrosis/cirrhosis; genetic defects; metabolic and clotting disorders, such as diabetes and obesity that are mediated through the liver; hepatitis, such as hepatitis A, B, C, and/or D; other infectious diseases, such as malaria, dengue, etc.; etc.); psychological disorders (e.g. depression, schizophrenia); skin disorders (e.g. wounds, eczema); blood and lymphatic disorders (e.g. anemia, hemophilia); etc. In some embodiments, conjugates are used to treat a cell proliferative disorder. In some embodiments, conjugates are used to treat cancer. In some embodiments, for example, a therapeutically effective amount of a thermally-responsive conjugate and/or conjugate system is that amount effective for inhibiting survival, growth, and/or spread of a tumor.

In some embodiments, the invention provides efficient and effective methods for controllable delivery of therapeutic agents utilizing thermally-responsive conjugates in accordance with the present invention. In some embodiments, the present invention provides methods for delivery of therapeutic agents which permit release of the therapeutic agent to the subject only at desired times and/or at desired locations within a subject (e.g. desired organ, tissue, cell, subcellular locale, and/or extracellular matrix component). In some embodiments, the invention provides methods for delivery of increased therapeutic dosages relative to traditional methods of drug delivery. For example, when delivering therapeutic agents with adverse side effects (e.g. chemotherapeutic and/or cytotoxic drugs) using traditional methods of drug delivery, low doses are typically administered in order to avoid adverse side effects. Using methods in accordance with the present invention, higher doses of therapeutic agents can be delivered because the therapeutic agents are released and/or delivered in a controlled manner. In some embodiments, such methods can be used to provide more potent therapies (e.g. cancer treatments, antibiotic treatments, delivery of growth factors to cells, etc.) relative to traditional treatments. In some embodiments, such methods as those described above comprise administering a therapeutically effective amount of thermally-responsive conjugates in accordance with the present invention to a subject in need thereof.

The present invention provides controllable drug implants comprising thermally-responsive conjugates in accordance with the present invention. In some embodiments, controllable drug implants may be placed at any location within a subject, including, but not limited to, subcutaneous implantation. In some embodiments, controllable drug implants may be placed within a subject for a period of days, weeks, months, or even years. In some embodiments, controllable drug implants are physically removed from the subject (e.g. surgically) after it has been implanted within a subject (e.g. after the therapeutic purpose has been served). In some embodiments, controllable drug implants may disintegrate over time, thereby not requiring physical removal of the implant. In some embodiments, controllable drug implants may be used to administer multiple doses of an agent to be delivered. For example, a controllable drug implant comprising an agent to be delivered (e.g. therapeutic, diagnostic, prophylactic, and/or other agent) may be heated to a trigger temperature (e.g. upon exposure to an EM field or light) at desired points in time in order to release doses of the therapeutic agent at the desired points in time.

The present invention provides methods for delivering agents (e.g. therapeutic, prophylactic, diagnostic, nutraceutical, etc., agents) to subcellular locales (including intracellular locales and/or cell membranes). For example, in some embodiments, conjugates may comprise targeting moieties which specifically bind to a membrane-bound target. To give another example, in some embodiments, conjugates may comprise moieties which facilitate their entry into cells. In some embodiments, conjugates may comprise targeting moieties which specifically bind to an intracellular target. Such conjugates may be useful for delivering agents which facilitate gene therapy (e.g. vectors, functional RNAs, mutagens, etc.).

In some embodiments, thermally-responsive conjugates are used for external monitoring of drug accumulation in target sites. For example, using MRI techniques, location of particles can be detected and monitored. From this information, a skilled person can infer where drug will be released.

In some embodiments, thermally-responsive conjugates are used for delivery of a prophylactic agent to an organ, tissue, cell, subcellular locale, and/or extracellular matrix locale. Any prophylactic agent may be delivered using the thermally-responsive conjugates described herein, and examples of agents that can be delivered using thermally-responsive conjugates are described in the section entitled “Agents to be Delivered” (above). Such agents include, but are not limited to, vaccines and/or any other prophylactic agent.

In some embodiments, the present invention provides simplified methods for vaccinating an individual. For example, some types of vaccines require multiple administrations of the vaccine in order for the vaccine to be effective (e.g. adults receiving the chicken pox vaccine should receive two doses scheduled 4 weeks to 8 weeks apart). Some types of vaccines require booster doses of the vaccine at some point in time after the vaccine was initially administered (e.g. a booster dose of tetanus vaccine is administered to a subject within 10 years after the previous dose of tetanus vaccine). The present invention provides simplified methods for administering multiple administrations of a vaccine. In some embodiments, thermally-responsive conjugates in accordance with the present invention comprise a vaccine component (e.g. protein/peptide antigen; live, killed, or attenuated microbe; etc.) as the agent to be delivered. Such conjugates may be subjected to an external stimulus (e.g. EM field, light) at particular points in time corresponding to the desired vaccination dosing schedule, thereby releasing the vaccine component from the conjugate according to the desired vaccination dosing schedule. In some embodiments, such methods can allow an individual to receive multiple doses of a vaccine (e.g. booster doses) without multiple visits to a physician and/or without multiple needle pricks.

In some embodiments, thermally-responsive conjugates are used for delivery of a diagnostic agent to an organ, tissue, cell, subcellular locale, and/or extracellular matrix locale. Any diagnostic agent may be delivered using the thermally-responsive conjugates described herein, and examples of diagnostic agents that can be delivered using thermally-responsive conjugates are described in the section entitled “Agents to be Delivered” (above). Such agents include, but are not limited to, radioactive moieties, radiopaque moieties, paramagnetic moieties, particles, vesicles, markers, marker enzymes (e.g., horseradish peroxidase, β-galactosidase, and/or any other enzyme suitable for marking a cell), contrast agents (e.g., for diagnostic imaging), and/or any other diagnostic agent.

The present invention provides methods for the diagnosis of a disease, disorder, and/or condition. In some embodiments, the diagnosis of a disease, disorder, and/or condition comprises administering a therapeutically effective amount of thermally-responsive conjugates to a subject in need thereof, in such amounts and for such time as is necessary to achieve the desired result. In certain embodiments of the present invention a “therapeutically effective amount” of a thermally-responsive conjugate is that amount effective for detecting, monitoring, and/or measuring the presence of one or more symptoms or features of a disease (e.g. cancer). In some embodiments, for example, a therapeutically effective amount of a thermally-responsive conjugate is that amount effective for detecting the presence and/or determining the location of a tumor. To give but one example, a thermally-responsive conjugate may be capable of being imaged directly or it may be conjugated to a ligand (e.g. DPTA) that binds a heavy metal (e.g. yttrium, indium, etc.) and/or other agent that can be imaged.

In some embodiments, the present invention provides methods of enhancing sensing capabilities. In some embodiments, conjugate assemblies are characterized by having optical, magnetic, electric, and/or other type of detectable properties that are different from the optical, magnetic, electric, and/or other type of detectable properties of the individual conjugates that make up the conjugate assemblies. For example, in some embodiments, enhanced sensing capabilities refer to the initiation of an imaging signal by disrupting a conjugate assembly by exposure to the trigger temperature. By specifically changing an imaging signal at a given time, the sensitivity of detection over background can be improved for fluorescence measurements, MRI measurements, luminescence measurements, and so forth.

To give but one example, in some embodiments, thermally-responsive conjugates can be utilized in magnetic resonance imaging (MRI) applications. MRI utilizes superparamagnetic nanoparticles to dephase populations of protons and to enhance proton relaxation. Such nanoparticles are typically administered as monomeric particles with constant imaging signatures. In some embodiments, the present invention provides methods in which, by disassembling conjugate assemblies, the specificity of MR measurements can be enhanced as conjugate assemblies are deconstructed into individual conjugates, thereby changing their MR signatures in predictable ways. Such changes can overcome a typical limitation of magnetic particles, wherein particle signatures may be mistaken for magnetic field inhomogeneities by removing contrast upon demand.

In some embodiments, thermally-responsive conjugates may be used for external monitoring of accumulation of agents to be delivered. For example, MRI can be used to monitor the distribution of thermally-responsive conjugates within a subject. Thus, it is possible to infer where a therapeutic agent may be released or may accumulate.

In some embodiments, an agent to be delivered may be a substrate for an enzyme. In such embodiments, when an agent is released from a thermally-responsive conjugate in the presence of an enzyme which acts upon the agent, catalysis may occur. In some embodiments, catalysis results in production of a detectable product (e.g. fluorescence, color change, etc.). Therefore, in some embodiments, thermally-responsive conjugates may be used to detect release of an agent from the thermally-responsive conjugate. To give but one example, a thermally-responsive conjugate may release a substrate for a tumor-associated enzyme that itself provides a detectable signal upon catalysis.

In some embodiments, such methods involve using an assembly of conjugates that, after release of the agent to be delivered, could be cleared from the body as individual conjugates or particles. This could allow for large systems of conjugates (>100 nm), holding a quantity of drug, to disassociate into conjugates or particles small enough for renal clearance (>7 nm). One problem with depending on the liver for particle removal is that many particulates are not easily cleared from it and can stay for long periods of time with unknown effect. Alternatively or additionally, particles larger than approximately 200 nm can be non-specifically filtered by the spleen. Thus, the present invention provides methods for tailoring of particle delivery schemes for renal clearance.

The present invention provides methods which offer several advantages for treatment of conditions such as cancer, where vascular permeability many increase to >200 nm, but full tumor perfusion is more limited. The present invention provides large assemblies which can be designed to escape the leaky vasculature and then be triggered to disassociate. This could allow individual conjugates to diffuse further into the tumor before delivering the agent. Such methods can result in increased drug dosage, and exposing cells to higher dosages can increase the pharmacological effect of the drug.

The present invention encompasses the recognition that aggregate disassembly can be used to sequentially disrobe layers from a conjugate assembly. Such controlled disassembly may be utilized to release a series of drugs, to expose hidden binding sites, and/or to reveal a new type of particle at the assembly surface. To give one example, such methods may be used to expose an entity (e.g. an antigenic peptide) that simulates and/or inhibits an immune response, inflammatory cascade, localized coagulation etc.

### Administration

Thermally responsive conjugates in accordance with the present invention and pharmaceutical compositions thereof may be administered using any amount and any route of administration effective for treatment. The exact amount required will vary from subject to subject, depending on the species, age, and general condition of the subject, the severity of the infection, the particular composition, its mode of administration, its mode of activity, and the like. Thermally responsive conjugates are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disorder being treated and the severity of the disorder; the activity of the specific compound employed; the specific composition employed; the age, body weight, general health, sex, and diet of the subject; the time of administration, route of administration, and rate of excretion of the specific compound employed; the duration of the treatment; drugs used in combination or coincidental with the specific compound employed; and like factors, well known in the medical arts.

Pharmaceutical compositions of the present invention may be administered by any route. In some embodiments, pharmaceutical compositions of the present invention are administered by a variety of routes, including oral, intravenous, intramuscular, intra-arterial, intramedullary, intrathecal, subcutaneous, intraventricular, transdermal, interdermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. In some embodiments, pharmaceutical compositions are administered by systemic intravenous injection, regional administration via blood and/or lymph supply, and/or direct administration to an affected site (e.g. a therapeutic implant, such as a hydrogel). In general the most appropriate route of administration will depend upon a variety of factors including the nature of the agent (e.g., its stability in the environment of the gastrointestinal tract), the condition of the subject (e.g., whether the subject is able to tolerate oral administration), etc. In specific embodiments, thermally-responsive conjugates in accordance with the present invention and/or pharmaceutical compositions thereof may be administered intravenously. In specific embodiments, thermally-responsive conjugates in accordance with the present invention and/or pharmaceutical compositions thereof may be administered intraperitoneally. In specific embodiments, thermally-responsive conjugates in accordance with the present invention and/or pharmaceutical compositions thereof may be administered intrathecally. In specific embodiments, thermally-responsive conjugates in accordance with the present invention and/or pharmaceutical compositions thereof may be administered intratumorally. In specific embodiments, thermally-responsive conjugates in accordance with the present invention and/or pharmaceutical compositions thereof may be administered intramuscularly. In specific embodiments, thermally-responsive conjugates in accordance with the present invention and/or pharmaceutical compositions thereof may be administered via vitreal administration. In specific embodiments, thermally-responsive conjugates in accordance with the present invention and/or pharmaceutical compositions thereof may be administered via a portal vein catheter. In specific embodiments, thermally-responsive conjugates in accordance with the present invention and/or pharmaceutical compositions thereof may be immobilized into a hydrogel for controlled long-term release of thermally-responsive conjugates. However, the invention encompasses the delivery of thermally-responsive conjugates and/or pharmaceutical compositions thereof by any appropriate route taking into consideration likely advances in the sciences of drug delivery.

In certain embodiments, compositions in accordance with the invention may be administered orally or parenterally at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg of subject body weight per day to obtain the desired therapeutic effect. The desired dosage may be delivered more than three times per day, three times per day, two times per day, once per day, every other day, every third day, every week, every two weeks, every three weeks, every four weeks, every two months, every six months, or every twelve months. In certain embodiments, the desired dosage may be delivered using multiple administrations (e.g., two, three, four, five, six, seven, eight, nine, ten, eleven, twelve, thirteen, fourteen, or more administrations).

It will be appreciated that thermally-responsive conjugates in accordance with the present invention and pharmaceutical compositions thereof can be employed in combination therapies. The particular combination of therapies (e.g. therapeutics or procedures) to employ in a combination regimen will take into account compatibility of the desired therapeutics and/or procedures and the desired therapeutic effect to be achieved. It will be appreciated that the therapies employed may achieve a desired effect for the same purpose (for example, conjugates useful for reducing the size of tumors may be administered concurrently with another agent useful for reducing the size of tumors), or they may achieve different effects (e.g., control of any adverse effects).

Pharmaceutical compositions in accordance with the present invention may be administered either alone or in combination with one or more other therapeutic agents. By “in combination with,” it is not intended to imply that the agents must be administered at the same time and/or formulated for delivery together, although these methods of delivery are within the scope of the invention. The compositions can be administered concurrently with, prior to, or subsequent to, one or more other desired therapeutics or medical procedures. In general, each agent will be administered at a dose and/or on a time schedule determined for that agent. Additionally, the invention encompasses the delivery of pharmaceutical compositions in combination with agents that may improve their bioavailability, reduce and/or modify their metabolism, inhibit their excretion, and/or modify their distribution within the body.

The particular combination of therapies (e.g. therapeutics and/or procedures) to employ in a combination regimen will take into account compatibility of the desired therapeutics and/or procedures and/or the desired therapeutic effect to be achieved. It will be appreciated that the therapies employed may achieve a desired effect for the same disorder (for example, a thermally-responsive conjugate may be administered concurrently with another agent used to treat the same disorder), and/or they may achieve different effects (e.g., control of any adverse effects).

In will further be appreciated that therapeutically active agents utilized in combination may be administered together in a single composition (e.g. a conjugate which comprises a particle and two different therapeutic agents associated with two different thermally-responsive linkers) or administered separately in different compositions (e.g. two pharmaceutical compositions, each comprising a conjugate comprising a different therapeutic agent).

In general, it is expected that agents utilized in combination with be utilized at levels that do not exceed the levels at which they are utilized individually. In some embodiments, the levels utilized in combination will be lower than those utilized individually.

In some embodiments, thermally-responsive conjugates which are used as therapeutic agents may be used in combination with other therapeutic agents. To give but one example, thermally-responsive conjugates used to treat tumors may be administered in combination with other agents useful in the treatment of tumors. For example, thermally-responsive conjugates may be administered in combination with traditional chemotherapy, radiation treatment, surgical removal of a tumor, administration of biologics (e.g. therapeutic antibodies), etc.

### Pharmaceutical Compositions

The present invention provides thermally-responsive conjugates comprising one or more heatable surfaces, one or more thermally-responsive linkers, and one or more agents to be delivered. In some embodiments, the present invention provides for pharmaceutical compositions comprising thermally-responsive conjugates as described herein and one or more pharmaceutically acceptable excipients. Such pharmaceutical compositions may optionally comprise one or more additional therapeutically-active substances. In accordance with some embodiments, a method of administering a pharmaceutical composition comprising thermally-responsive conjugates to a subject in need thereof is provided. In some embodiments, the compositions are administered to humans. For the purposes of the present disclosure, the phrase “active ingredient” generally refers to a thermally-responsive conjugate.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions in accordance with the invention is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations of the present invention may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, at least 96%, at least 97%, at least 98%, at least 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the formulations. Excipients such as cocoa butter and suppository waxes, coloring agents, coating agents, sweetening, flavoring, and/or perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween®20], polyoxyethylene sorbitan [Tween®60], polyoxyethylene sorbitan monooleate [Tween®80], sorbitan monopalmitate [Span®40], sorbitan monostearate [Span®60], sorbitan tristearate [Span®65], glyceryl monooleate, sorbitan monooleate [Span®80]), polyoxyethylene esters (e.g. polyoxyethylene monostearate [Myrj®45], polyoxyethylene hydrogenated castor oil, polyethoxylated castor oil, polyoxymethylene stearate, and Solutol®), sucrose fatty acid esters, polyethylene glycol fatty acid esters (e.g. Cremophor®), polyoxyethylene ethers, (e.g. polyoxyethylene lauryl ether [Brij®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic®F 68, Poloxamer®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus®, Phenonip®, methylparaben, Germall®115, Germaben®II, Neolone™, Kathon™, and/or Euxyl®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such an Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a compound in accordance with this invention may include ointments, pastes, creams, lotions, gels, powders, solutions, sprays, inhalants and/or patches. Generally, the active ingredient is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, the rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition in accordance with the invention may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and the active ingredient may constitute 0.1% to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions in accordance with the invention formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 nm to about 200 nm.

The formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of the active ingredient, and may comprise one or more of the additional ingredients described herein. A pharmaceutical composition in accordance with the invention may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 nm to about 200 nm, and may further comprise one or more of the additional ingredients described herein.

A pharmaceutical composition in accordance with the invention may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21^{st} ed., Lippincott Williams & Wilkins, 2005.

### Kits

The invention provides a variety of kits for conveniently and/or effectively carrying out methods in accordance with the present invention. Kits typically comprise one or more thermally-responsive conjugates. In some embodiments, kits comprise a collection of different thermally-responsive conjugates to be used for different purposes (e.g. diagnostics, treatment, and/or prophylaxis). Typically kits will comprise sufficient amounts of thermally-responsive conjugates to allow a user to perform multiple treatments of a subject(s) and/or to perform multiple experiments. In some embodiments, kits are supplied with or include one or more thermally-responsive conjugates that have been specified by the purchaser.

Kits may include additional components or reagents. For example, kits may comprise one or more tools and/or pieces of equipment for exposing thermally-responsive conjugates to an EM field. In some embodiments, such tools and/or pieces of equipment are known to one of ordinary skill in the art and may provide EM radiation in the kHz to GHz range (e.g. UV to infrared). In some embodiments, such tools and/or pieces of equipment are known to one of ordinary skill in the art and may provide EM radiation in the ultraviolet to infrared range. Kits may comprise one or more control thermally-responsive conjugates, e.g., positive and negative control thermally-responsive conjugates. Other components of kits may include cells, cell culture media, tissue, and/or tissue culture media.

Kits may comprise instructions for use. For example, instructions may inform the user of the proper procedure by which to prepare a pharmaceutical composition comprising thermally-responsive conjugates and/or the proper procedure for administering the pharmaceutical composition to a subject.

In some embodiments, kits include a number of unit dosages of a pharmaceutical composition comprising thermally-responsive conjugates. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical compositions, may be included to provide a kit in which a dosage is taken every day.

Kits may comprise one or more vessels or containers so that certain of the individual components or reagents may be separately housed. Kits may comprise a means for enclosing the individual containers in relatively close confinement for commercial sale, e.g., a plastic box, in which instructions, packaging materials such as styrofoam, etc., may be enclosed.

In some embodiments, kits comprise one or more thermally-responsive conjugates in accordance with the present invention. In some embodiments, such a kit is used in the treatment, diagnosis, and/or prophylaxis of a subject suffering from and/or susceptible to a disease, condition, and/or disorder (e.g. cancer). In some embodiments, such a kit comprises (i) a thermally-responsive conjugate that is useful in the treatment of cancer; (ii) a syringe, needle, applicator, etc. for administration of the to a subject; and (iii) instructions for use.

### EXEMPLIFICATION

The representative Examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following Examples contain important additional information, exemplification and guidance that can be adapted to the practice of this invention in its various embodiments and the equivalents thereof. It will be appreciated, however, that these examples do not limit the invention. Variations of the invention, now known and/or further developed, are considered to fall within the scope of the present invention as described herein and as hereinafter claimed.

### Example 1

### Complement Binding and Heat-Triggered Release

Single-stranded DNAs (ssDNAs) have been attached to gold and iron oxide particles. Complement binding was shown, and release was demonstrated with increased macroscopic temperature. These experiments were successfully used for releasing ssDNAs from aggregates in solution and from separate particles in a gel. For release from aggregates in solution, thermal triggering of aggregate disassociation was demonstrated. Particles may be released with electromagnetical excitation.

### Example 2

### Remotely Triggered Release from Magnetic Nanoparticles

### Introduction

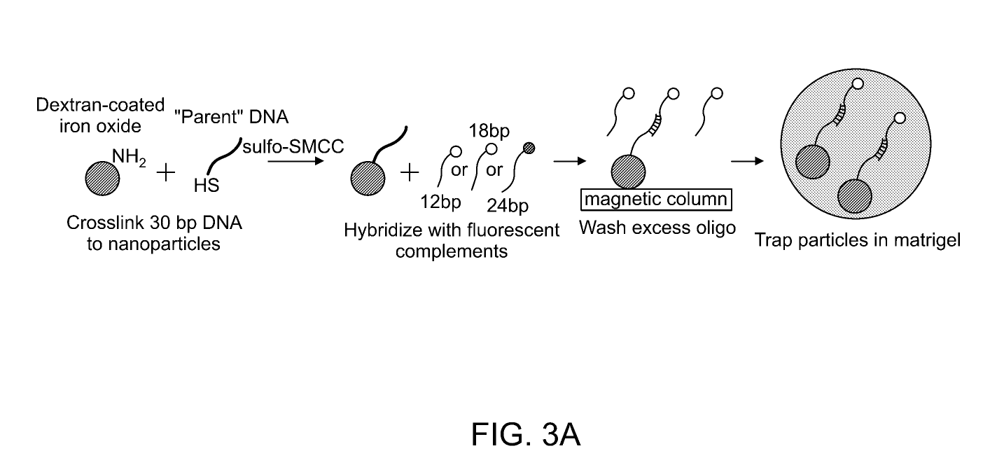

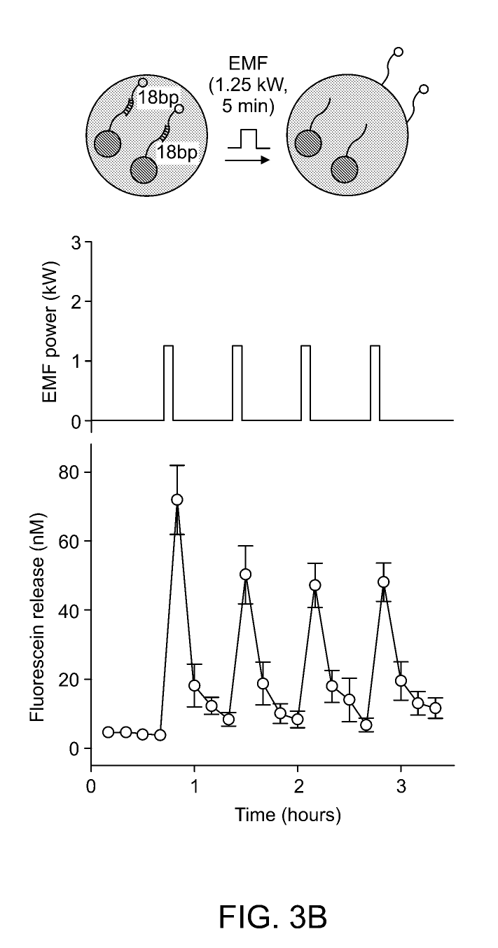

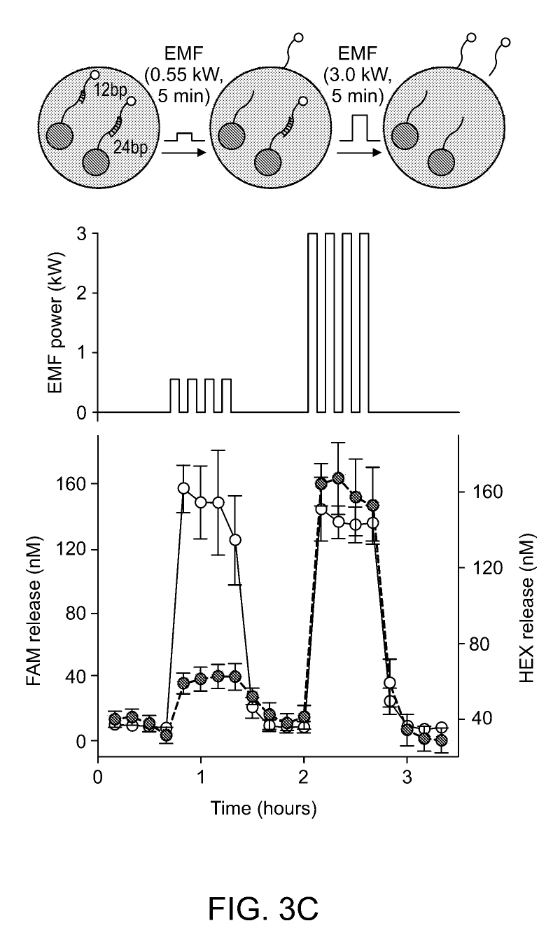

Multivalent nanoparticles have tremendous potential in the diagnosis and treatment of human disease (Ferrari, 2005, Nat. Rev. Cancer, 5:161; incorporated herein by reference). Their multivalency allows simultaneous conjugation of targeting ligands to improve nanoparticle homing, polymers (e.g. polyethylene glycol (PEG)) to improve nanoparticle pharmacokinetics, as well as therapeutic drug cargo. Drug release from a nanoparticle surface has been accomplished by bonds that are sensitive to hydrolytic degradation (Gref et al., 1994, Science, 263:1600; incorporated herein by reference) or pH (Kohler et al., 2005, Langmuir, 21:8858; incorporated herein by reference); however, complex release profiles that can be controlled from large distances (>10 cm) have not been achieved. Here, a multifunctional nanoparticle is described that is: (1) multivalent, (2) remotely-actuated, and (3) imaged non-invasively by magnetic resonance imaging (FIG. 3).

Superparamagnetic nanoparticles act as transducers to capture external electromagnetic energy at 350 kHz-400 kHz, which is not significantly absorbed by tissue, to disrupt hydrogen bonding between complementary oligonucleotides on demand. With a nucleic acid strand covalently linked to a particle (e.g. a nanoparticle), dye-labeled single stranded DNA (i.e., a model antisense therapeutic) self-assembles on the particle's surface, forming a tunable, thermally-responsive linker. The resulting multifunctional nanoparticles are used to demonstrate remote, pulsatile release of a single species and multistage release of two species in vitro, as well as noninvasive imaging and remote actuation upon implantation in vivo.

Release from surfaces or polymers triggered by an external stimulus (for example, electric current (Kwon, 1991, Nature, 354:291; and Santini et al., 1999, Nature, 397:335; both of which are incorporated herein by reference) magnetic fields (Edelman et al., 1985, J. Biomed. Mater. Res., 19:67; incorporated herein by reference); temperature (Chilkoti et al., 2002, Adv. Drug Deliv. Rev., 54:613; and Jeong et al., 2002, Adv. Drug Deliv. Rev., 54:37; both of which are incorporated herein by reference); light (Mathiowitz and Cohen, 1989, J. Membrane Sci., 40:67; incorporated herein by reference); ultrasound (Kost et al., 1989, Proc. Natl. Acad. Sci., USA, 86:7663; incorporated herein by reference); etc.) has been extensively studied (reviewed in Santini et al., 2000, Agnew Chem. Int. Edit., 39:2397; incorporated herein by reference). These strategies, however, have been principally applied to macro- and micro-scale materials and drug reservoirs. For focal diseases, such as cancer, these devices must be implanted at the tumor site (e.g. Gliadel®). Another approach is to replace these larger depots with drug-carrying nanoparticles that can be individually targeted to the tumor. Heat (Needham and Dewhirst, 2001, Adv. Drug Deliv. Rev., 53:285; incorporated herein by reference) and light-sensitive (Shum et al., 2001, Adv. Drug Deliv. Rev., 53:273; incorporated herein by reference) liposomes, for example, can be delivered systemically and their contents released in response to an external stimulus. The present invention has the added advantage of radiofrequency electromagnetic (EM) field activation, which improves penetration depth over heat or light (at 400 kHz, field penetration into 15 cm of tissue is >99% (Young et al., 1980, Electron Lett., 16:358; incorporated herein by reference). Similarly, energy absorption, and thus background heating, of water and tissue is insignificant in the 350 kHz-400 kHz frequency regime (Stauffer et al., 1984, Ieee T Biomed. Eng., 31:235; incorporated herein by reference). In contrast, when applied to magnetic materials, these fields produce heat as the magnetic dipole of the material aligns with the external field (Jordan et al., 1993, Int. J. Hyperther., 9:51; and Hergt et al., 1998, Ieee T. Magnetics, 34:3745; both of which are incorporated herein by reference).

### Materials and Methods

Particle Preparation

Synthetic 30 bp “parent” DNA (5′-Thiol-GAA GTG CGG TTA GTC GGC TTG AAT CAG CGA-3′; SEQ ID NO: 1) was conjugated to 50 nm aminated magnetite nanoparticles (dextran-coated, Micromod), using sulfo-SMCC (Sigma) as the crosslinker. As the particles were found to contain approximately 10⁴ amine groups by fluorescamine assay, a 1000-fold excess of DNA was used in a two step reaction. Particles were first reacted with crosslinker for 1 hour, filtered on a magnetic column (Miltenyi Biotec) to remove excess crosslinker, added to reduced DNA, and reacted overnight. After filtration of unconjugated parent DNA using a magnetic column, fluorescent complement DNA was added to the particles (in PBS) and allowed to hybridize overnight. The sequences used in these experiments were as follows: 24 base pair (bp) complement (5′-CGC TGA TTC AAG CCG ACT AAC CGC-3′; SEQ ID NO: 2), 18 bp complement (5′-TGA TTC AAG CCG ACT AAC-3′; SEQ ID NO: 3), and 12 bp complement (5′-TCG CTG ATT CAA-3′; SEQ ID NO: 4). Dye conjugations were performed by the DNA supplier and occurred at the 5′ end of the oligonucleotides. After hybridization, particles were filtered on a magnetic column at 4° C. to remove unbound complement.

Matrigel Plug Preparation

Phenol red free, growth factor reduced matrigel (400 μl, BD Biosciences) was added to 100 μl of particles. To obtain 1.05% total concentration of particles, 75 μl of DNA-conjugated particles (approximately 3.3 mg/ml) were added to 25 μl of similar 50 nm particles (200 mg/ml, Chemicell). Gels (total volume 500 μl) were mixed at 4° C. to prevent gelation.

In Vitro Experiments

For in vitro experiments, gels were added to polypropylene microcentrifuge tubes and incubated at 37° C. for 45 minutes to allow gelation. Gel plugs were then washed three times with 500 μl of PBS over 15 minutes. Buffer (200 μl) was added to the plugs, and sampled and replaced with fresh buffer at 10 minute intervals. When treated with EM fields during a time interval, fields were switched on for 5 minutes only, preceded by approximately 2.5 minutes and followed by approximately 2.5 minutes at room temperature. When fields were not applied during an interval, samples remained at room temperature. Supernatant samples were assayed on a plate-reader fluorometer (Molecular Devices Gemini XS) and amount of DNA quantified with standards.

In Vivo Experiments

Prior to injection of matrigel plugs into mice, approval from the Burnham Institute Animal Use Committee was obtained (AUF 05-054). In these experiments, 500 μl volumes were injected subcutaneous near the posterior mammary fat pad of six athymic nude mice and allowed to gel for 45 minutes. Prior to injection, animals were anesthetized with Avertin® (tribromoethanol) and remained under anesthesia during the remainder of the experiment. Three animals were treated with EM fields for two 5 minute doses, with 15 minutes between field applications (+EMF), while three were not treated (−EMF). For treatment, mice were placed inside a plastic tube, which was mounted inside a horizontal two-turn copper coil. One hour after EM field treatment, animals were sacrificed. Tumor phantom and surrounding tissue (fascia and skin) were removed and embedded in OTC for histology. Sections were stained with DAPI and an anti-fluorescein antibody (followed by fluorescein conjugated secondary) to amplify small signals. To quantify penetration depth, 8 images of the tissue/phantom boundary were taken for each animal (3 animals per group, 24 images total). DAPI staining was used to demarcate the boundary between the two regions. Using Metamorph software (Universal Imaging), green fluorescence on the tissue side of the boundary was quantified. For each fluorescent “object,” the area and distance from the tissue boundary was measured. An area-weighted average distance was calculated.

Radiofrequency Electromagnetic Field Applicator

A 3 kW induction heating power supply (Ameritherm Nova 3) was used with a remote heating station and custom-made coils. The coil for in vitro experiments was 2.5-turns, 12 mm ID, and resonated at 400 kHz. For the heat transfer model and mice experiments, a 2-turn, 30 mm OD coil resonating at 338 kHz was used. All coils were constructed from 4.88 mm OD copper tubing and spray-coated with insulating paint. During experiments, cooling water (10° C.-16° C.) was circulated through the coil.

MR Imaging

T1-weighted data sets of mice implanted with iron oxide particle containing gel plugs were acquired using a horizontal bore 7-Tesla imaging spectrometer (General Electric). T1-weighted acquisition was intended to achieve good anatomical detail. Data were acquired using a custom small animal imaging coil. Imaging parameters included a spin echo sequence, TR 500, TE 12, 40 mm field of view, matrix 256×256, slice thickness 0.5 mm.

### Results

In Vitro Release

A 30 bp DNA was conjugated to dextran-coated iron oxide nanoparticles and added a complement of 12 bp, 18 bp, or 24 bp linked to a model drug, a fluorophore. Excess fluorescent DNA was removed by trapping the particle on a magnetic column and washing with buffer. Particles were trapped in a hydrogel plug as an in vitro model of tumor tissue, allowing fluorescent DNA to diffuse out into the surrounding buffer only when liberated from the particles. FIG. 3B demonstrates pulsatile release of a fluorophore initiated by EM field pulses (400 kHz, 1.25 kW) of 5 minute duration every 40 minutes. The fluorescence of the surrounding buffer increased markedly in the sampling immediately after EM field application, followed by a fluorescence decrease in subsequent samplings. Because much of the fluorescent DNA rehybridized to the particles upon cooling of the plug to room temperature, subsequent EM field application allowed further release. Such a profile would be useful for metronomic dosing of a cytotoxic drug (Hanahan et al., 2000, J. Clin. Invest., 105:1045; incorporated herein by reference).

Complex Release Profiles

The use of a nucleic acid duplex as a thermally-responsive linker adds the additional feature of temperature tunability through changes in chain length and variations in G/C content. Using a variable-gain RF amplifier to control particle heating, biomolecules tethered to these oligonucleotides can be released in multiple stages. In FIG. 3C, oligonucleotides of two different lengths and corresponding fluorescent species (12 bp, FAM; 24 bp, HEX) are used to demonstrate the potential for complex release profiles. Low power EM field pulses (0.55 kW) triggered release predominantly of FAM by melting of the 12 bp complement whereas higher power (3 kW) led to simultaneous release of both species. Such a profile may be used to release multiple drugs in series, synergistic drug combinations such as a chemosensitizer and chemotherapeutic, or combination regimens such as antiangiogenic and cytotoxic compounds (Sengupta et al., 2005, Nature, 436:568; incorporated herein by reference).

Optimization of Release Conditions

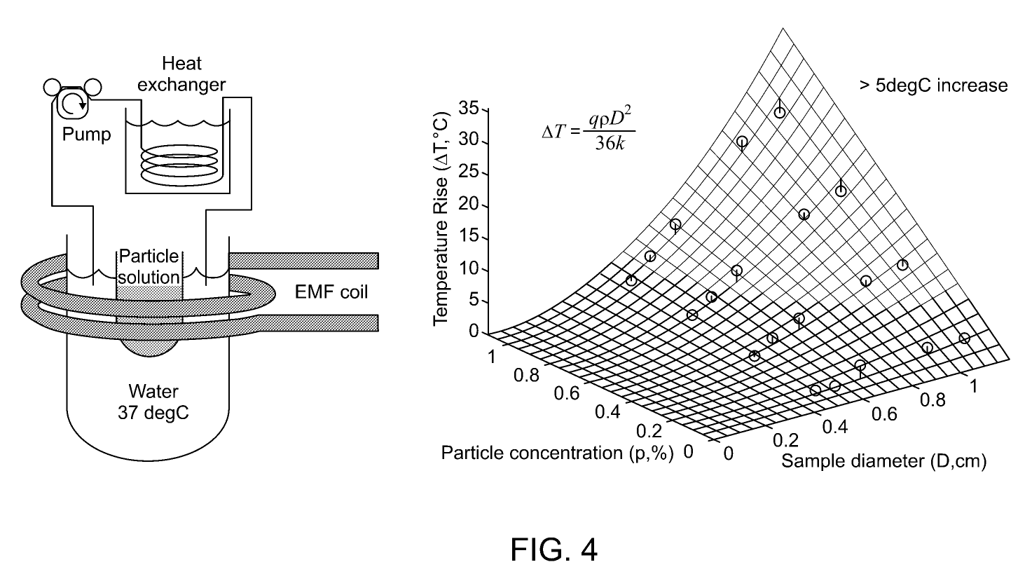

This release scheme relies on sufficient temperature rise in vivo to initiate the DNA melting. While heating sufficient to trigger release cannot be attained at the single particle level (AT approximately 10⁻⁵° C.) (Rabin, 2002, Int. J. Hyperthermia, 18:194; incorporated herein by reference), accumulation of a critical mass at a tumor site allows remote triggering through EM field application. It is therefore of interest to determine the nanoparticle concentrations required to heat various tumor diameters. Using an EMF setup and iron oxide formulation, the relationship between temperature rise, particle concentration and sample diameter was determined (FIG. 4), and these results were fitted to a conductive heat transfer model derived from Fourier's law (Rabin, 2002, Int. J. Hyperthermia, 18:194; incorporated herein by reference). Heating to 42° C. in vivo was accomplished using approximately 1.2 mg particles in a 1 cm diameter spherical tumor. For this particular experiment and these particular conditions, these data serve as an upper bound on the potential for collateral heating as the model is based on steady-state temperature rise—shorter heating intervals can be used to generate steeper temperature gradients between the tumor and surrounding tissue. For release to occur, elevated temperatures are only required for short time periods, as DNA oligonucleotides disassociate tens of microseconds after sufficient heat is applied (Ansari et al., 2001, Proc. Natl. Acad. Sci., USA, 98:7771; incorporated herein by reference). Our experiments show that reducing heating interval from 5 minutes to 30 seconds significantly reduces collateral heating. The present invention demonstrates that achieving a similar temperature rise with a shorter heating interval requires higher particle concentrations or increased EM field strength.

In Vivo Release

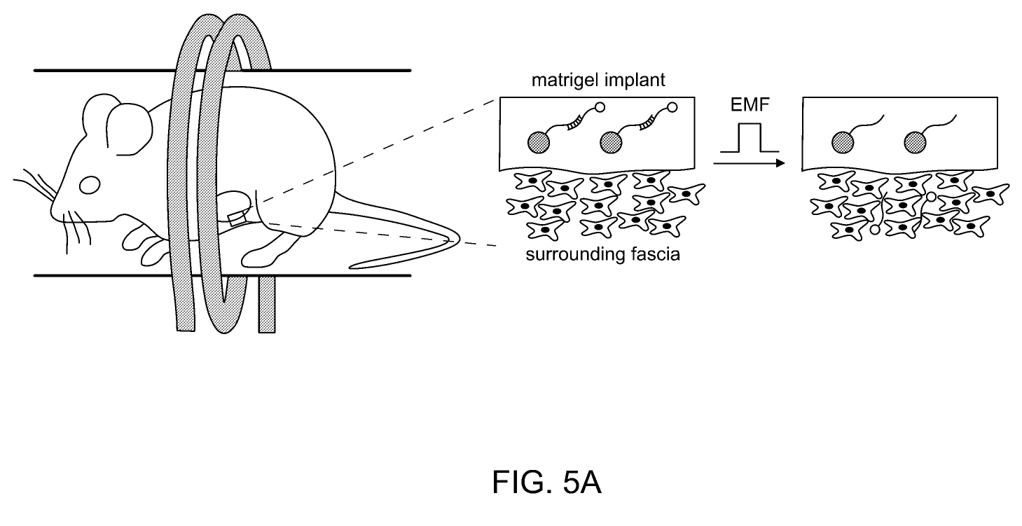

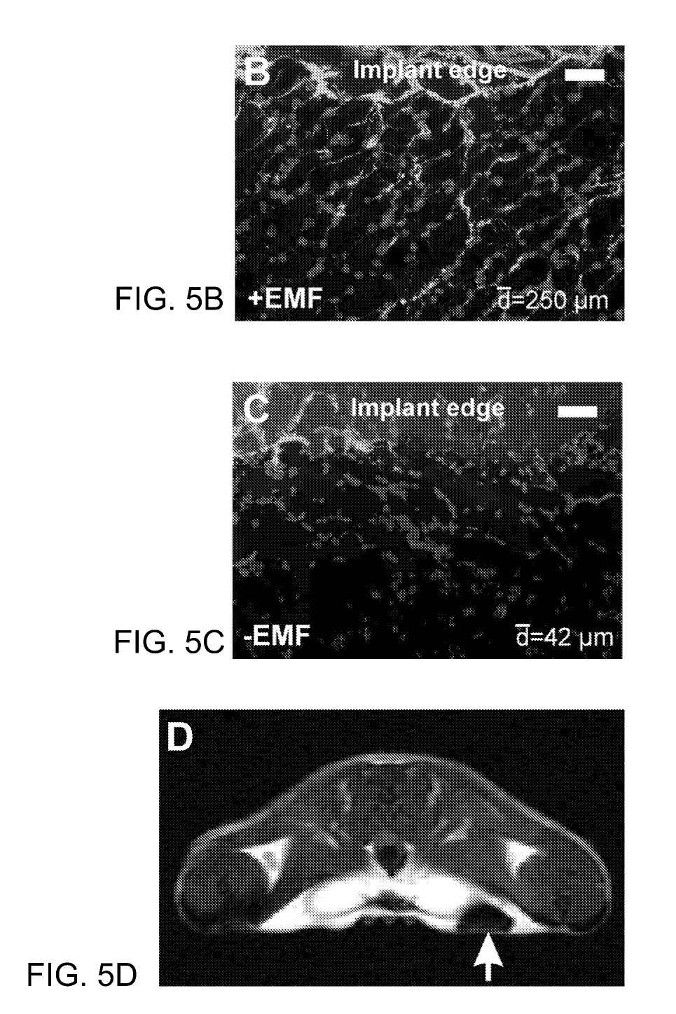

The use of the multifunctional nanoparticles in vivo was explored by implantation of a subcutaneous model tumor consisting of a matrigel plug containing nanoparticles in living mice. The release of a fluorescein-labeled 18 bp oligonucleotide was examined by EM field exposure of 3 kW for 5 minutes. After EM field treatment, tissue surrounding the plug was removed and examined for the presence of released dye. Fluorescent micrographs of histological sections in FIGS. 5B,C depict a dramatic increase in penetration depth of the model cargo into surrounding tissue due to EM field exposure. Image analysis was performed on 24 fluorescent images from each group (3 animals, 8 images each). The average distance of fluorescence signal from the tissue/phantom boundary in animals treated with EM field was approximately six-fold over unexposed controls (250±11 vs. 42±3 μm, mean±SEM). Such an increase in penetration depth could prove useful for treatment of the tumor periphery—areas often underdosed in hyperthermia generated by thermal seeds (Hilger et al., 2002, Invest. Radiol., 37:580; incorporated herein by reference). For deep-seated tumors, the use of EM field energy to break bonds remotely is an advantage over near-infrared light and other potential triggers that are more efficiently absorbed or scattered by tissue (Hirsch et al., 2003, Proc. Natl. Acad. Sci., USA, 100: 13549; incorporated herein by reference). In addition to facilitating remote actuation, the magnetic particle core allows noninvasive visualization by MRI, as depicted in FIG. 5D, suggesting the potential for simultaneous diagnosis and therapeutic delivery.

### Discussion

The present invention demonstrates the fabrication of integrated, multifunctional nanodevices which offer the potential to shift the current paradigm whereby diagnostics and therapeutics are sequential elements of patient care. For example, nanoparticles may be delivered intravascularly using homing peptides (Akerman et al., 2002, Proc. Natl. Acad. Sci., USA, 99:12617; incorporated herein by reference), used to visualize diseased tissue by MRI, and then to guide focused application of electromagnetic energy, ultimately enabling remote, physician-directed drug delivery with minimal collateral tissue exposure. The present invention encompasses the recognition that performance of these devices can be improved in the future by new materials and chemistry. Particle cores with higher magnetization results in greater heating efficiency, requiring a lower particle concentration for release. Additionally, an improved thermally-responsive tether, with a sharp temperature transition slightly above 37° C., is obtained by attaching several duplexes in parallel (Jin et al., 2003, J. Am. Chem. Soc., 125:1643; incorporated herein by reference), and non-native nucleic acids are used to mitigate the effect of nucleases. Nevertheless, the scheme outlined here demonstrates the potential to remotely trigger release of a biomolecule from the surface of a nanoparticle.

### EQUIVALENTS AND SCOPE

Those skilled in the art will recognize, or be able to ascertain using no more than routine experimentation, many equivalents to the specific embodiments of the invention, described herein. The scope of the present invention is not intended to be limited to the above Description, but rather is as set forth in the appended claims.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Thus, for example, reference to “a nanoparticle” includes a plurality of such nanoparticle, and reference to “the cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any heatable surface, any thermally-responsive linker, any trigger temperature, any agent to be delivered, any pharmaceutical composition, any method of administration, any method of use, etc.) can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

1. A conjugate, comprising:
(i) a heatable surface, wherein the heatable surface heats in response to an external stimulus,
(ii) at least one agent to be delivered; and
(iii) at least one thermally-responsive linker, wherein a first end of the thermally-responsive linker is associated with the heatable surface, wherein a second end of the thermally-responsive linker is associated with the agent to be delivered, and wherein the thermally-responsive linker is disrupted at a trigger temperature;
wherein the agent to be delivered is released from the conjugate when the thermally-responsive linker is heated to the trigger temperature or to temperatures higher than the trigger temperature.

2-4. (canceled)

5. The conjugate of claim 1, wherein the heatable surface comprises a nanoparticle.

6. (canceled)

7. The conjugate of claim 1, wherein the external stimulus is an electromagnetic (EM) field.

8. (canceled)

9. The conjugate of claim 1, wherein the thermally-responsive linker is selected from the group consisting of nucleic acids, peptides, proteins, lipids, carbohydrates, and polymers.

10. (canceled)

11. The conjugate of claim 10, wherein the thermally-responsive linker is a nucleic acid, wherein the nucleic acid comprises a duplex region, and wherein the duplex region comprises two nucleic acid strands that are associated with one another via Watson-Crick base pairing.

12-13. (canceled)

14. The conjugate of claim 10, wherein the thermally-responsive linker is a single-stranded nucleic acid.

15-57. (canceled)

58. The conjugate of claim 1, wherein the agent to be delivered is a therapeutic agent, diagnostic agent, prophylactic agent, or nutraceutical agent.

59-79. (canceled)

80. A pharmaceutical composition comprising the conjugate of claim 1 and a pharmaceutically acceptable excipient.

81. A plurality of the conjugates of claim 1, wherein all of the conjugates of the plurality of conjugates are identical to one another.

82. A plurality of the conjugates of claim 1, wherein the plurality of conjugates comprises one or more populations of non-identical conjugates.

83-89. (canceled)

90. A pharmaceutical composition comprising the plurality of conjugates of claim 81 or 82 and a pharmaceutically acceptable excipient.

91. A method of treating a disease, condition, or disorder comprising administering the conjugate of claim 1 to a subject.

92. A method of treating a disease, condition, or disorder comprising administering the plurality of conjugates of claim 81 to a subject.

93. A method of treating a disease, condition, or disorder comprising administering the plurality of conjugates of claim 82 to a subject.

94. (canceled)

95. A method, comprising steps of:
providing a subject; and
administering the pharmaceutical composition of claim 80 or 90 to a subject.

96-99. (canceled)

100. A method, comprising:
providing at least one heatable surface, at least one thermally-responsive linker, and at least one therapeutic agent to be delivered; and
allowing the at least one heatable surface, at least one thermally-responsive linker, and at least one therapeutic agent to be delivered to self-assemble such that thermally-responsive conjugates are formed.

101. A method, comprising:
providing at least one heatable surface, at least one thermally-responsive linker, and at least one therapeutic agent to be delivered; and
covalently associating the at least one heatable surface, at least one thermally-responsive linker, and at least one therapeutic agent to be delivered to one another such that thermally-responsive conjugates are formed.

102. (canceled)

103. In a method of delivering an agent, the improvement that comprises:
delivering the agent in the context of a conjugate of the agent and a heatable surface that heats in response to an external stimulus,
wherein the agent and core are associated with one another in the conjugate by means of a thermally-responsive linker that breaks at or above a trigger temperature such that, when the conjugate is exposed to the stimulus, the core heats to a temperature at or above the trigger temperature and the linker breaks so that the agent is released.


SEQUENCE LISTING (4 sequences)

SEQ ID NO: 1 - DNA, 30 bp, Artificial sequence
  parent DNA
gaagtgcggttagtcggcttgaatcagcga

SEQ ID NO: 2 - DNA, 24 bp, Artificial sequence
  24 base pair complement
cgctgattcaagccgactaaccgc

SEQ ID NO: 3 - DNA, 18 bp, Artificial sequence
  18 base pair complement
tgattcaagccgactaac

SEQ ID NO: 4 - DNA, 12 bp, Artificial sequence
  12 base pair complement
tcgctgattcaa